SEQUENCE LISTING (24 sequences)

SEQ ID NO: 1 - DNA, 24 bp, Artificial Sequence
  Synthetic APC fwd-1
cacctccaccagagcactacgttc

SEQ ID NO: 2 - DNA, 24 bp, Artificial Sequence
  Synthetic APC rev- 1
aaacgaacgtagtgctctggtgga

SEQ ID NO: 3 - DNA, 24 bp, Artificial Sequence
  Synthetic APC fwd-2
caccgtctcctgaacgtagtgctc

SEQ ID NO: 4 - DNA, 24 bp, Artificial Sequence
  Synthetic APC rev- 2
aaacgagcactacgttcaggagac

SEQ ID NO: 5 - DNA, 24 bp, Artificial Sequence
  Synthetic APC fwd-3
caccctaatgtgctgtggcactgt

SEQ ID NO: 6 - DNA, 24 bp, Artificial Sequence
  Synthetic APC rev- 3
aaacacagtgccacagcacattag

SEQ ID NO: 7 - DNA, 24 bp, Artificial Sequence
  Synthetic APC fwd-4
caccgggctcactctgaacggagc

SEQ ID NO: 8 - DNA, 24 bp, Artificial Sequence
  Synthetic APC rev- 4
aaacgctccgttcagagtgagccc

SEQ ID NO: 9 - DNA, 24 bp, Artificial Sequence
  Synthetic TP53 fwd- 1
cacctcatccagccagttcgtgac

SEQ ID NO: 10 - DNA, 24 bp, Artificial Sequence
  Synthetic TP53 rev- 1
aaacgtcacgaactggctggatga

SEQ ID NO: 11 - DNA, 24 bp, Artificial Sequence
  Synthetic TP53 fwd- 2
caccgcagctatgatttccgtcta

SEQ ID NO: 12 - DNA, 24 bp, Artificial Sequence
  Synthetic TP53 rev- 2
aaactagacggaaatcatagctgc

SEQ ID NO: 13 - DNA, 24 bp, Artificial Sequence
  Synthetic TP53 fwd- 3
caccgtccccagtcacgaactggc

SEQ ID NO: 14 - DNA, 24 bp, Artificial Sequence
  Synthetic TP53 rev- 3
aaacgccagttcgtgactggggac

SEQ ID NO: 15 - DNA, 24 bp, Artificial Sequence
  Synthetic TP53 fwd- 4
cacctgctgtccccagtcacgaac

SEQ ID NO: 16 - DNA, 24 bp, Artificial Sequence
  Synthetic TP53 rev- 4
aaacgttcgtgactggggacagca

SEQ ID NO: 17 - DNA, 24 bp, Artificial Sequence
  Synthetic SMAD fwd-1
cacctgtcctattgttactgtcga

SEQ ID NO: 18 - DNA, 24 bp, Artificial Sequence
  Synthetic SMAD rev-1
aaactcgacagtaacaataggaca

SEQ ID NO: 19 - DNA, 24 bp, Artificial Sequence
  Synthetic SMAD fwd-2
cacctcaagtaggagagacgttta

SEQ ID NO: 20 - DNA, 24 bp, Artificial Sequence
  Synthetic SMAD rev-2
aaactaaacgtctctcctacttga

SEQ ID NO: 21 - DNA, 24 bp, Artificial Sequence
  Synthetic SMAD fwd-3
caccgcaatggaacaccaatactc

SEQ ID NO: 22 - DNA, 24 bp, Artificial Sequence
  Synthetic SMAD rev-3
aaacgagtattggtgttccattgc

SEQ ID NO: 23 - DNA, 24 bp, Artificial Sequence
  Synthetic SMAD fwd-4
cacctatccatcgacagtaacaat

SEQ ID NO: 24 - DNA, 24 bp, Artificial Sequence
  Synthetic SMAD rev-4
aaacattgttactgtcgatggata

(19) United States
(12) Patent Application Publication
(10) Pub. No.: US 2023/0101335 A1
(43) Pub. Date: Mar. 30, 2023

(54) EX VIVO TISSUE EXPLANT AND GRAFT PLATFORM AND USES THEREOF

(71) Applicants: Massachusetts Institute of Technology, Cambridge, MA (US); The Brigham and Women's Hospital, Inc., Boston, MA (US)

(72) Inventors: Carlo Giovanni Traverso, Newton, MA (US); Yunhua Shi, Belmont, MA (US); Vance Soares, Boston, MA (US); Daniel Reker, Durham, NC (US); Robert S. Langer, Newton, MA (US)

(21) Appl. No.: 17/738,843

(22) Filed: May 6, 2022

Related U.S. Application Data
(60) Provisional application No. 63/184,967, filed on May 6, 2021.

(51) Int. Cl. G01N 33/50; C12N 5/09; C12N 5/071

(52) U.S. Cl. CPC G01N 33/5011; C12N 5/0693; C12N 5/0697; G01N 33/5088

(57) ABSTRACT

An ex vivo tissue composition comprising an isolated tissue explant and tissue graft is provided. Methods of making and using the tissue composition are also provided.

### CROSS-REFERENCE TO RELATED APPLICATIONS

This application claims the benefit of U.S. Provisional Patent Application Ser. No. 63/184,967, filed May 6, 2021, the entire contents of which are incorporated herein by reference.

### STATEMENT AS TO FEDERALLY SPONSORED RESEARCH

This invention was made with Government support under Grant No. R01 EB000244 awarded by the National Institutes of Health (NIH). The Government has certain rights in the invention.

### REFERENCE TO SEQUENCE LISTING

The instant application contains a Sequence Listing which has been submitted electronically in ASCII format and is hereby incorporated by reference in its entirety. Said ASCII copy, created on May 5, 2022, is named Sequence Listing_127299_02302.txt and is 4,966 bytes in size.

### BACKGROUND

Cancer is one of the most prevalent diseases in the world. Unfortunately, tumors develop resistance to existing therapeutics through changes in drug metabolism and transport. This evasion of therapy leads to multi-drug resistance and contributes to the ongoing prevalence of cancer. Current tumor models for researching drug resistance have profound differences in genetic and epigenetic variations within human tumors. Although various models have emerged for studying cancer, there is an unmet need for an in vitro experimental platform that can accurately capture similar tumor physiology to that of in vitro models. Experimental models that can accurately capture the effect of cytotoxicity and drug transport on therapeutic efficacy would be an invaluable tool for drug development and cancer biology.

### SUMMARY OF THE DISCLOSURE

In some aspects, the disclosure provides an ex vivo tissue composition comprising (i) a tissue explant from a mammalian source tissue in planar contact with a substrate; and (ii) a population of cells or tissue within the tissue explant, wherein the population of cells or tissue provides one or more biological functions of normal or diseased cells or tissue, or one or more markers of said biological functions. In some aspects, the source tissue is selected from gastrointestinal tissue, liver tissue, heart tissue, skin tissue, pancreas tissue, and kidney tissue.

In other aspects, the disclosure provides an ex vivo tissue composition comprising (i) a tissue explant in planar contact with a substrate thereby providing a luminal and a basolateral surface, wherein the tissue explant comprises epithelium from a mammalian gastrointestinal tract comprising an architecture, wherein said tissue explant comprises said architecture; and (ii) a population of cells or tissue within the tissue explant. In some aspects, the tissue explant is derived from ileum, jejunum, stomach, duodenum, esophagus, buccal, lingual, or colon of the gastrointestinal tract.

In any of the foregoing or related aspects, the population of cells or tissue is a xenograft. In some aspects, the population of cells or tissue is an allograft or an autograft. In some aspects, the population of cells or tissue is an allograft. In some aspects, the population of cells or tissue is an autograft.

In any of the foregoing or related aspects, the population of cells or tissue is derived from a primary tissue. In some aspects, the population of cells or tissue is a biopsy from a subject. In some aspects, the subject is a human.

In any of the foregoing or related aspects, the population of cells or tissue is an organoid. In some aspects, the organoid comprises cells of an immortalized cell line. In some aspects, the organoid comprises primary cells.

In any of the foregoing or related aspects, the population of cells or tissue comprises stem cells. In some aspects, the population of cells or tissue is from normal tissue. In other aspects, the population of cells or tissue is from diseased tissue. In some aspects, diseased tissue is a cancerous tissue or a tissue comprising a population of cells comprising at least one genetic mutation. In some aspects, the at least one genetic mutation is endogenous to the tissue. In other aspects, the at least one genetic mutation is introduced into the population of cells. In some aspects, the genetic mutation is in at least one of the APC, p53, or SMAD4 genes. In some aspects, the at least one genetic mutation is a knock-out or knock-down. In some aspects, the genetic mutations comprise an APC knock-out, a p53 knock-out, and a SMAD4 knock-out. In some aspects, the at least one genetic mutation is introduced via a CRISPR/Cas gene editing system. In some aspects, the cancerous tissue is derived from a tumor. In some aspects, the cancerous tissue or tumor is from colorectal cancer.

In any of the foregoing or related aspects, about 100, about 250, about 500, about 1000, about 1500, about 2000, about 2500, or about 3000 organoids are placed within the tissue explant. In some aspects, about 2000 organoids are place within the tissue explant. In some aspects, the organoids form tumors after placement within the tissue explant.

In any of the foregoing or related aspects, the substrate comprises a plurality of microwells, and wherein the population of cells or tissue is placed within the tissue explant in a location that corresponds to a microwell.

In any of the foregoing or related aspects, the population of cells or tissue is derived from tissue of the gastrointestinal tract, liver, pancreas, kidney, spleen, lung, skin or heart. In some aspects, the population of cells or tissue is derived from ileum, jejunum, stomach, duodenum, esophagus, buccal, lingual, or colon of the gastrointestinal tract.

In any of the foregoing or related aspects, the architecture of the mammalian gastrointestinal tract comprises epithelial cells having a polarity. In some aspects, the architecture comprises small intestine epithelium, circular muscular layer, mucosa layer, submucosa layer, and/or intestinal villi.

In any of the foregoing or related aspects, the tissue explant comprises a fully intact extracellular matrix. In some aspects, the fully intact extracellular matrix comprises lamina propria, lamina muscularis, or lamina propria and lamina muscularis. In some aspects, the tissue explant comprises one or more of intestinal enterocytes, tight junctions, mucin secreting goblet cells, intestinal stem cells, intestinal endocrine cells, microfold cells, mucosubstances, intact crypts, or neural cells. In some aspects, the tissue explant comprises at least one drug transporter. In some aspects, the tissue explant comprises at least one metabolizing enzyme. In some aspects, the tissue explant mimics in vivo architecture of the gastrointestinal tract from which it was derived.

In any of the foregoing or related aspects, the tissue explant comprises an intestinal mucosal layer and a submucosal layer. In some aspects, the population of cells or tissue is between the mucosa layer and the submucosa layer. In some aspects, the tissue explant comprises more than one layer and the population of cells or tissue is within a layer or between intestinal layers. In some aspects, the tissue explant comprises more than one intestinal layer and the population of cells or tissue is within an intestinal layer or between intestinal layers. In some aspects, the population of cells or tissue is injected into the tissue explant.

In any of the foregoing or related aspects, the composition is maintained in culture for 24 hours, 2 days, 3 days, 1 week, 2 weeks, 3 weeks, 4 weeks, or more. In some aspects, the composition does not require an exogenous growth factor to be maintained in culture. In some aspects, the mammal is between 3 week and 12 weeks of age. In some aspects, blood content of the tissue explant has been minimized.

In any of the foregoing or related aspects, the substrate comprises a plurality of microwells. In some aspects, the tissue explant is in planar contact with more than one microwell of the substrate. In some aspects, the substrate comprises 6, 12, 24, 48, 996, 384, or 1536 microwells. In some aspects, each microwell is completely covered by the tissue explant.

In any of the foregoing or related aspects, more than one population of cells or tissue is placed within the tissue explant. In some aspects, the number and location of population of cells or tissue placed within the tissue explant corresponds to the number and location of microwells in a substrate comprising a plurality of microwells. In some aspects, each microwell is completely covered by the tissue explant.

In any of the foregoing or related aspects, the substrate does not comprise exogenous extracellular matrix.

In any of the foregoing or related aspects, the mammalian source is a non-human mammal. In some aspects, the mammalian source is a large mammal.

In any of the foregoing or related aspects, the population of cells or tissue is three-dimensional. In some aspects, the population of cells are in contact with a biocompatible scaffold to provide a three-dimensional structure.

In any of the foregoing or related aspects, the one or more biological functions of normal cells or tissue is cell proliferation. In some aspects, the one or more biological functions of diseased cells or tissue is cell hyperproliferation.

In other aspects, the disclosure provides an ex vivo tissue composition comprising: (i) a tissue explant in planar contact with a substrate thereby providing a luminal and a basolateral surface, wherein the tissue explant comprises intestinal epithelium from a source tissue, wherein said source tissue comprises intestinal epithelium from a large mammalian gastrointestinal tract, wherein said source tissue comprises an architecture comprising epithelial cells having a polarity, wherein the tissue explant comprises said architecture; and (ii) a tumorigenic intestinal organoid within the tissue explant.

In any of the foregoing or related aspects, the tumorigenic intestinal organoid is derived from healthy intestinal tissue and at least one population of cells in the healthy tissue is gene-edited to induce formation of the tumorigenic organoid. In some aspects, the at least one population of cells is gene-edited to knock-down or knock-out expression of SMAD4, APC, p53, and any combination thereof. In some aspects, the healthy tissue is gene-edited via a CRISR/Cas9 system. In some aspects, the tumorigenic intestinal organoid is derived from cancerous intestinal tissue. In some aspects, the tumorigenic intestinal organoid is derived from human intestinal tissue. In some aspects, the tumorigenic intestinal organoid is derived from non-human intestinal tissue. In some aspects, the non-human intestinal tissue is from a large non-human mammal. In some aspects, the non-human intestinal tissue is from a pig.

In any of the foregoing or related aspects, the tissue explant and tumorigenic intestinal organoid are derived from tissues of the same species. In other aspects, the tissue explant and tumorigenic intestinal organoid are derived from tissues of different species.

In any of the foregoing or related aspects, about 100, about 250, about 500, about 1000, about 1500, about 2000, about 2500, or about 3000 tumorigenic intestinal organoids are placed within the tissue explant. In some aspects, about 2000 tumorigenic intestinal organoids are place within the tissue explant.

In any of the foregoing or related aspects, the tumorigenic intestinal organoids form tumors after placement within the tissue explant.

In any of the foregoing or related aspects, the substrate comprises a plurality of microwells, and wherein the tumorigenic intestinal organoid is placed within the tissue explant in a location that corresponds to a microwell.

In some aspects, the architecture comprises small intestine epithelium, circular muscular layer, and intestinal villi. In some aspects, the tissue explant comprises a fully intact extracellular matrix. In some aspects, the fully intact extracellular matrix comprises lamina propria, lamina muscularis, or lamina propria and lamina muscularis. In some aspects, the tissue explant comprises one or more of intestinal enterocytes, tight junctions, mucin secreting goblet cells, intestinal stem cells, intestinal endocrine cells, microfold cells, mucosubstances, intact crypts, or neural cells. In some aspects, the tissue explant comprises at least one drug transporter. In some aspects, the tissue explant comprises at least one metabolizing enzyme. In some aspects, the tissue explant mimics in vivo architecture of the gastrointestinal tract from which it was derived.

In any of the foregoing or related aspects, the tissue explant comprises an intestinal mucosal layer and the tumorigenic organoid is placed under the intestinal mucosal layer.

In any of the foregoing or related aspects, the composition is maintained in culture for 24 hours, 2 days, 3 days, 1 week, 2 weeks, 3 weeks, 4 weeks, or more. In some aspects, the composition does not require an exogenous growth factor to be maintained in culture. In some aspects, the large mammal is between 3 week and 12 weeks of age. In some aspects, blood content of the tissue explant has been minimized.

In any of the foregoing or related aspects, the tissue explant comprises more than one intestinal layer and the tumorigenic intestinal organoid is placed within an intestinal layer or between intestinal layers. In some aspects, the tumorigenic intestinal organoid is placed within the tissue explant via injection.

In any of the foregoing or related aspects, the substrate comprises a plurality of microwells. In some aspects, the tissue explant is in planar contact with more than one microwell of the substrate. In some aspects, the substrate comprises 6, 12, 24, 48, 996, 384, or 1536 microwells.

In any of the foregoing or related aspects, more than one tumorigenic intestinal organoid is within the tissue explant. In some aspects, the number and location of tumorigenic intestinal organoids placed within the tissue explant corresponds to the number and location of microwells in a substrate comprising a plurality of microwells. In some aspects, each microwell is completely covered by the tissue explant.

In any of the foregoing or related aspects, the substrate comprises a first plate comprising the plurality of microwells and a second plate, wherein the tissue explant is between the first and second plates. In some aspects, the first and second plates apply pressure to the tissue explant to minimize well-to-well leakage. In some aspects, the second plate comprises a plurality of microwells. In some aspects, the plurality of microwells of the first and second plates are through holes. In some aspects, the plurality of microwells of the first plate are through holes, and wherein the plurality of microwells of the second plate are receiving chambers. In some aspects, the first and second plates are mounted to a third plate comprising a plurality of receiving chambers.

In some aspects, the disclosure provides a method for determining the cytotoxic effect of a candidate drug on cancer cells, comprising:
(i) providing a tissue composition described herein;
(ii) contacting the tissue composition with the candidate drug; and
(iii) measuring cancer cell death within the population of cells, tissue or tumorigenic organoid after treatment with the candidate drug for a period of time, thereby determining the cytotoxic effect of the candidate drug on cancer cells.

In other aspects, the disclosure provides a method for determining the cytotoxic effect of a candidate drug on cancer cells, comprising:
(i) providing a tissue composition described herein,
(ii) conducting at least one first assay on the tissue composition by contacting the tissue explant with the candidate drug;
(iii) conducting at least one second assay on the tissue composition; and
(iv) measuring cancer cell death within the population of cells, tissue or tumorigenic organoid by comparing the first and the second assay, wherein the first and second assay are the same assay, thereby determining the effect of the compound.

In yet other aspects, the disclosure provides a system for use in a high-throughput colorectal cancer cytotoxicity screening assay, wherein the system comprises:
(i) a substrate comprising a plurality of microwells;
(ii) a tissue explant from a source tissue comprising epithelium from a large mammalian gastrointestinal tract, wherein the gastrointestinal tract epithelium comprises epithelial cells having a polarity in the tissue explant, and
(iii) a tumorigenic colon organoid injected within the tissue explant;
thereby allowing measurement of cytotoxicity toward the tumorigenic organoid through the tissue explant.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1A is a schematic demonstrating the impact of the microenvironment on the efficacy of anti-tumor drugs.

FIG. 1B is a schematic showing a workflow chart for producing an ex vivo colorectal cancer (CRC) platform. Normal intestinal organoid is isolated from healthy pig followed with sequential knock-out tumor suppressor genes to develop CRC organoids. The CRC organoids are engrafted under the mucosal layer of a freshly isolated intestine, and the system undergoes tissue culture. Drugs of interest can be added on the sample well of the platform followed by analysis to obtain the cytotoxicity result for each drug.

FIG. 2A provides images showing nutrition-selection for organoids with targeted gene knock-out (k/o). For APC⁻, the successful k/o organoids survived in medium without Wnt and R-spondin; TP53⁻ organoids were selected by the addition of Nutlin-3; and SMAD4⁻ k/o organoids survived through the addition of TGF-β.

FIG. 2B is a Western-blot demonstrating the diminished expression of target genes and their down-stream targets in intestinal organoids after CRISPR/Cas knock-out. Intestinal organoids with the following knock-outs were compared to control: APC−; APC−+P53−; APC−+P53−+SMAD4−.

FIG. 3A is a schematic showing generation of CRC organoids by CRISPR/Cas knock-out which were then administered to mice.

FIG. 3B provides images of healthy organoids, CRC organoids, and CRC organoids placed into mice that developed into (top). Hematoxylin and eosin (H&E) staining and β-catenin immunohistochemistry were applied for the confirmation of tumor growth (bottom).

FIG. 3C provides images of freshly dissected swine intestinal tissue with ex vivo engraftment of CRC organoids and then embedded between two magnetic plates. Tumor growth and tissue integrity was monitored at different time points as indicated by H&E, β-catenin and KRT20 staining.

FIGS. 4A-4F provide graphs showing dose-response of CRC organoids placed under the mucosal layer of swine intestinal tissue and placed between two magnetic plates have a plurality of microwells. Drugs at various concentrations were incubated in each well and viability of the CRC was measured via flow cytometry. Results for 5-FU (FIG. 4A), Irinotecan (FIG. 4B), Oxalipatin (FIG. 4C), Regorafenib (FIG. 4D), Capecitabine (FIG. 4E) and Leucovorin (FIG. 4F) are shown. For each drug, the percentage of cancer cell survival rate was normalized by the amount of tumor cell from the control wells. IC₅₀ value is calculated from the symmetry sigmoidal fitting of the plot of drug concentration against tumor cell survival rate. Each data point is the average among four individual experiments. Error bar stands for the standard deviation.

FIG. 5 provides graphs showing cytotoxicity measure of Doxirubicin (top), Oxaliplatin (middle) and everolimus (bottom) in the presence and absence of Pgp inhibitor CBF and lapatinib of four colorectal cancer cell lines (HT29, HCT15, Colo320DM and Caco2).

FIG. 6 provides graphs showing cytotoxicity measure of Doxirubicin (top), Oxaliplatin (middle) and everolimus (bottom) in the presence and absence of Pgp inhibitor CBF or lapatinib of CRISPR-engineered CRC organoids.

FIG. 7 provides graphs showing cytotoxicity measure of Doxirubicin (top), Oxaliplatin (middle) and everolimus (bottom) in the presence and absence of Pgp inhibitor CBF or lapatinib of the ex vivo CRC platform organoids.

FIG. 8A is a schematic showing a developing machine learning prediction algorithm to predict new P-gp modulators from databases of approved drugs, food additives and nutrients.

FIG. 8B is a graph showing the cytotoxicity measurement of selected candidates identified from the algorithm shown in FIG. 8A in combination with irinotectan in the ex vivo CRC platform.

FIG. 8C is a graph showing IC₅₀ values calculated based on FIG. 8B. Significance between each combinational treatment and irenotecan alone is calculated based on one-way ANOVA (*, p<0.05).

### DETAILED DESCRIPTION

The present disclosure is based, at least in part, on the discovery that tissue grafts (i.e., a population of cells or tissue providing one or more biological functions of normal or diseased cells or tissue, or one or more markers of said biological functions) placed within a tissue explant function as a physiologically relevant disease model for drug screening. As demonstrated herein, placement of a tumorigenic organoid in an intestinal tissue explant creates a platform useful for screening cancer therapeutics and drug penetration through the intestine to the tumor microenvironment. Tumorigenic organoids were generated from healthy intestinal tissue using gene-editing techniques (i.e. CRISPR/Cas9). Successful placement within an intestinal tissue explant enabled screening of various compounds for their anti-tumor efficacy and/or impact on drug resistance. Without wishing to be bound by theory, placement of a population of cells or tissue within a tissue explant allows for the study of drug transportation mechanisms and ways to improve drug penetration.

Compositions of Tissue Explants with Grafts

In some embodiments, the disclosure provides an ex vivo tissue composition comprising an isolated tissue explant from a source tissue and a population of cells or tissue providing one or more biological functions of normal or diseased cells or tissue, or one or more markers of said biological functions, within said tissue explant. Tissue explants described herein maintain features of in vivo tissue from which they are derived. Features include, without limitation, prolonged tissue expansion with cell proliferation, multilineage differentiation, and recapitulation of cellular and tissue architecture, including epithelial tissues, submucosal tissues, and stromal environments.

### A. Tissue Grafts

Described herein are populations of cells or tissues providing one or more biological functions of normal or diseased cells or tissues, or one or more markers of said biological functions. The populations of cells or tissues may be derived from a subject or cultured cells growth in vitro to mimic a tissue. In some embodiments, the population of cells or tissues are provided in any form suitable for placement within a tissue explant. In some embodiments, the term “graft” or “tissue graft” refers to said populations of cells or tissue. In some embodiments, the term “graft” or “tissue graft” refers to cells or tissues derived from a subject, e.g., a donor. In some embodiments, the term “graft” or “tissue graft” refers to a population of cells grown in vitro, for example, an immortalized cell line. In some embodiments, the term “graft” or “tissue graft” refers to an organoid formed in vitro. As used herein, the term “graft” or “tissue graft” is not limited to patient derived tissue as commonly used in medical terminology.

Tissue grafts are used in various methods of research and medical procedures and are developed from multiple tissue types and methods. In some aspects, the disclosure provides grafts within a tissue explant. In some embodiments, the graft is a xenograft. Xenografts are grafts derived from a donor that are different than the recipient species. In some embodiments, a xenograft comprises a population of cells or tissue from a first species (e.g., human) and is within a tissue explant of a second species (e.g., porcine). For example, in some embodiments, the tissue graft is derived from human tissue and the tissue explant is derived from porcine tissue. In some embodiments, the graft is an allograft. Allografts are grafts derived from a donor that is the same species as the recipient. In some embodiments, an allograft comprises a population of cells or tissue from a species and is within a tissue explant of the same species. For example, in some embodiments, the tissue graft and tissue explant are both derived from porcine tissue. In some embodiments, the graft is an autograft. Autografts are grafts derived from a donor from a first location and placed in the same donor at a second location. In some embodiments, an autograft comprises a population of cells or tissue from a first location of a mammal and is within a tissue explant from a second location of the mammal. For example, in some embodiments, the tissue graft comprises a population of cells or tissue from colon tissue of a mammal and is within a tissue explant from intestinal tissue of the same mammal. In some embodiments, the tissue graft and tissue explant is derived from the same type of tissue from the same species (e.g., both derived from intestinal tissue). In some embodiments, the tissue graft and tissue explant is derived from different types of tissue from the same species (e.g., the tissue explant is derived from intestinal tissue and the tissue explant is derived from non-intestinal tissue).

In some embodiments, the population of cells or tissue of the graft provide at least one biological function of healthy or diseased tissue, or expresses at least one marker of said biological function. In some embodiments, a biological function of healthy tissue is cell proliferation. In some embodiments, a biological function of diseased tissue is hyper cell proliferation. Methods for measuring cell proliferation are known to those of skill in the art, and include but are not limited to measuring marker Ki67.

In some embodiments, the graft is derived from primary tissue (e.g. connective tissue, epithelial tissue, muscle tissue, and/or nervous tissue). In some embodiments, the graft is derived from a mammal. In some embodiments, the graft is derived from a human. In some embodiments, the graft is derived from a large, non-human mammal. In some embodiments, the graft is derived from pigs, cows, goats, sheep, horses, donkeys, deer, antelopes and the like) and more generally, livestock (i.e., mammals raised for agricultural purposes such as pigs, cows, goats, sheep, horses, rabbits, and the link, and/or as beasts of burden such as donkeys, horses, elephants, camels, llamas, and the like). In some embodiments, the graft is derived from pig.

In some embodiments, the graft is derived from tissue of one or more of the duodenum, small intestine (jejunum and ileum), large intestine (colon), including the cecum, ascending colon, transverse colon, descending colon, and sigmoid colon, rectum, buccal tissue, lingual tissue, liver, pancreas, kidney, spleen, lung, heart, or skin.

In some embodiments, the graft comprises at least one population of cells associated with the gastrointestinal tract. In some embodiments, the graft comprises at least one population of cells associated with healthy gastrointestinal tissue. In some embodiments, the graft comprises at least one population of cells associated with diseased gastrointestinal tissue. In some embodiments, the graft comprises at least one population of cells associated with cancerous gastrointestinal tissue.

In some embodiments, the graft comprises at least one population of cells associated with liver tissue. In some embodiments, the graft comprises at least one population of cells associated with healthy liver tissue. In some embodiments, the graft comprises at least one population of cells associated with diseased liver tissue. In some embodiments, the graft comprises at least one population of cells associated with cancerous liver tissue. In some embodiments, the graft comprises hepatocytes.

In some embodiments, the graft comprises at least one population of cells associated with pancreas tissue. In some embodiments, the graft comprises at least one population of cells associated with healthy pancreas tissue. In some embodiments, the graft comprises at least one population of cells associated with diseased pancreas tissue. In some embodiments, the graft comprises at least one population of cells associated with cancerous pancreas tissue.

In some embodiments, the graft comprises at least one population of cells associated with kidney tissue. In some embodiments, the graft comprises at least one population of cells associated with healthy kidney tissue. In some embodiments, the graft comprises at least one population of cells associated with diseased kidney tissue. In some embodiments, the graft comprises at least one population of cells associated with cancerous kidney tissue.

In some embodiments, the graft comprises at least one population of cells associated with spleen tissue. In some embodiments, the graft comprises at least one population of cells associated with healthy spleen tissue. In some embodiments, the graft comprises at least one population of cells associated with diseased spleen tissue. In some embodiments, the graft comprises at least one population of cells associated with cancerous spleen tissue.

In some embodiments, the graft comprises at least one population of cells associated with lung tissue. In some embodiments, the graft comprises at least one population of cells associated with healthy lung tissue. In some embodiments, the graft comprises at least one population of cells associated with diseased lung tissue. In some embodiments, the graft comprises at least one population of cells associated with cancerous lung tissue.

In some embodiments, the graft comprises at least one population of cells associated with heart tissue. In some embodiments, the graft comprises at least one population of cells associated with healthy heart tissue. In some embodiments, the graft comprises at least one population of cells associated with diseased heart tissue. In some embodiments, the graft comprises at least one population of cells associated with cancerous heart tissue.

In some embodiments, the graft comprises at least one population of cells associated with skin tissue. In some embodiments, the graft comprises at least one population of cells associated with healthy skin tissue. In some embodiments, the graft comprises at least one population of cells associated with diseased skin tissue. In some embodiments, the graft comprises at least one population of cells associated with cancerous skin tissue.

In some embodiments, the graft is derived from tissue surgically resected from a subject. In some embodiments, the graft is derived from a tissue biopsy. Methods for surgically resecting tissue from a subject are known to those of skill in the art.

In some embodiments, the graft is derived from a cell line. In some embodiments, the graft comprises a population of cells of a cell line. In some embodiments, the cell line is a primary cell line (i.e. initiated from cells, tissues, or organ of an animal). In some embodiments, the cell line is an immortalized cell line (i.e. a cell line with an acquired mutation enabling indefinite proliferation).

In some embodiments, the graft is no bigger than the diameter of a well described herein. In some embodiments, the graft is smaller in diameter than the diameter of a well described herein.

In some embodiments the graft comprises healthy tissue. In some embodiments, the graft is derived from healthy tissue. In some embodiments, the graft comprises diseased tissue. In some embodiments, diseased tissue is generated ex vivo using the methods described herein. In some embodiments, the graft is derived from diseased tissue. In some embodiments, the diseased tissue is cancerous tissue. In some embodiments, the graft is derived from a neoplasia (e.g. a benign cell growth). In some embodiments, the graft is derived from a malignant neoplasia (e.g. a cancer). In some embodiments, the graft is derived from any stage tumor tissue. In some embodiments, the graft is derived from a stage 0, stage 1, stage IIA, stage IIB, stage IIC, stage IIIA (group 1), stage IIIA (group 2), stage IIIB (group 1), stage IIIB (group 2), stage IIIB (group 3), stage IIIC (group 1), stage IIIC (group 2), stage IIIC (group 3), stage IVA, or stage IVB cancer. In some embodiments, the graft is derived from a disease recurrence (e.g. cancer that returns after treatment). In some embodiments, the graft is derived from a refractory tumor (e.g. a tumor resistant to therapy).

In some embodiments, the graft is derived from a sporadic cancer. In some embodiments, the graft is derived from a hereditary cancer. Sporadic cancers are those which develop without inherited mutations and develop due to factors such as age, environment, and lifestyle choices (e.g. smoking). Hereditary cancers develop from cancer-causing mutations which are inherited (e.g. familial adenomatous polyposis which is inherited and increases an individual's risk of developing colon cancer).

In some embodiments, the graft is derived from a mammal of any age suitable as determined by one of ordinary skill in the art. In some embodiments, the graft is derived from a non-human mammal of any age suitable as determined by one of ordinary skill in the art. In some embodiments, the graft is derived from a mammal that is less than 1 year of age. In some embodiments, the graft is derived from a non-human mammal that is less than 1 year of age. In some embodiments, the graft is derived from a non-human mammal that is less than 1 month, less than 2 months, less than 3 months, less than 4 months, less than 5 months, less than 6 months, less than 7 months, less than 8 months, less than 9 months, less than 10 months, less than 11 months, or less than 1 year of age. In some embodiments, the graft is derived from a non-human mammal less than 6-months of age. In some embodiments, the graft is derived from a pig less than 6-months of age.

i. Organoids

In some embodiments, a graft placed within a tissue explant is an organoid. Organoids are currently applied to general biology and disease research, and are applicable to studying general physiology or other methods such as screening drug response due to their ability to recapitulate organ like functions in vitro.

Organoids are generated using various methods known in the art. In some embodiments, the organoid is derived from primary tissue. In some embodiments, the organoid is derived from a tissue biopsy. In some embodiments, the organoid is derived from the same subject as the tissue explant. In some embodiments, the graft is an enteroid (i.e. a class of organoids generated from the small intestine).

In some embodiments, the graft is an intestinal organoid. Intestinal organoids are known to those of skill in the art (e.g., US 20100047853) and described herein. In some embodiments, the graft is a liver organoid. Liver organoids have been described and are known to those of skill in the art (e.g., US20130189327A1 and US20190314387A1). In some embodiments, the graft is a pancreatic organoid. Pancreatic organoids have been described and are known to those of skill in the art (e.g., US20200188443A1). In some embodiments, the graft is a cardiac organoid. Cardiac organoids have been described and are known to those of skill in the art (e.g., US20210017496A1 and US20170002330A1). In some embodiments, the graft is a skin organoid. Skin organoids have been described and are known to those of skill in the art (e.g., US20180305671A1 and US20200131482A1). In some embodiments, the graft is a spleen organoid. Spleen organoids have been described and are known to those of skill in the art (e.g., Gee, K et al. 2020 Tissue Eng. Part A. 26(7-8):411-418). In some embodiments, the graft is a kidney organoid. Kidney organoids have been described and are known to those of skill in the art (e.g., US20160361466A1 and US20200291361A1). In some embodiments, the graft is a lung organoid. Lung organoids have been described and are known to those of skill in the art (e.g., US20200283735A1 and US20180201350A1).

In some embodiments, the organoid is derived from a mammal of a suitable age as determined by one of ordinary skill in the art. In some embodiments, the organoid is derived from a mammal that is less than 1 year of age. In some embodiments, the organoid is derived from a non-human mammal that is less than 1 year of age. In some embodiments, the organoid is derived from a non-human mammal that is less than 1 month, less than 2 months, less than 3 months, less than 4 months, less than 5 months, less than 6 months, less than 7 months, less than 8 months, less than 9 months, less than 10 months, less than 11 months, or less than 1 year of age. In some embodiments, the organoid is derived from a non-human mammal less than 6-months of age. In some embodiments, the organoid is derived from a pig less than 6-months of age.

In some embodiments, the organoid is derived from a non-human mammal that is no more than 100 kg in weight. In some embodiments, the organoid is derived from a pig that is no more than 100 kg in weight. In some embodiments, the organoid is derived from a non-human mammal that is no more than 10 kg, 20 kg, 30 kg, 40 kg, 50 kg, 60 kg, 70 kg, 80 kg, 90 kg, or 100 kg in weight. In some embodiments, the organoid is derived from a non-human mammal that is no more than 70 kg in weight. In some embodiments, the organoid is derived from a pig that is no more than 70 kg in weight.

Organoids are derived from various cell and tissue types including primary cells, stem cells, and reprogrammed cells (see, e.g. Miura and Suzuki, 2018 Dev. Growth & Differentiation. 60(6); Spence et al. 2011 Nature 470: 105-109). In some embodiments, the organoid is derived from an immortalized cell line. In some embodiments, the organoid is differentiated from stem cells. Cells taken directly from live tissue, i.e. freshly isolated cells, are also referred to as primary cells. In some embodiments the epithelial stem cells are primary epithelial stem cells. In some embodiments, the epithelial stem cells are (or are derived from) primary epithelial stem cells. In some embodiments, the differentiated organoid comprises epithelial cells.

In some embodiments, the organoid is a three-dimensional organoid, comprising crypt-like domains surrounding a central lumen, and contain intestinal stem cells that are polarized, residing in the bases of the structures that can actively divide and give rise to all major differentiated cell lineages present in the intestine.

Various methods exist for generating organoids from different cell and tissue sources. These methods are known to those of ordinary skill in the art. In some embodiments, cells for generating an organoid are isolated by collagenase digestion, for example, as described in the examples and in Dorell et al., 2008 (Hepatology. 2008 October; 48(4):1282-91). In some embodiments, collagenase digestion is performed on a tissue biopsy. In some embodiments, collagenase and accutase digestion are used to obtain the epithelial stem cells for use in generating an organoid. Following tissue digestion, cells are suspended in culture medium and matrix/scaffold.

Natural and synthetic matrix/scaffolds enable three-dimensional growth of organoids. Example matrix/scaffolds include, but are not limited to Matrigel, alginate, nanofibrillar cellulose, collagen, fibrin, and/or polyethylene glycol, among others.

The culture period for generating organoids is not limited and can be appropriately adjusted by those of skill in the art. In some embodiments, the organoids are established in culture for 2-months. In some embodiments, the organoids are established in culture for 1 week to 2-months. In some embodiments, the organoids are cultured for 1 to 60 days to form a three-dimensional structure. In some embodiments, the organoids are established in culture for 1 day, 5 days, 10 days, 15 days, 20 days, 25 days, 30 days, 35 days, 40 days, 45 days, 50 days, 55 days, or 60 days. In some embodiments, the organoids are passaged while in culture. The skilled person would know how to split the organoids in order to passage them so that they can multiply. In some embodiments, the organoids are frozen until they are cultured prior to placement within the tissue explant.

Certain advance tumors display loss of their apical-basal polarity. Loss of polarity in tumor cells is known to contribute to metastasis and progression of disease. In some embodiments, the organoids have reversal of apical-basal polarity.

In some embodiments, the organoids are about 10 μm to about 550 μm in diameter. In some embodiments, the organoids are about 10 μm, about 50 μm, about 100 μm, about 150 μm, about 200 μm, about 250 μm, about 300 μm, about 350 μm, about 400 μm, about 450 μm, or about 500 μm in diameter. In some embodiments, organoids are smaller than 500 μm in diameter. In some embodiments, the organoid is no bigger than the diameter of a well described herein. In some embodiments, the organoid is smaller in diameter than the diameter of a well described herein.

In some embodiments, one or more cells used to generate an organoid are gene-edited. In some embodiments, one or more cells used to generate an organoid are gene-edited prior to forming an organoid. For example, stem cells used to form an organoid as described herein are gene-edited prior to addition of exogenous factors needed to form an organoid (e.g., growth factors). Exogenous factors needed to form an organoid differ based on the type or organoid being from and are known to those of skill in the art. In some embodiments, one or more cells are gene-edited after formation of an organoid. For example, where an organoid is formed from intestinal crypts, the crypts are formed into an organoid and then gene edited. In some embodiments, one or more cells are gene-edited while forming an organoid. In some embodiments, one or more cells are gene-edited before formation of an organoid. Any of the gene editing tools known to those of skill in the art and/or described herein are used for overexpression or reduced/inhibited expression of one or more genes in the organoid, e.g. by enhancing a promoter of an oncogene. Any of the gene editing tools known to those of skill in the art and/or described herein are used for or disruption, ablation or inhibited expression of one or more tumor suppressor genes.

In some embodiments, the organoid is gene-edited using CRISPR/Cas9, which comprises the use of guide RNA (gRNA) or single guide RNA (sgRNA) as described in detail below. In some embodiments, gRNA and/or sgRNA are cloned into PC458 expression vector (Addgene). In some embodiments, the gRNA and/or sgRNA is incorporated into lentivirus. In some embodiments, organoids are gene-edited to knock-out adenomatous polyposis coli (APC) gene. In some embodiments, the sgRNA having the nucleotide sequence set forth in any one or more of SEQ ID NOs: 1-8 are used to knock-out the APC gene in organoids. In some embodiments, organoids are gene-edited to knock-out tumor protein 53 (p53). In some embodiments, the sgRNA having the nucleotide sequence set forth in any one or more of SEQ ID NO: 9-16 is used to knock-out the p53 gene in organoids. In some embodiments, organoids are gene edited to knock-out mothers against decapentaplegic homolog 4 (SMAD4) gene. In some embodiments, the sgRNA having the nucleotide sequence set forth in any one or more of SEQ ID NO: 17-24 is used to knock-out the SMAD4 gene in organoids. In some embodiments, the organoids are gene-edited to knock-out one or more of APC, P53, and SMAD4. In some embodiments, the organoid placed in the tissue explant is at least APC⁻/P53⁻/SMAD4⁻.

ii. Gene-Editing Tissue Grafts

In some embodiments, the graft comprises at least one population of gene-edited cells. Gene-editing provides the ability to generate models harboring mutations similar to those found in diseased tissue (e.g. a tumor). In some embodiments, a wild-type graft is gene-edited to induce a disease state in the graft. In some embodiments, a graft derived from diseased tissue is gene-edited to correct the disease state in the graft. In some embodiments, a graft derived from diseased tissue is gene-edited to introduce additional disease inducing mutations. By way of non-limiting example, tissue and/or cells of the graft are genetically modified to increase or decrease gene expression or to express an exogenous gene (e.g. a marker gene). Methods of genetically modifying tissue and/or cells are well known in the art and can include, but are not limited to, viral vectors, plasmid vectors, homologous recombination, stable integration, and transient expression. Methods and tools for gene-editing include, but are not limited to the CRISPR endonuclease system, CRISPR/Cas9, Zinc Finger Nucleases, Transcription Activator-Like Effector Nucleases (TALENs), Homing Endonucleases, RNA-Guided Endonucleases, guide RNAs, non-homologous end joining, and homology-directed repair. In some embodiments, the gene edit knocks-out, over-expresses, or mutates a gene. In some embodiments, the gene-edit reduces gene expression. In some embodiments, the gene-edit increases gene expression. In some embodiments, the gene-edit causes expression of a mutated form of the gene (e.g. a genetic knock-in).

The CRISPR gene editing system is an RNA-guided DNA-targeting platform. The CRISPR/Cas system uses short guide RNAs (gRNA) to direct a Cas nuclease to a genomic region of interest. The gRNA guides precise cleavage by the nuclease at the region of interest. In some instances, a CRISPR nuclease may be expressed from a plasmid or integrated into a host genome. Various methods of performing CRISPR/Cas9 mediated genome modification, including the conditions permissive for CRISPR/Cas9 mediated homology-directed repair in various settings, including in vivo and in vitro settings include are known to those of skill in the art.

In some embodiments, the graft comprises at least one mutation introduced into the graft by gene-editing. In some embodiments, the graft has one, two, three, four, five, or six gene-edits. In some embodiments, the gene-edits induce the graft to develop diseased tissue (e.g. a tumor). For example, using recombinant DNA techniques, a gene that expresses a product (e.g., wild-type or mutated protein) that is associated with a disease or disorder is placed under the control of a viral or tissue-specific promoter. The recombinant DNA construct containing the gene is used to transform or transfect one or more cells of the graft. The graft which expresses the introduced gene or gene product can then be studied for disease or disorder progression or for the effectiveness of treatments against the particular disease or disorder. Additionally, gene editing techniques are used to modify the genes and gene products made by the graft. For example, using CRISPR/Cas system transforms the graft to model a specific disease. The introduction of genes and gene products, or mutations thereof, into the graft can happen at any stage of formation or culture of the graft, e.g., the genes and gene products are introduced into undifferentiated immature cells used to generate a graft, or to very differentiated mature graft tissue, as is further taught herein.

In some embodiments, one or more of the following genes are edited in at least one population of cells in a graft: ABL1, ACO1, ACVR2A, AFP, AKT1, ALK, ALPPL2, ANAPC1, APC, ARID1A, AR, AR-v7, ASCL2, β2M, BRAF, BTK, C150RF40, CDH1, CLDN6, CNOT1, CT45A5, CTAG1B, DCT, DKK4, EEF1B2, EEF1DP3, EGFR, EIF2B3, EPHB2, ERBB3, ESR1, ESRP1, FAM11 IB, FGFR3, FRG1B, GAGE1, GAGE 10, GATA3, GBP3, HER2, IDH1, JAK1, KIT, KRAS, LMAN1, MABEB 16, MAGEA1, MAGEA10, MAGEA4, MAGEA8, MAGEB 17, MAGEB4, MAGEC1, MEK, MLANA, MLL2, MMP13, MSH3, MSH6, MYC, NDUFC2, NRAS, PAGE2, PAGE5, PDGFRa, PIK3CA, PMEL, pol protein, POLE, PTEN, RAC1, RBM27, RNF43, RPL22, RUNX1, SEC31A, SEC63, SF3B 1, SLC35F5, SLC45A2, SMAP1, SMAP1, SPOP, TFAM, TGFBR2, THAP5, TP53, TTK, TYR, UBR5, VHL, XPOT, JAK2, IDH1, CTNNB1, NPM1, CALR, FGFR3, CDKN2A, KIT, MYD88, HRAS, MED12, DNMT3A, GNAS, IDH2, KCNJ5, PTEN, NOTCH1, SF3B1, FLT3, ASXL1, SRSF2, FOXL2, PTPN11, GNAQ, RET, HLA-A, MPL, IKZF1, KMT2C, TET2, PDGFRA, FBXW7, H3F3A, ALK, CEBPA, ESR1, AKT1, RUNX1, GNA11, VHL, WT1, U2AF1, ABL1, ERBB2, DICERI, NOTCH4, EZH2, HNF1A, SMARCB1, CXCR4, PLCG1, TSHR, PRKACA, RHOA, STAT3, POLE, SETBP1, MET, AR, STK11, NF2, CBL, HLA-B, PRKCB, ATR, PPP2R1A, CASC5, CD79B, PBRM1, PTK2B, GATA2, KMT2D, SULTIA1, FLNB, PRPF8, RNF43, MSH6, FGFR2, SMAD4, JAK3, USP8, DLC1, ESRP1, LRP1B, MYH11, BRCA1, CARD11, HSP90AB1, MAP3K9, ADAMTSL3, PDGFRB, RPTOR, ROS1, NFKBIE, AMER1, KLF4, RAC1, TERT, MYOD1, ATP1A1, CSF3R, NOTCH2, CCR4, PAX5, SPTAN1, MLH1, CUBN, RNF213, SMO, ABCC4, AXIN2, CSF1R, PER1, PKHD1, IL7R, RB1, ARID1A, ATM, FES, MTHFR, PTCH2, FANCI, CDH5, CIC, IL6ST, MYH9, NF1, TGFBR2, INSR, PTPN12, TNFAIP3, MEN1, NSD1, SLITRK6, SYT1, TNKS, CCND3, PSMD13, CYP2D6, HELQ, LPHN3, PRAME, STAT5B, BCL6, CCDC6, CCND1, FLCN, LMO2, MUC1, NFKBIZ, NRP2, CTCF, HIST1H3B, KEAP1, SLC22A2, ABCC2, EED, GATA1, GLI3, IKZF3, PIK3CG, XPO1, CHRNA3, MAP2K1, SETD2, ZNF668, CCND2, FLT4, NT5C2, RECQL4, SSX1, ALOX12B, CDKN1B, ELF3, INPP4B, MARVELD3, MLLT4, MLPH, NTRK3, SPOP, BCL2, EPHB1, ERCC4, ERCC6, ETNK1, JAK1, LRP2, MUTYH, NFKBIA, ARNT, BRCA2, and CDH2.

In some embodiments, SMAD family member 4 (SMAD4) is gene edited in the tissue graft. SMAD4 is a signal transduction protein and regulates TGF-β signaling. SMAD4 is activated in part by TGF-β and recognizes smad-binding elements on DNA. DNA binding induces expression of TGF-β target genes. The TGF-β/SMAD4 signaling pathway functions as a growth regulator by inducing cell cycle arrest and apoptosis. Because of this function, SMAD4 is considered a tumor suppressor. Loss of, or mutations in this protein may result in cancer.

In some embodiments, adenomatous polyposis coli (APC) is gene edited in the tissue graft. APC is a tumor suppressor and functions in regulating β-catenin. When APC binds to β-catenin it leads to the degradation and eventual down-regulation of β-catenin. Without APC, β-catenin signaling goes unchecked which results in hyperproliferation of cells and eventually cancer.

In some embodiments, tumor protein 53 (p53) is gene edited in the tissue graft. p53 is one of the most widely known cancer genes. The p53 protein controls multiple cell functions including cell cycle arrest, apoptosis, and DNA repair among others. Loss of, or mutations in this protein may result in cancer.

In some embodiments, cells in a tissue graft are edited using CRISPR/Cas9, which comprises the use of guide RNA (gRNA) or single guide RNA (sgRNA) as described in detail below. In some embodiments, gRNA and/or sgRNA is cloned into PC458 expression vector (Addgene). In some embodiments, the gRNA and/or sgRNA is incorporated into lentivirus. In some embodiments, the cells in a tissue graft are gene-edited to knock-out adenomatous polyposis coli (APC) gene. In some embodiments, the sgRNA having the nucleotide sequence set forth in any one or more of SEQ ID NO: 1-8 is used to knock-out the APC gene in the graft. In some embodiments, the graft is gene-edited to knock-out tumor protein 53 (p53). In some embodiments, the sgRNA having the nucleotide sequence set forth in any one or more of SEQ ID NO: 9-16 is used to knock-out the p53 gene in the graft. In some embodiments, the graft is gene edited to knock-out mothers against decapentaplegic homolog 4 (SMAD4) gene. In some embodiments, the sgRNA having the nucleotide sequence set forth in any one or more of SEQ ID NO: 17-24 is used to knock-out the SMAD4 gene in the graft. In some embodiments, the graft is gene-edited to knock-out one or more of APC, P53, and SMAD4. In some embodiments, the graft placed in the tissue explant is at least APC⁻/P53⁻/SMAD4⁻.

In some embodiments, to visualize the grafts described herein after being placed in a tissue explant, cells in the graft are gene-edited to express a fluorescent marker. In some embodiments, the graft is gene edited to express a fluorescent protein. In some embodiments, the fluorescent protein is selected from but not limited to GFP, RFP, YFP, or BFP. In some embodiments, the graft is transduced with lentivirus to express a fluorescent marker. Methods known in the art such as fluorescence microscopy are used to measure growth of the graft, cell death, and/or cell penetration of the graft (e.g. metastasis) after placement within the tissue explant.

iii. Gastrointestinal Cancer Grafts

In some embodiments, the graft is derived from gastrointestinal tissue. In some embodiments, the graft is derived from a gastrointestinal tumor. In some embodiments, the graft is derived from a sessile serrated lesion (e.g. a premalignant flat lesion of the colon). In some embodiments, the graft is derived from a sporadic colorectal cancer. In some embodiments, the graft is derived from an inherited colorectal cancer syndrome (e.g., Familial Adenomatous Polyposis or Lynch Syndrome). While common mutations are known in CRC (e.g. APC, KRAS, and P53), tumors may harbor any number and type of genetic or epigenetic modifications. In some embodiments, the graft comprises any combination of genetic or epigenetic alterations.

Various genetic mutations give rise to colon tumors. Colon tumors develop from one or more genetic mutations or genome instabilities. In some embodiments, the graft is derived from healthy colon tissue and gene edited to express one or more known colon cancer mutations. In some embodiments, the graft is derived from healthy colon tissue and gene edited to comprise a gene expression profile (e.g., knocked-down genes) associated with colon cancer. In some embodiments, the gene-edited graft forms a tumor. In some embodiments, the gene-edit induces formation of a tumor and/or cancerous cells within the graft. In some embodiments, one or more of the following genes are edited in at least one population of cells in a graft: ABL1, AKT1, ALK, APC, AR, ATM, BRAF, CDH1, cKIT, cMET, CSF1R, CTNNB1, EGFR, ER, ERBB2, ERBB4, FBXW7, FGFR1, FGFR2, FLT3, GNA11, GNAQ, GNAS, HER2, HNF1A, HIF2A, HRAS, IDH1, JAK2, JAK3, KDR (VEGFR2), KRAS, MGMT, MLH1, MPL, NOTCH1, NPM1, NRAS, PDGFRA, PGP, PIK3CA, PR, PTEN, PTPN11, RB1, RET, RRM1, SMAD4, SMARCB1, SMO, SPARC, STK11, TLE3, TOP2A, TOPO1, TP53, TS, TUBB3, and VHL.

In some embodiments, the graft is an intestinal organoid. In some embodiments, the intestinal organoid comprises primary cells. In some embodiments, the intestinal organoid comprises cells of an immortalized cell line. In some embodiments, the intestinal organoid expresses at least one marker associated with intestinal tissue. In some embodiments, the at least one marker associated with intestinal tissue is selected from CDX2, Muc2, and Lgr5. In some embodiments, an intestinal organoid comprises stem cells that reside in in vivo crypts in intestine. In some embodiments, an intestinal organoid comprises intestinal stem cells and differentiated epithelial progeny. In some embodiments, an intestinal organoid comprises all intestinal cell types described herein, such as Lgr5+ stem cells, goblet cells, and enteroendocrine cells. In some embodiments, an intestinal organoid comprises a crypt domain and a villus domain. In some embodiments, an intestinal organoid comprises a polarized epithelial layer surrounding a functional lumen. In some embodiments, an intestinal organoid recapitulates at least one function of the intestine. In some embodiments, an intestinal organoid is cancerous and is capable of forming tumors when implanted in vivo. In some embodiments, a cancerous intestinal organoid comprises at least one population of cells comprising at least one genetic mutation. In some embodiments at least one genetic mutation is associated with cancer. In some embodiments, the at least one genetic mutation is endogenous to the population of cells. In some embodiments, the at least one genetic mutation is introduced via gene-editing to the population of cells.

In some embodiments, the graft is a colon organoid (i.e., colonoid). In some embodiments, the colon organoid comprises primary cells. In some embodiments, the colon organoid comprises cells of an immortalized cell line. In some embodiments, a colon organoid comprises stem cells that reside in in vivo crypts in colon. In some embodiments, the colon organoid recapitulates at least one function of the colon. In some embodiments, a colon organoid is cancerous and is capable of forming tumors when implanted in vivo. In some embodiments, a cancerous colon organoid comprises at least one population of cells comprising at least one genetic mutation. In some embodiments at least one genetic mutation is associated with cancer. In some embodiments, the at least one genetic mutation is endogenous to the population of cells. In some embodiments, the at least one genetic mutation is introduced via gene-editing to the population of cells.

### B. Composition of Tissue Explant

In some embodiments, the isolated tissue explant is derived from a source tissue. In some embodiments, the source tissue is an organ. In some embodiments, the source tissue has an architecture that is maintained in the tissue explant. In some embodiments, the source tissue and the tissue explant have the same architecture. In some embodiments, the source tissue is any tissue within a mammal (human or non-human). In some embodiments, the source tissue is any tissue within the gastrointestinal tract. In some embodiments, the source tissue is liver tissue. In some embodiments, the source tissue is heart tissue (i.e. cardiac tissue). In some embodiments, the source tissue is pancreatic tissue. In some embodiments, the source tissue is splenic tissue. In some embodiments, the source tissue is kidney tissue (i.e. renal tissue). In some embodiments, the source tissue is skin tissue.

i. Gastrointestinal Tissue

In some embodiments, the tissue explant is derived from a tissue within a gastrointestinal tract. In some embodiments, the tissue explant is derived from intestinal tissue. Tissue explants derived from intestinal tissue have been described in the art (see e.g. US20190064153A1). In some embodiments, the tissue explant is derived from colon tissue. In some embodiments, the tissue explant is derived from stomach tissue. In some embodiments, the tissue explant is derived from esophagus tissue. In some embodiments, the tissue explant is derived from buccal tissue. In some embodiments, the tissue explant is derived from lingual tissue. In some embodiments, the tissue explant is derived from rectum tissue.

a. Intestine

Like other parts of the gastrointestinal tract, the small intestine is comprised of four basic layers: the mucosa, submucosa, muscularis externa, and serosa. It is the body's major digestive organ, the site where digestion is completed and almost all absorption occurs. The small intestine is highly adapted for nutrient absorption. Both its long length and the modifications of its inner surface provide an extraordinary large surface area and enhance absorption enormously.

The outermost layer of the intestine, the serosa, is a smooth membrane consisting of a thin layer of cells that secrete serous fluid, and a thin layer of connective tissue. The muscularis externa, adjacent to the submucosa membrane, comprises two muscle layers of an inner circular and outer longitudinal smooth muscle. It is responsible for gut movement (i.e., peristalsis). The submucosa is a layer of dense irregular connective tissue or loose connective tissue that supports the mucosa and joins it to the underlying smooth muscle. The innermost layer and lining of the small intestine is the mucosa. It is a mucous membrane that secretes digestive enzymes and hormones. The mucosa comprises intestinal villi, an epithelium and a lamina propria. The lamina propria is a thin layer of loose connective tissue, or dense irregular connective tissue, which lies beneath the epithelium and together with the epithelium constitutes the mucosa.

In some embodiments, the tissue explant described herein comprises the serosa, muscularis externa, submucosa and mucosa. In some embodiments, the tissue explant described herein comprises the muscularis externa, submucosa and mucosa. In some embodiments, the tissue explant described herein comprises the inner circular smooth muscle, the submucosa, and the mucosa. Methods for identifying these structures include visual inspection, by, for example, histological staining (e.g., haemotoxylin & eosin stain) followed by microscopic analysis. Using such methods, one of skill in the art can identify the various structures of the tissue explant.

In some embodiments, the tissue explant described herein comprises a fully intact extracellular matrix. In some embodiments, the extracellular matrix comprises the lamina propria. In some embodiments, the extracellular matrix comprises the lamina muscularis.

In some embodiments, the tissue explant described herein maintains polarity (e.g., epithelial cell polarity) as described herein. In some embodiments, the tissue explant described herein is in a planar position, thereby providing a luminal surface and a basolateral surface. In some embodiments, either surface is accessible. Methods of determining polarity are known to those of skill in the art. A review of such methods can be found in Chapter 7 of Cell Polarity and Morphogenesis (Academic Press, 2017, herein incorporated by reference in its entirety). In some embodiments, polarity of the tissue explant described herein is analyzed by visual (e.g., microscopic) inspection. For example, since the tissue explant described herein comprises two or more genetically distinct cell populations, polarity can be determined by expression of a labeled protein in only a subset of cells and subsequently visualized by microscopic techniques. In some embodiments, immunohistochemistry and live images of fluorescent reports are used to visualize proteins in their tissue context and evaluate their distribution. In some embodiments, cell polarization is quantified by analyzing protein localization in fluorescent images and calculating the ratio of fluorescence intensity between regions where the protein is present and regions where it is weakly localized or absent. The fluorescence ratio provides a quantitative measure of asymmetric protein distribution. See Marcinkevicius, E., et al. J. Biol. 2009, Vol. 8(12): 103, herein incorporated by reference in its entirety. In some embodiments, the fluorescence ratio is normalized by choosing appropriate analysis settings and incorporating internal controls, as described by Shimoni, R., et al. PLos ONE 2014, Vol. 9(6): e99885, herein incorporated by reference in its entirety.

In some embodiments, the tissue explant described herein maintains the in vivo architecture of the intestinal tissue from which it is derived. In some embodiments, the in vivo architecture is determined by visual inspection by methods known to those of skill in the art and described infra. For example, in some embodiments, determination of the maintenance of the in vivo architecture comprises comparing freshly excised tissue with tissue explants cultured ex vivo over time. In some embodiments, the tissue explant comprises intestinal epithelium from a source tissue, wherein said source tissue comprises an architecture and wherein the tissue explant comprises said architecture. In some embodiments, the architecture of the source tissue comprises epithelial cells having a polarity. In some embodiments, the architecture of the source tissue comprises a circular muscle layer. In some embodiments, the architecture of the source tissue comprises intestinal villi. In some embodiments, the architecture of the source tissue comprises an intact extracellular matrix. In some embodiments, the architecture of the source tissue comprises an intact extracellular matrix comprising lamina propria and/or lamina muscularis. In some embodiments, the architecture of the source tissue comprises epithelial cells having a polarity and a circular muscle layer. In some embodiments, the architecture of the source tissue comprises epithelial cells having a polarity, a circular muscle layer, and intestinal villi.

The intestinal villi, fingerlike extensions of the inner mucosal surface, are one of the primary specializations characteristic of the intestine's absorption and digestion functions. The epithelial cells that comprise the villi are chiefly absorptive cells or enterocytes. Their capacity to secrete, absorb, and digest specific ions and nutrients, depends on their position along the length of the intestine. The enterocytes, themselves, have microvilli, giving the mucosal surface a fuzzy appearance sometimes called the “brush border.” The microvilli comprise enzymes which aid in digestion, such as disaccharidases and peptidases. In some embodiments, the tissue explant described herein comprises enterocytes. In some embodiments, enterocytes are identified by the presence of villin, e-cadherin, keratin 20, and/or fatty acid binding protein 1 (FABP1). In some embodiments, the tissue explant described herein comprises villi.

The intestinal mucus layer plays an important protective role. The mucus layer is primarily comprised of mucins. Mucins are highly glycosylated large glycoproteins with protein backbone structures rich in serine and threonine, which are linked to a wide variety of O-linked oligosaccharide side chains that make up more than 70% of the weight of the molecule. Up to 20 different mucin genes have been identified, MUC1 to MUC20 according to order of their discovery. Mucin genes are expressed in tissue and cell type-specific manner and are broadly classified into two types, secretory and membrane-associated. In small and large intestine, MUC2 is the major secretory mucin synthesized and secreted by goblet cells. Intestinal mucus layers secreted by goblet cells consist mainly of compact mesh-like network of viscous, permeable, gel-forming MUC2 mucin, which provides the frontline host defense against endogenous and exogenous irritants and microbial attachment and invasion but allows the transport of nutrients. In some embodiments, the tissue explant comprises mucin secreting goblet cells. In some embodiments, the tissue explant forms a mucus layer in culture. In some embodiments, the tissue explant described herein comprises mucosubstances. In some embodiments, the mucosubstances are glycoproteins, glycolipds or mucins.

Mucin 2 (Muc 2) as well as Caudal type homeobox 2 (CDX2) are both markers for the mucin secreting goblet cells within the intestinal epithelium. In some embodiments, goblet cells are identified by the presence of Mucin 2 (Muc 2) and/or Caudal type homeobox 2 (CDX2).

In some embodiments, presence of a mucus layer in the tissue explant described herein is determined by measuring the presence of mucins and/or mucosubstances. In some embodiments, the presence of a mucus layer in the tissue explant described herein is determined by measuring the gene expression of Muc 2 and/or CDX2. In some embodiments, the presence of a mucus layer in the tissue explant described herein is determined by measuring the protein expression of Muc 2 and/or CDX2. In some embodiments, the presence of a mucus layer in the tissue explant described herein is determined by visual inspection (e.g., microscopy). In some embodiments, histological staining, such as with alcian blue tissue stain, is used for visual inspection.

Between the villi, the mucosa is studded with pits or openings which lead into tubular intestinal glands called intestinal crypts or crypts of Lieberkuhn. The epithelial cells which line the crypts secrete intestinal juice, a fluid mixture comprising mucus. Deep in the crypts are Paneth cells which produce various polypeptides, such as cryptdin, lysozyme, type II (secretory) phospholipase A2, intestinal defensin (e.g., RIP-3). In some embodiments, the tissue explant described herein comprises intact crypts. In some embodiments, intact crypts are identified by visual inspection (e.g., microscopy). Methods of visual inspection for identifying intact crypts include, but are not limited to, histological tissue staining and normal light microscopy.

The gastrointestinal tract is characterized by self-renewing epithelium fueled by adult stem cells residing at the bottom of the intestinal crypt and gastric glands. In the adult intestine, cellular division only occurs in the crypt, not in the villus. Several potential stem cell populations have been proposed in the crypt. One of them, named crypt based columnar (CBC) cells is closely associated with Paneth cells at crypt bottoms. CBCs along with Paneth cells have long been proposed to form a restricted stem cell zone within the crypt, which has been confirmed by lineage tracing experiments. Such lineage tracing experiments have revealed that single Lgr5+(leucine-rich repeat-containing G-protein coupled receptor 5) CBC cells are able to regenerate an entire crypt-villus axis. These cells are in a state of “stemness” and possess long-term self-renewal capabilities as well as multipotent differentiation abilities. In some embodiments, the tissue explant described herein comprises intestinal stem cells. In some embodiments, the intestinal stem cells are Lgr5+. In some embodiments, the presence of intestinal stem cells in the tissue explant described herein is responsible for long-term maintenance of the explant.

In addition to Lgr5+, olfactomedin-4 (OLFM4) emerged as a robust marker for intestinal stem cells based on a gene signature of Lrg5 stem cells. Therefore, in some embodiments, the tissue explant described herein comprises OLFM4+ stem cells. In some embodiments, the tissue explant described herein comprises Lrg5+ and OLFM4+ stem cells. In some embodiments, Lrg5+ and OLFM4_stem cells are detected by methods known to those of skill in the art and further described herein.

Several signaling mechanisms are also involved in maintaining the renewal capacity of the small intestine. Wnt, BMP/TGF-0, Notch and EGF are key regulators of epithelial homeostasis and self-renewal activity. While the cells move across the crypt-villus axis they are exposed to a Wnt gradient. Stem cells become loaded with Wnt mediators that are produced by adjacent Paneth cells, which bind to their cognate Frizzled receptors. Due to their local production and limited diffusion, Wnt molecules as well as their receptors are diminished through turnover by cellular division as the cells leave the stem cell zone and move away from Paneth cells. Besides Lgr5+, the CBC stem cells express a whole set of further Wnt pathway associated genes, which directly controls sternness in the intestinal crypts. The high Wnt activity in CBC stem cells is mediated by binding of secreted R-spondin family members to Lgr family members on the CBC membrane. This binding potentiates the Frizzled mediated Wnt pathway activation and results in robust activation of the Wnt pathway. Moreover, myofibroblasts play a role in maintaining the renewal capacity of the small intestine by providing signaling cues. Specifically, myofibroblasts, which surround the intestinal crypt, secrete factors such as Wnt ligands, HGF, BMP and Noggin, important in regulating differentiation (see Medema, J. and Vermeulen, L., Nature, Vol. 474: 318-326, 2011, herein incorporated by reference).

Prior intestinal model systems, including primary intestinal epithelial cells and/or intestinal stem cells, require exogenous addition of Wnt to maintain the systems. The tissue explants described herein do not require exogenous Wnt for culture maintenance. The presence of intact crypts and villi, along with stroma, contribute to this feature of the tissue explants described herein.

In some embodiments, the tissue explant described herein comprises intestinal endocrine cells. Intestinal endocrine cells, or enteroendocrine cells, are restricted to the mucosa and located within the intestinal crypts and villi (Moran, G., et al. Therap Adv Gastroenterol. 2008 July; Vol. 1(1): 51-60, herein incorporated by reference in its entirety). Enteroendocrine cells found in the small intestine include, but are not limited to, cholecystokinin-secreting cells, secretin-secreting S cells, gastric inhibitory polypeptide-secreting cells, motilin-secreting M cells and neurotensin secreting N cells, and neuroendocrine L cells. In some embodiments, the tissue explant described herein comprise L cells. Enteroendocrine cells are characterized by the presence of secretary vesicles. Enteroendocrine cells secrete glucagon-like peptide-1 (GLP-1). In some embodiments, secretion of GLP-1 is in response to the presence of glucose. In some embodiments, secretion of GLP-1 is in response to the presence of acetylcholine. In some embodiments, secretion of GLP-1 is in response to the presence of LiCl. In some embodiments, secretion of GLP-1 is determined by the concentration of GLP-1 7-36. In some embodiments, the tissue explant described herein is responsive to glucose, acetylcholine and/or LiCl due to the presence of enteroendocrine cells.

In some embodiments, the tissue explant described herein comprises tight junctions. In some embodiments, tight junctions are identified by the presence of claudin-1, e-cadherin, or a combination thereof, determined by methods known to those of skill in the art and further described herein. Claudin-1 is an integral membrane protein and e-cadherin is a transmembrane protein, both of which are components of tight junctions. Tight junctions represent one mode of cell-to-cell adhesion in epithelial or endothelial cell sheets, forming continuous seals around cells and serving as a physical barrier to prevent solutes and water from passing freely.

The submucosa contains individual and aggregated lymphoid patches, the latter called Peyer's patches. In the duodenum only, mucus-secreting duodenal glands (also called Brunner's glands) are found. Microfold (M) cells are found in Peyer's patches of the intestine and are specialized for the phagocytosis and transcytosis of gut lumen macromolecules. These cells play an important role in the induction of specific mucosal immune responses in the Peyer's patches, and allow for transport of microbes and particles across the epithelial cell layer from the gut lumen to the lamina propria where interactions with immune cells can take place. In some embodiments, the tissue explant described herein comprises microfold cells. Microfold cells are identified by cytoskeletal and extracellular matrix components expressed at the edge of the cells or on their cell surfaces, including actin, villin, cytokeratin and vimentin. In some embodiments, microfold cells are identified by the presence of vimentin, actin, cytokeratin, villin, or combination thereof. In some embodiments, microfold cells are identified by the presence of vimentin. In some embodiments, microfold cells are identified by the presence of actin. In some embodiments, microfold cells are identified by the presence of villin. In some embodiments, microfold cells are identified by the presence of cytokeratin.

The enteric nervous system (ENS) is the intrinsic nervous system of the gastrointestinal tract. It contains complete reflex circuits that detect the physiological condition of the gastrointestinal tract, integrate information about the state of the gastrointestinal tract, and provide outputs to control gut movement, fluid exchange between the gut and its lumen, and local blood flow. The ENS works in concert with the central nervous system (CNS) to control the digestive system in the context of local and whole body physiological demands.

The ENS originates from neural crest cells. These cells proliferate and differentiate into neurons and glial cells, and form two concentric plexuses of ganglion cells localized in the muscle layers of the gut wall (Furness, J. B. (2006). The organisation of the autonomic nervous system: peripheral connections. Auton. Neurosci. 130, 1-5. doi:10.1016/j.autneu.2006.05.003). In some embodiments, the tissue explant described herein comprises neural cells. In some embodiments, neural cells are identified by the presence of nestin. Nestin is an intermediate filament protein that is a known neural stem/progenitor cell marker.

b. Colon

In some embodiments, the tissue explant is derived from the colon. The colon is a part of the digestive system that functions in the absorption of water, electrolytes, and nutrients that remain after passing through the small intestine, and also in the compaction of feces. The lining of the colon, and its innermost layer, is the mucosa. The tunica serosa is the outermost covering of the digestive tube. It is comprised of an irregular dense connective tissue surrounded by a mesothelium, a type of squamous epithelium. Underneath the tunica serosa is the muscularis externa, comprising two muscle layers of an inner circular and outer longitudinal muscle. Between the layers are nervous plexus (Auberbach's myenteric). A fibroelastic connective tissue is found at the next level. Called the submucosa, it contains submucosal (Meissner) nervous plexuses, pre- and post-ganglionic parasympathetic fibers, and nonmyelinated preganglionic fibers from the vagus nerve. The innermost layer and lining of the colon is the mucosa. It comprises of an epithelium, a lamina propria, and muscularis mucosae. The epithelium is a simple columnar absorptive epithelium. The lamina propria is a loose connective tissue beneath the epithelium, and the muscularis mucosae is a thin smooth muscle cell layer surrounding the mucosa. The mucosa contains glands or crypts. The crypts comprise goblet cells and regenerative cells or enterocytes. The lamina propria (LP) fills the spaces between the crypts. The crypts are filled with large numbers of goblet cells that secrete mucus to lubricate ejection of the feces.

In some embodiments, the tissue explant described herein retains the in vivo architecture of the colon tissue from which it is derived. For example, in some embodiments, the issue explant comprises the epithelium and lamina propria of the colon. In some embodiments the tissue explant comprises the epithelium, lamina propria and muscularis mucosae of the colon. In some embodiments, the tissue explant further comprises the inner circular muscle from the muscularis externa of the colon. In some embodiments, the tissue explant comprises the inner circular and longitudinal muscle of the muscularis externa. In some embodiments, the tissue explant further comprises the submucosa of the colon. In some embodiments, the tissue explant further comprises intact crypts found in the colon. In some embodiments, the tissue explant derived from the colon comprises a mucus layer. In some embodiments, the tissue explant derived from the colon comprises a mucus layer and bowel content present on the apical side of the colon. In some embodiments, a tissue explant derived from the colon comprising a mucus layer and bowel content present on the apical side of the colon is useful for microbiome studies.

c. Stomach

In some embodiments, the tissue explant is derived from stomach, or gastric, tissue. The stomach is a muscular, hollow, dilated part of the alimentary canal. It comprises a mucosal layer comprising mucosal epithelium and lamina propria; which is surrounded by a submucosal layer comprising loose connective tissue; which is surrounded by a muscularis layer comprising several thick layers of muscle. The mucosal epithelium is comprised of four major types of secretory epithelial cells: mucous cells, which secrete an alkaline mucus that protects the epithelium against shear stress and acid; parietal cells, which secrete hydrochloric acid; chief cells (also called “peptic cells”) which secrete the zymogen pepsinogen; and G cells, which secrete the hormone gastrin. Cells within the mucosal epithelium can be identified by methods known to those of skill in the art. The epithelium is folded into thousands of tiny pits, called gastric pits, at the base of which are gastric glands; the mucous cells reside at the neck of the pits, while the chief cells and parietal cells residue at the base of the pits, in the glandular zone. Other markers of terminal gastric epithelial differentiation include H+/K+ atpase and mucin (MUC5A).

Stomach tissue also comprises a stomach-specific stem cell, a villin⁺Lgr⁵⁺ cell which is able to give rise to all gastric cell lineages. Current molecular markers for gastric progenitor cells and gastric cancer stem cells are described in J. Gastroenterol. 2011 July; 46(7):855-65, the disclosure of which is incorporated herein by reference.

In some embodiments, the tissue explant described herein retains the in vivo architecture of the stomach tissue from which it is derived. For example, in some embodiments, the tissue explant comprises the mucosal epithelium and lamina propria from the stomach. In some embodiments, the tissue explant further comprises the muscularis layer from the stomach. In some embodiments, the tissue explant derived from the stomach comprises mucous cells, parietal cells, chief cells, Gi cells, or combinations thereof. In some embodiments, the tissue explant derived from the stomach comprises villin+Lgr5+ stem cells.

d. Esophagus

In some embodiments, the tissue explant is derived from the esophagus. The esophagus is a muscular tube connecting the throat (pharynx) with the stomach. The esophagus is about 8 inches long and lined with mucosa. The upper esophageal sphincter (UIS) is a bundle of muscles at the top of the esophagus which is under conscious control. The lower esophageal sphincter (LES) is a bundle of muscles at the low end of the esophagus, where it meets the stomach, and is not under voluntary control. When closed, the LES prevents acid and stomach contents from traveling backwards.

The esophagus consists of mucosa, submucosa, layers of muscle fibers between layers of fibrous tissue, and an outer layer of connective tissue (serosa). The mucosa (innermost layer) is a stratified squamous epithelium of approximately three layers of squamous cells, which contrasts the single layer of columnar cells of the stomach. At the base of the mucosa lies the muscularis mucosa. The epithelial layer, connective tissue and muscularis mucosa comprise the mucosa.

In some embodiments, the tissue explant described herein retains the in vivo architecture of the esophageal tissue from which it is derived. For example, in some embodiments, the tissue explant comprises the mucosa of the esophagus. In some embodiments, the tissue explant comprises the mucosa and muscularis mucosa of the esophagus. In some embodiments, the tissue explant derived from the esophagus further comprises the serosa.

e. Buccal and Lingual

In some embodiments, the tissue explant is derived from buccal tissue (oral mucosa; relating to the mouth or cheek). In some embodiments, the tissue explant is derived from lingual tissue (relating to the tongue).

Buccal tissue consists of two layers, the surface stratified squamous epithelium and the deeper lamina propria. The epithelium consists of the following four layers: stratum basale, stratum spinosum, stratum granulosum, and stratum corneum. Depending on the region of the mouth, the epithelium may be keratinized or nonkeratinized. Nonkeratinized squamous epithelium covers the soft palate, inner lips, inner cheeks and floor of the mouth. Keratinized squamous epithelium is present in the attached gingiva and hard palate.

In some embodiments, the tissue explant retains the in vivo architecture of the buccal tissue from which it is derived. For example, in some embodiments, the tissue explant comprises the surface stratified squamous epithelium of the buccal tissue. In some embodiments, the tissue explant comprises the stratum basale, stratum spinosum, stratum granulosum, stratum corneum, or combinations thereof. In some embodiments, the tissue explant comprises the surface stratified squamous epithelium and the lamina propria of the buccal tissue. In some embodiments, the tissue explant derived from the buccal tissue comprises keratinized epithelium. In some embodiments, the tissue explant derived from the buccal tissue comprises nonkeratinized epithelium.

The tongue is a muscular organ in the mouth covered in mucosa. It is a mass of interlacing skeletal muscle, connective tissue with some mucous and serous glands, and pockets of adipose tissue. The tongue is anchored to the mouth via webs of tough tissue and mucosa. The tether holding down the front of the tongue is called the frenum. In the back of the mouth, the tongue is anchored into the hyoid bone. The tongue consists of lingual papillae, which are the small structure on the tipper surface of the tongue. Four types of papillae are found on the tongue: circumvallate papillae, fungiform papillae, filiform papillae and foliate papillae. All except the filiform papillae are associated with taste buds.

In some embodiments, the tissue explant described herein retains the in vivo architecture of the lingual tissue from which it is derived. For example, in some embodiments, the tissue explant comprises the connective tissue of the lingual tissue. In some embodiments, the tissue explant comprises mucous and serous glands present in the lingual tissue. In some embodiments, the tissue explant derived from the lingual tissue comprises intact lingual papillae. In some embodiments, the tissue explant derived from the lingual tissue comprises circumvallate papillae, fungiform papillae, filiform papillae, foliate papillae, or combinations thereof.

f. Rectum

In some embodiments, the tissue explant is derived from the rectum. The rectum is the final portion of the sigmoid colon and connects the sigmoid colon to the anal canal. The rectum is about 12 to 16 cm in length and has a histological structure similar to the rest of the large intestine. The rectum comprises mucosa, a submucosa, and muscularis propria. Epithelial cells in the mucosa include enterocytes and goblet cells.

In some embodiments, the tissue explant described herein retains the in vivo architecture of the rectal tissue from which it is derived. For example, in some embodiments, the tissue explant comprises the connective tissue of the rectum. In some embodiments, the tissue explant comprises the mucosa of the rectum. In some embodiments, the tissue explant comprises one or more of the mucosa, the submucosa, and the muscularis propria.

ii. Liver Tissue

In some embodiments, the tissue explant is derived from liver tissue. Tissue explants derived from liver tissue have been described in the art (see e.g., Othman A. et al. (2020) Archives of Toxicology 94:2889-91; WO2014197622 (Qu H. et al); WO2006041414 (Yu H & Khong Y M)). The liver is comprised of several lobes separated by surrounding serosa. Hexagonal like lobules comprising hepatocytes make up the structural units of the liver. Between the lobule structures are connective tissue and portal triads. These portal triads are composed of branches of hepatic arteries (i.e. endothelial cells), veins (i.e. endothelial cells), and bile ducts (i.e. epithelial cells). In some embodiments, the tissue explant described herein retains the in vivo architecture of the liver tissue from which it is derived. For example, in some embodiments, the tissue explant comprises the connective tissue of the liver. In some embodiments, the tissue explant comprises the lobules of the liver. In some embodiments, the tissue explant comprises portal triads of the liver. In some embodiments, the tissue explant comprises serosa of the liver. In some embodiments, the tissue explant comprises one or more of the connective tissue, lobules, portal triads and serosa of the liver. In some embodiments, the tissue explant comprises hepatocytes. In some embodiments, the tissue explant comprises hepatocytes and endothelial or epithelial cells, or hepatocytes, endothelial cells and epithelial cells.

iii. Heart Tissue

In some embodiments, the tissue explant is derived from heart tissue. Tissue explants derived from liver tissue have been described in the art (see e.g., Fischer C. et al. (2019) Nature Communications 10-532; Watson S. et al. (2019) Cardiovascular Drugs and Therapy 33, 239-244). The heart is made of four chambers: the left/right atria and the left/right ventricles. In some embodiments, the tissue explant is derived from a ventricle. In some embodiments, the tissue explant is derived from an atrium. The heart has three distinct layers known as the epicardium (encases the heart), the endocardium (i.e. the luminal surface), and the myocardium. The epicardium is a connective tissue sheath that encases the heart. The endocardium lines the inner chambers of the heart and composed of endothelial cells. The myocardium comprises the muscle cells (i.e. cardiomyocytes) and is highly vascularized with endothelial cells. In some embodiments, the tissue explant described herein retains the in vivo architecture of the heart tissue from which it is derived. For example, in some embodiments, the tissue explant comprises the cardiomyocytes of the heart. In some embodiments, the tissue explant comprises one or more of the epicardium, the myocardium, and the endocardium of the heart.

iv. Kidney Tissue

In some embodiments, the tissue explant is derived from kidney tissue (i.e. renal tissue). Tissue explants derived from pancreatic tissue have been described in the art (see e.g. Winbanks et al. (2011) BioMed Research International Vol. 2011. Article ID 212819; Poosti F et al. (2015) Disease Models and Mechanisms 8:1227-1236). The kidneys are surrounded by three layers of tissue. The outermost layer is a connective tissue layer called the renal fascia followed by the perineal fat layer and finally the renal capsule. The internal region of the kidney comprises three regions known as the outer cortex, medulla, and renal pelvis. The functional unit of the kidney is known as a nephron which is composed of cuboidal epithelial cells. The nephrons are found in the medulla and cortex of the kidney. The barrier of the kidney filtration system to that of the blood is comprised in part by glomerular endothelial cells which are located near nephrons. In some embodiments, the tissue explant described herein retains the in vivo architecture of the renal tissue from which it is derived. For example, in some embodiments, the tissue explant comprises the renal fascia of the kidney. In some embodiments, the tissue explant comprises the perineal fat of the kidney. In some embodiments, the tissue explant comprises the renal capsule of the kidney. In some embodiments, the tissue explant comprises the medulla of the kidney. In some embodiments, the tissue explant comprises the outer cortex of the kidney. In some embodiments, the tissue explant comprises the renal pelvis of the kidney. In some embodiments, the tissue explant comprises one or more of the perineal fat, the renal capsule, the renal fascia, the renal pelvis, the cortex, and the medulla of the kidney. In some embodiments, the tissue explant comprises at least one nephron. In some embodiments, the tissue explant comprises at least one nephron and a population of glomerular endothelial cells.

v. Splenic Tissue

In some embodiments, the tissue explant is derived from splenic tissue. The spleen is a lymphoid organ covered by a layer of visceral peritoneum. The reticular connective tissue of the spleen provides structure and support for the two distinct compartments of the tissue. The compartments include the red pulp which comprises macrophages, plasmocytes, and blood cells, and the white pulp which comprises T lymphocytes and B lymphocytes. In some embodiments, the tissue explant described herein retains the in vivo architecture of the splenic tissue from which it is derived. For example, in some embodiments, the tissue explant comprises the reticular connective tissue of the spleen. In some embodiments, the tissue explant comprises the red pulp (i.e., cords if Billroth and splenic sinusoids) of the spleen. In some embodiments, the tissue explant comprises the white pulp (i.e. the periarterial lymphoid sheath, lymphoid follicles, and the marginal zone) of the spleen. In some embodiments, the tissue explant comprises one or more of the reticular connective tissue, the red pulp, and the white pulp of the spleen.

vi. Pancreatic Tissue

In some embodiments, the tissue explant is derived from the pancreatic tissue. Tissue explants derived from pancreatic tissue have been described in the art (see e.g., Esni F. et al. Methods in Molecular Medicin, Pancreatic Cancer: Methods and Protocols Vol. 103, 259-271; Marciniak A. et al. (2013) PloS ONE. 8(11): e78706). The pancreas is surrounded by a layer of connective tissue. The parenchyma of the pancreas includes cells with endocrine and exocrine function. The exocrine or secretory units of the pancreas comprise epithelial cells known as Acini. The secretions from Acini leave the pancreas through a system of ducts made of epithelial cells. The endocrine units of the pancreas comprise Islets of Langerhans which comprise alpha cells, beta cells, delta cells, epsilon cells, and pancreatic polypeptide (PP) cells. In some embodiments, the tissue explant described herein retains the in vivo architecture of the pancreatic tissue from which it is derived. For example, in some embodiments, the tissue explant comprises the connective tissue of the pancreas. In some embodiments, the tissue explant comprises the Islets of Langerhans of the pancreas. In some embodiments, the tissue explant comprises Acini of the pancreas. In some embodiments, the tissue explant comprises one or more of the connective tissue, the Islets of Langerhans, and the Acini of the pancreas.

vii. Skin Tissue

In some embodiments, the tissue explant is derived from skin tissue. Tissue explants derived from skin tissue have been described in the art (see e.g., US20200400652A1; US20110045477A1). The skin is composed of three layers, the epidermis, the dermis, and the hypodermis. The epidermis is the outer layer of skin comprised of keratinized epithelium, keratinocytes, and melanocytes. The dermis underlies the epidermis and is comprised of connective tissue, hair follicles, sweat glands, and epithelial cells. In some embodiments, the tissue explant comprises the epidermis of the skin. In some embodiments, the tissue explant comprises the dermis of the skin. In some embodiments, the tissue explant comprises connective tissue of the skin. In some embodiments, the tissue explant comprises keratinocytes. In some embodiments, the tissue explant comprises melanocytes of the skin. In some embodiments, the tissue explant comprises one or more of keratinized epithelium, keratinocytes, melanocytes, connective tissue, hair follicles, sweat glands, and epithelial cells of the skin.

### C. Method for Obtaining Tissue Explant

The tissue explant described herein provides for culture, maintenance of in vivo architecture and recapitulation of tissue function, for example, long term or prolonged culture, maintenance of in vivo architecture and recapitulation of tissue function and use in methods described herein. The tissue explants described herein are useful for analysis of the tissue of interest (e.g., small intestine) cancer research, and high-throughput screening assays.

In some embodiments, the tissue explant described herein is derived from a source tissue. In some embodiments, the tissue explant described herein is derived from either a human or a large, non-human mammal. In some embodiments, the large, non-human mammal, includes ungulates (i.e., hoofed mammals such as pigs, cows, goats, sheep, horses, donkeys, deer, antelopes and the like) and more generally, livestock (i.e., mammals raised for agricultural purposes such as pigs, cows, goats, sheep, horses, rabbits, and the link, and/or as beasts of burden such as donkeys, horses, elephants, camels, llamas, and the like). In some embodiments, the large, non-human mammal is a pig.

In some embodiments, the source tissue comprises an architecture and the tissue explant comprises said architecture. In some embodiments, tissue architecture is determined by the cellular and extracellular components. Cell-cell interactions, and cell-extracellular matrix interactions collectively define a tissue's architecture. In some embodiments, the tissue explant has the same cell-cell interactions as the source tissue. In some embodiments, the tissue explant has the same cell-extracellular matrix interactions as the source tissue.

In some embodiments, the tissue of interest (e.g., small intestine) is obtained surgically. In some embodiments, the tissue of interest (e.g., small intestine) is obtained surgically post-exsanguination (i.e., draining of blood). In some embodiments, the tissue explant obtained is the length and width of the substrate of interest. In some embodiments, the tissue explant obtained is the length and width of a standard 6, 12, 24, 48, 96, 384, 1536 or 3456 well plate. In some embodiments, the tissue explant obtained is the length and half the width of a standard 6, 12, 24, 48, 96, 384, 1536 or 3456 well plate. In some embodiments, the tissue explant is about 127.8 mm in length and about 42.75 mm in width. In some embodiments, the tissue explant is about 127.8 mm in length and 85.5 mm in width.

In some embodiments, the age of the animal can have an effect on the maintenance and function of the tissue explant. In some embodiments, the animal is between 3 weeks and 12 weeks of age. In some embodiments the animal is 3 weeks of age. In some embodiments the animal is 12 weeks of age. In some embodiments the animal is 1, 2, 3, 4, 5, 6, 7, 8, 9, 10, 11, 12, 13, 14 or 15 weeks of age. In some embodiments, the animal is 1, 2, 3, 4, 5, 6 or 7 months old. In some embodiments, fetal tissue is utilized.

In some embodiments, the tissue explant is immersed in a series of saline solutions after dissection. In some embodiments, the tissue explant is immersed in 70% ethanol after dissection, followed by washing with saline solutions. In some embodiments, the saline solutions are supplemented with an antibiotic solution. In some embodiments, the saline solutions are supplemented with an antimycotic solution. In some embodiments, the saline solutions are supplemented with an antibiotic and antimycotic solution. Antibiotic and antimycotic solutions are known by those of skill in the art. For example, Gibco® Antibiotic-Antimycotic solution is useful in the methods described herein. In some embodiments, the antibiotic and/or antimycotic solution comprises penicillin, streptomycin, Gibco® amphotericin B, or combinations thereof.

In some embodiments, the tissue explant is immersed in a known preservation solution. Examples of preservation solutions include, but are not limited to, Krebs-Henseleit solution, UW solution, St. Thomas II solution, Collins solution, and Stanford solution (See, for example, U.S. Pat. Nos. 4,798,824 and 4,938,961; Southard and Belzer, Ann. Rev. Med. 46:235-247 (1995); and Donnelly and Djuric, Am. J. Hosp. Pharm. 48:2444-2460 (1991)). The solution may contain one or more of sodium, potassium, calcium, magnesium, glutamate, arginine, adenosine, mannitol, allopurinol, glutathione, raffinose, and lactobionic acid. In some embodiments, the solution is maintained at physiological pH of about 7.2-7.4.

In some embodiments, the tissue is kept on ice before dissection. Therefore, in some embodiments the solutions are 4° C. before being used.

The tissue explant is subsequently mounted on the substrate of interest (e.g., multi-well plate) and cultured in culture media at 37° C. in an airtight container. In some embodiments, the culture media is free of serum. In some embodiments, the culture media comprises serum.

In some embodiments, the culture media does not contain exogenous growth factors (e.g., Wnt3a). In some embodiments, the tissue explant does not require exogenous growth factors due to the presence of the stromal layer. In some embodiments, the culture media is Dulbecco's Modified Eagle Medium (DMEM) or Advanced DMEM/F-12. In some embodiments, the culture media includes fetal bovine serum (FBS). In some embodiments, the culture media include EGF Recombinant Human Protein. In some embodiments, the presence of FBS and/or EGF does not affect the viability of the tissue explant.

In some embodiments, the tissue explant is derived from the gastrointestinal tract of a human or large, non-human mammal. The gastrointestinal tract comprises the mouth, esophagus, stomach and or rumen, intestines (small and large), cecum (plural ceca), fermentation sacs, and the anus. In some embodiments, the tissue explant is derived from the intestine. In some embodiments, the tissue explant is derived from the small intestine.

The roughly 8 meters of intestine in the adult human plays numerous roles in physiologic homeostasis including absorptive, secretory and immune functions. Commensurate with these essential roles, diseases of the intestine are a considerable source of human morbidity and mortality. Indeed, numerous pathologic conditions including cancer, inflammatory bowel diseases, mesenteric ischemia, congenital syndromes and trauma, with or without concomitant intestinal resection, result in “short-gut” syndromes resulting in severe deficiencies of physiologic intestinal function and effective intestinal failure.

The intestine is an organ with tremendous regenerative potential, whereby stem cells resident in proliferative crypt regions give rise to progenitors capable of multilineage differentiation. The intestinal stem cells (ISCs) are able to repopulate epithelium of the entire 8-meter length of the adult human intestine every 5-7 days, helping to maintain the integrity of the mucosal barrier and effecting tissue repair upon injury. It has been postulated that the ISC niche has complex architectural requirements whereby myofibroblasts enveloping the proliferative crypt provide essential signals to crypt stem and/or progenitor cells.

The small intestine has three distinct regions, the duodenum, jejunum and ileum. The duodenum is connected to the distal end of the stomach and receives bile and pancreatic juice through the pancreatic duct. The jejunum and ileum primarily absorb nutrients and water more so than the breaking down of food.

In some embodiments, the tissue explant is derived from the jejunum of the small intestine. In some embodiments, the tissue explant is derived from the ileum of the small intestine. In some embodiments, the tissue explant is derived from the duodenum of the small intestine. In some embodiments, the tissue explant is derived from the stomach. In some embodiments, the tissue explant is derived from the esophagus. In some embodiments, the tissue explant is derived from buccal tissue. In some embodiments, the tissue explant is derived from lingual tissue. In some embodiments, the tissue explant is derived from the colon. In some embodiments, the tissue explant is derived from the heart. In some embodiments, the tissue explant is derived from the liver. In some embodiments, the tissue explant is derived from the kidney. In some embodiments, the tissue is derived from the pancreas. In some embodiments, the tissue explant is derived from the spleen.

### D. Modification of Tissue Explant

The tissue explant described herein may be experimentally modified. In some embodiments, the tissue explant is modified prior, or during the culture period. In some embodiments, the tissue explant is modified by exposure to viral or bacterial pathogens. In some embodiments, the tissue explant is modified by altering patterns of gene expression (e.g., by providing reprogramming factors). In some embodiments, the tissue explant is modified through genetic modification. In some embodiments, genetic modification includes, but is not limited to knocking down genes with, for example, interfering RNAs (shRNA, siRNA), and stable genetic modification with, for example, CRISPR/Cas9. The experimentally modified tissue explant is useful for investigation of the effects of drug transporters or drug metabolizing enzymes; the effects of therapeutics agents; for tumor therapy, for effects on differentiation, and the like.

In some embodiments, expression of drug transporters and/or drug metabolizing enzymes is modified. In some embodiments, expression of drug transporters and/or drug metabolizing enzymes is knocked down. In some embodiments, expression of at least one drug transporter is modified. In some embodiments, expression of at least one drug transporter is knocked down. In some embodiments, expression of at least one drug metabolizing enzyme is modified. In some embodiments, expression of at least one drug metabolizing enzyme is knocked down.

In some embodiments, the tissue explant is modified to generate a pathological condition. Examples of pathological conditions include, but are not limited to, inflammatory bowel diseases (IBD), colon cancer, mesenteric ischemia, congenital syndromes and trauma, which can produce functional loss or mandate physical resection of large sections of intestine extensive enough to compromise organ physiology. The ability to maintain tissue explants in culture is valuable for development of therapies for treating intestinal diseases and trauma induced intestinal failure.

Methods for modifying cells or tissue are known to one of skill in the art. For example, introduction of an expression vector encoding a polypeptide can be used to express the encoded product in cells lacking the sequence, or to over-express the product. Various promoters can be used that are constitutive or subject to external regulation, where in the latter situation, one can turn on or off the transcription of a gene. These coding sequences may include full-length cDNA or genomic clones, fragments derived therefrom, or chimeras that combine a naturally occurring sequence with functional or structural domains of other coding sequences. Alternatively, the introduced sequence may encode an anti-sense sequence; be an anti-sense oligonucleotide; siRNA or a shRNA, encode a dominant negative mutation, or dominant or constitutively active mutations of native sequences; altered regulatory sequences, etc. Instead of being expressed from a vector transfected or transduced into the tissue explant, the oligonucleotides, siRNA or shRNA can be directly transfected or transduced into the tissue explant.

In addition to sequences derived from the host cell species, other sequences of interest include, for example, genetic sequences of pathogens, for example coding regions of viral, bacterial and protozoan genes, particularly where the genes affect the function of human or other host cells. Sequences from other species may also be introduced, where there may or may not be a corresponding homologous sequence.

A large number of public resources are available as a source of genetic sequences, e.g. for human, other mammalian, and human pathogen sequences. A substantial portion of the human genome is sequenced, and can be accessed through public databases such as Genbank. Resources include the uni-gene set, as well as genomic sequences. For example, see Dunham et al. (1999) Nature 402, 489-495; or Deloukas et al. (1998) Science 282, 744-746.

cDNA clones corresponding to many human gene sequences are available from the IMAGE consortium. The international IMAGE Consortium laboratories develop and array cDNA clones for worldwide use. The clones are commercially available, for example from Genome Systems, Inc., St. Louis, Mo. Methods for cloning sequences by PCR based on DNA sequence information are also known in the art.

Methods that are well known to those skilled in the art can be used to construct expression vectors containing coding sequences and appropriate transcriptional and translational control signals for increased expression of an exogenous gene introduced into a cell. These methods include, for example, in vitro recombinant DNA techniques, synthetic techniques, and in vivo genetic recombination. Alternatively, RNA capable of encoding gene product sequences may be chemically synthesized using, for example, synthesizers. See, for example, the techniques described in “Oligonucleotide Synthesis”, 1984, Gait, M. J. ed., IRL. Press, Oxford.

A variety of host-expression vector systems may be utilized to express a genetic coding sequence. Expression constructs may contain promoters derived from the genome of mammalian cells, e.g., metallothionein promoter, elongation factor promoter, actin promoter, etc., from mammalian viruses, e.g., the adenovirus late promoter; the vaccinia virus 7.5K promoter, SV40 late promoter, cytomegalovirus, etc.

In mammalian host cells, a number of viral-based expression systems may be utilized, e.g. retrovirus, lentivirus, adenovirus, herpesvirus, and the like. In cases where an adenovirus is used as an expression vector, the coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the gene product in infected hosts (see Logan & Shenk, 1984, Proc. Natl Acad. Sci. USA 81:3655-3659. Specific initiation signals may also be required for efficient translation of inserted gene product coding sequences. These signals include the ATG initiation codon and adjacent sequences. Standard systems for generating adenoviral vectors for expression on inserted sequences are available from commercial sources, for example the Adeno-X™ expression system from Clontech (Clontechniques, January 2000, p. 10-12).

In cases where an entire gene, including its own initiation codon and adjacent sequences, is inserted into the appropriate expression vector, no additional translational control signals may be needed. However, in cases where only a portion of the gene coding sequence is inserted, exogenous translational control signals, including, perhaps, the ATG initiation codon must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., 1987, Methods in Enzymol. 153:516-544).

In some embodiments, methods are used that achieve a high efficiency of transfection, and therefore circumvent the need for using selectable markers. These may include physical modes of delivery including microneedles, microjets, iontophoresis, and ultrasound mediated siRNA delivery.

### Exemplary Tissue Compositions

In some embodiments, the tissue explant is derived from swine gastrointestinal tissue. In some embodiments, the tissue explant is derived from swine large intestine tissue. In some embodiments, the tissue explant is derived from swine small intestine tissue. In some embodiments, the tissue graft is an organoid. In some embodiments, the organoid is derived from swine tissue. In some embodiments, the organoid is derived from swine colon tissue. In some embodiments, the tissue explant is derived from swine gastrointestinal tissue and the tissue graft is an organoid derived from swine colon tissue. In some embodiments, the tissue explant is derived from swine large intestine tissue and the tissue graft is an organoid derived from swine colon tissue. some embodiments, the tissue explant is derived from swine small intestine tissue and the tissue graft is an organoid derived from swine colon tissue.

In some embodiments, cells from gastrointestinal tissue (e.g., swine gastrointestinal tissue) are used to form an organoid. In some embodiments, cells from healthy gastrointestinal tissue (e.g., swine gastrointestinal tissue) are used to form an organoid. In some embodiments, cells from cancerous gastrointestinal tissue (e.g., swine gastrointestinal tissue) are used to form an organoid. In some embodiments, cells from healthy gastrointestinal tissue (e.g., swine gastrointestinal tissue) are used to form a tumorigenic organoid. In some embodiments, cells from healthy gastrointestinal tissue (e.g., swine gastrointestinal tissue) are gene edited. In some embodiments, cells from healthy gastrointestinal tissue (e.g., swine gastrointestinal tissue) are gene edited to form a tumorigenic organoid. In some embodiments, cells from healthy gastrointestinal tissue (e.g., swine gastrointestinal tissue) are gene edited to knock-down expression of a gene to form a tumorigenic organoid. In some embodiments, cells from healthy gastrointestinal tissue (e.g., swine gastrointestinal tissue) are gene edited to knock-down expression of p53, APC, SMAD4 or any combination thereof to form a tumorigenic organoid. In some embodiments, cells from colon tissue (e.g., swine colon tissue) are used to form an organoid. In some embodiments, cells from healthy colon tissue (e.g., swine colon tissue) are used to form an organoid. In some embodiments, cells from cancerous colon tissue (e.g., swine colon tissue) are used to form an organoid. In some embodiments, cells from healthy colon tissue (e.g., swine colon tissue) are used to form a tumorigenic organoid. In some embodiments, cells from healthy colon tissue (e.g., swine colon tissue) are gene edited. In some embodiments, cells from healthy colon tissue (e.g., swine colon tissue) are gene edited to form a tumorigenic organoid. In some embodiments, cells from healthy swine colon tissue are gene edited to create a colorectal cancer organoid.

In some embodiments, cells from healthy colon tissue (e.g., swine colon tissue) are gene edited to knock-down expression of a gene to form a tumorigenic organoid. In some embodiments, cells from healthy colon tissue (e.g., swine colon tissue) are gene edited to knock-down expression of p53, APC, SMAD4 or any combination thereof to form a tumorigenic organoid. In some embodiments, cells from healthy swine colon tissue are gene edited to knock-down expression of p53, APC and SMAD4 to form a tumorigenic organoid.

In some embodiments, the organoids are cultured for more than one passage before placement within the tissue explant. In some embodiments, the organoids are cultured for two passages before placement within the tissue explant. In some embodiments, the organoids are cultured in two passages over 14 days of culture before placement within the tissue explant. In some embodiments, an organoid is placed within the tissue explant. In some embodiments, tissue explants described herein comprise more than one layer of cells. In some embodiments, an organoid is placed between two layers of a tissue explant. In some embodiments, an organoid is placed between two layers of cells of the explant. In some embodiments, an organoid is placed between a layer of cells and a connective tissue layer of the explant. In some embodiments, an organoid is placed within a layer of a tissue explant. In some embodiments, the tissue explant comprises a mucosal layer and an organoid is placed within the mucosal layer. In some embodiments, the organoid is placed under the mucosa layer. In some embodiments, the organoid is placed above the submucosal layer. In some embodiments, the organoid is placed between two layers of the tissue explant (e.g. between the mucosa and the submucosa layers). In some embodiments, the tissue explant comprises an epithelial layer and an organoid is located subepithelial.

In some embodiments, an organoid placed within the tissue explant develops tumors. In some embodiments, an organoid is excised from the tissue explant for morphological and/or protein analysis. APC targets β-catenin for degradation therefore, loss of APC and an increase in β-catenin is an indicator of cancer development. Similar to β-catenin, cytokeratin 20 is often used as a marker for cancer development as an increase in expression indicates increased cell differentiation and cancer formation. In some embodiments, an organoid placed within the tissue explant expresses β-catenin. In some embodiments, an organoid placed within the tissue explant expresses keratin 20 (KRT20).

### Methods of Making the Tissue Compositions of the Disclosure

Also provided herein are methods for making the tissue composition of the disclosure. In some embodiments, a graft is placed within a tissue explant described herein. In some embodiments, the tissue explant is cultured for 1, 2, 3, 4, 5, 6, 7, 8, 9, 10, 11, 12, 13, or 14 days prior to placement of a graft within the explant.

In some embodiments, the graft is injected into the tissue explant. In some embodiments, the graft is placed within the tissue explant. In some embodiments, the graft is in a solution of cell culture medium. In some embodiments, the graft is placed within a layer of the explant. In some embodiments, the graft is placed between two layers of the explant. In some embodiments, the graft is placed between two cell layers of the explant. In some embodiments, the graft is placed between a cell layer and a connective tissue layer of the explant. In some embodiments, the graft is placed within any one of the mucosa, the submucosa, the muscularis externa, or serosa of the intestinal tissue explant. In some embodiments, the graft is placed under the mucosa layer. In some embodiments, the graft is placed above the submucosal layer. In some embodiments, the graft is placed between two layers of the tissue explant (e.g. between the mucosa and the submucosa layers). In some embodiments, the graft is placed within the mucosal layer of the tissue explant. In some embodiments, the graft is placed between the mucosal layer and the submucosa of the tissue explant.

In some embodiments, the graft is an organoid and the organoid is first isolated from its culture matrix (e.g. Matrigel) prior to placement within the tissue explant. In some embodiments, the organoid is cultured for three days before placement into the tissue explant. In some embodiments, the organoid is placed within the mucosal layer of the tissue explant.

In some embodiments, the graft is placed within a location of the tissue explant that corresponds to a well of the substrate in contact with the tissue explant. In some embodiments, organoids are placed within a location of the tissue explant that corresponds to a well of the substrate in contact with the tissue explant. As described herein, in some embodiments the substrate comprises a single-well or a plurality of wells. The presence of multiple wells enables the tissue explant to be assayed under multiple conditions. In some embodiments, organoids are placed within the tissue explant in a location corresponding to one or more wells of a substrate described herein. In some embodiments, more than one tissue graft (e.g., organoid) is placed within the tissue explant in a location corresponding to one or more wells of a substrate. For example, in some embodiments, 100, 250, 500, 1000, 1500, 2000, 2500, or 3000 organoids are placed within the tissue explant in a location corresponding to one well of a substrate. In some embodiments, tissue grafts (e.g., organoids) are placed at one or more locations within the tissue explant corresponding to one or more wells of the substrate. In some embodiments, 2000 organoids are placed within the mucosal layer the tissue explant in a location corresponding to one well of a substrate described herein. In some embodiments, tissue grafts (e.g., organoids) are placed within the tissue explant in a location corresponding to each well of a substrate described herein.

In some embodiments, the graft is derived from a cells. In some embodiments, about 500, about 1000, about 1500, about 2000, about 2500, about 3000, about 3500, or about 4000 cells are placed within the tissue explant. In some embodiments, about 4000 cells are placed within the tissue explant.

In some embodiments, the graft is derived from a cell line. In some embodiments, about 500, about 1000, about 1500, about 2000, about 2500, about 3000, about 3500, or about 4000 cells from a cell line are placed within the tissue explant. In some embodiments, about 4000 cells from a cell line are placed within the tissue explant.

In some embodiments, the tissue graft is placed within the tissue explant by injection. In some embodiments, the tissue graft is in a cell culture medium and is placed within the tissue explant by injection. In some embodiments, organoids are placed within the tissue explant by injection. In some embodiments, organoids are in a cell culture medium and placed within the tissue explant by injection. In some embodiments, tissue grafts (e.g., organoids) are injected into a tissue explant using a syringe needle. In some embodiments, the gauge of the syringe need is determined based on the size of the tissue graft (e.g., organoid). In some embodiments, tissue grafts (e.g., organoids) are injected using a 19G-25G syringe needle. In some embodiments, tissue grafts (e.g., organoids) are injected using a 19G, a 20G, a 21G, a 22G, a 23G, or a 25G syringe needle. In some embodiments, tissue grafts (e.g., organoids) are injected using a 23G syringe needle.

In some embodiments, tissue grafts (e.g., organoids) are cultured in the tissue explants for a sufficient amount of time to form a tumor. In some embodiments, the tissue graft (e.g., organoid) forms a tumor 3, 4, 5, 6, 7, 8, 9, or 10 days after placement within the tissue construct. In some embodiments, the tissue grafts (e.g., organoids) form tumors with similar morphology (e.g. formation of crypt structures) to a gastrointestinal tumor. Gastrointestinal tumors have various morphological patterns which include glandular features, mucinous structures, crypt structures, and medullary or medullary-like patterns. In some embodiments, a colorectal cancer (CRC) organoid is placed in the tissue explant for a sufficient amount of time to form a CRC tumor. In some embodiments, the CRC organoid expresses β-catenin. In some embodiments, the CRC organoid expresses cytokeratin 20 (KRT20).

In some embodiments, the tissue composition (i.e. tissue explant and graft) culture is maintained in Advanced/F12 medium comprising 10% fetal bovine serum and 5% antibiotic-antimycotic solution. In some embodiments, the tissue composition culture is maintained in Advanced/F12 medium comprising 10% fetal bovine serum and 5% antibiotic-antimycotic solution for 1, 2, 3, 4, 5, or 6 days to allow formation of tumors from the tissue grafts (e.g., organoids). In some embodiments, the tissue composition is maintained in Advanced/F12 medium comprising 10% fetal bovine serum and 5% antibiotic-antimycotic solution for three days to allow formation of tumors from the tissue grafts (e.g., organoids). In some embodiments, the media is changed every 6, 8, 10, 12, 14, or 16 hours. In some embodiments, the culture media for the tissue composition cultures is changed every 12 hours.

### Substrates for the Composition

In some embodiments, the tissue explant comprising a graft as described herein is placed on a substrate. Various culture substrates can be used in the methods and systems of the disclosure. Such substrates include, but are not limited to, glass, polystyrene, polypropylene, stainless steel, silicon and the like. In some embodiments, the substrate is poly(methyl methacrylate). In some embodiments, the substrate is a polycarbonate, acrylic copolymer, polyurethane, aluminum, carbon or Teflon (polytetrafluoroethylene). The cell culture surface can be chosen from any number of rigid or elastic supports. For example, cell culture material can comprise glass or polymer microscope slides. In some embodiments, the substrate may be selected based upon a tissue's propensity to bind to the substrate. In some embodiments, the substrate may be selected based on the potential effect of the substrate on the tissue explant (e.g., electrical stimulation/resistivity, mechanical stimulation/stress).

The cell culture surface/substrate can be made of any material suitable for culturing mammalian cells. For example, the substrate can be a material that can be easily sterilized such as plastic or other artificial polymer material, so long as the material is biocompatible. In some embodiments, the substrate is any material that allows cells and/or tissue to adhere (or can be modified to allow cells and/or tissue to adhere or not adhere at select locations). Any number of materials can be used to form the substrate/surface, including but not limited to, polyamides; polyesters; polystyrene; polypropylene; polylacrylates; polyvinyl compounds (e.g., polyvinylchloride); polycarbonate; polytetrafluoroethylene (PTFE); nitrocellulose; cotton; polyglyolic acid (PGA); cellulose; dextran; gelatin; glass; fluoropolymers; fluorinated ethylene propylene; polyvinylidene; polydimethylsiloxane; and silicon substrates (such as fused silica, polysilicon, or single silicon crystals), and the like. Also, metals (e.g., gold, silver, titanium films) can be used.

In some embodiments, the substrate may be modified to promote cellular adhesion (e.g., coated with an adherence material). For example, a glass substrate may be treated with a protein (i.e., a peptide of at least two amino acids) such as collagen or fibronectin to assist cells of the tissue in adhering to the substrate. In some embodiments, a single protein is adhered to the substrate. In some embodiments, two or more proteins are adhered to the substrate. Proteins suitable for use in modifying the substrate to facilitate adhesion include proteins to which specific cell types adhere under cell culture conditions.

The type of adherence material(s) (e.g., ECM materials, sugars, proteoglycans, etc.) deposited on the substrate will be determined, in part, by the cell type or types in the tissue explant.

In some embodiments, the substrate does not require adherence material. Prior gastrointestinal culture systems utilizing primary cells require exogenous extracellular matrix. In some embodiments, the tissue explant described herein does not require exogenous extracellular matrix.

In some embodiments, the substrate is a singular well plate. In some embodiments, the substrate is a multi-well plate or assembly. In some embodiments, the substrate comprises microwells. In some embodiments, the substrate comprises 6, 12, 24, 48, 96, 384 or 1536 microwells. In some embodiments, the substrate comprises 96 microwells. In some embodiments, the substrate comprises 384 microwells. In some embodiments, the substrate comprises 1536 microwells. In some embodiments, each microwell is completely covered by the tissue explant described herein.

In some embodiments, the microwells are through holes. In some embodiments, the microwells are receiving chambers. In some embodiments, the tissue explant is placed between a first plate and a second plate, wherein the first plate comprises a plurality of through holes and the second plate comprises a plurality of receiving chambers.

In some embodiments, the tissue explant described herein is placed on an interface apparatus comprising a standard plate, a thin middle plate, and an upper load plate. The tissue explant is placed over the through holes of the middle plate and the upper load plate is then placed onto the tissue explant to compress it onto the middle plate and around the through holes, while mounted on the standard plate. In some embodiments, each plate comprises 6, 12, 24, 48, 96, 384 or 1536 microwells.

In some embodiments, the upper load plate comprises posts having a diameter from 3 mm to 5 mm. In some embodiments, the upper load plate comprises posts having a diameter from about 3 mm to about 5 mm. In some embodiments, the upper load plate comprises posts having a diameter of 4 mm. In some embodiments, the upper load plate comprises posts having a diameter of about 4 mm. In some embodiments, the tissue explant placed on the middle plate is slightly recessed into each well by forces from the upper plate. In some embodiments, the middle plate thickness is 1 mm or 2 mm. In some embodiments, the middle plate thickness is about 1 mm or about 2 mm. In some embodiments, the middle plate thickness is 1 mm. In some embodiments, the middle plate thickness is about 1 mm. In some embodiments, the diameter of posts of the middle plate is larger than the diameter of the upper load plate to ensure the tissue explant rests between the upper and middle plate. In some embodiments, the middle plate comprises posts having a diameter from 6.5 mm to 8 mm. In some embodiments, the middle plate comprises posts having a diameter from about 6.5 mm to about 8 mm. In some embodiments, the middle plate comprises posts having a diameter of 6 mm. In some embodiments, the middle plate comprises posts having a diameter of about 6 mm.

In some embodiments, the outer dimensions of any of the plates described herein are no bigger than that which would fit in a 140 mm, 145 mm, 150 mm, or 200 mm petri dish. In some embodiments, the outer dimensions of any of the plates described herein are no bigger than that which would fit in a 140 mm petri dish.

In some embodiments, the pressure applied to the tissue explant minimizes well-to-well leakage. In some embodiments, the pressure applied to the tissue explant is 20N, 15N, 10N, or 5N. In some embodiments, the pressure applied to the tissue explant is about 20N, about 15N, about 10N, or about 5N. In some embodiments, the pressure applied to the tissue explant is 5N. In some embodiments, the pressure applied to the tissue explant is about 5N.

### Methods of Use

In some embodiments, the tissue compositions described herein are used to determine response to single and combination therapies in cancer. In some embodiments, the results obtained from the methods described herein are used to develop machine learning prediction algorithms to predict new therapies.

### Predicting or Determining Drug Cytotoxicity

In some aspects of the disclosure, the tissue composition (i.e. the tissue explant and tissue graft) described herein is useful for predicting the cytotoxicity of a compound or composition of interest. In further aspects of the disclosure, the tissue composition described herein is useful for studying drug transport mechanisms.

Physiologically relevant models for studying gastrointestinal cancer and the efficacy of therapeutic compounds is limited. The intestinal tract of mouse models lacks physiological features that are important to human tumor development. Available in vitro models such as 3D cell culture lack the complex interaction between tumor tissue and the resident organ tissue. These available models fail to fully recapitulate the complex in vivo architecture and function of the gastrointestinal tract (e.g., small intestine). The tissue composition described herein provides significant advantages over the current model systems. For example, as discussed supra, the tissue explant described herein maintains the in vivo architecture of the gastrointestinal tract (e.g., small intestine) from which it was derived. In addition, the tissue composition comprises the components necessary for measuring cytotoxicity of therapeutic compounds. The tissue composition described herein can also be maintained in culture for long periods of time, unlike previously developed systems. Further, the tissue composition described herein does not require exogenous factors for maintenance in culture. Moreover, as discussed infra, the tissue composition described herein can be used for high-throughput screening. These characteristics highlight the improvements over prior model systems.

The tissue composition described herein provides a model system for testing and predicting drug cytotoxicity. Effective drug therapy relies on the interplay between the pharmacokinetics and pharmacodynamics (PK/PD) of the compound upon administration. During the initial stages of drug discovery, numerous studies are performed to assess the pharmacological effectiveness of new chemical entities (NCEs) to select a lead compound(s) that offers the greatest promise for therapeutic efficacy. The tissue explants described herein offer a unique tool for measuring cytotoxicity of candidate agents.

In some aspects of the disclosure, candidate drug formulations are screened for their cytotoxicity toward the tissue graft. The effect of a formulation is determined by adding the compound of interest in combination with a formulation to the tissue composition described herein, and measuring the cytotoxicity toward the tissue graft.

In some embodiments, candidate drug formulations are screened for their anti-tumorigenic effect. For example, in some embodiments, a method for measuring cytotoxicity comprises: (1) combination of drug+solvent to form a drug solution; (2) contacting the tissue composition with the drug solution; and (3) detection of cytotoxicity using known methods and methods described herein. Upon analysis, successful drug or drug combinations are identified.

In some embodiments, candidate agents are screened for their toxicity effect. The tissue explant is exposed to the candidate agent or vehicle, and its viability, maintenance in culture and architecture is assessed. In some embodiments, a toxic agent decreases tumor (i.e. tissue graft) viability. In some embodiments, a toxic agent decreases the time in which the tissue graft is maintained in culture. In some embodiments, a toxic agent modifies the architecture of the tissue graft.

The tissue composition described herein is capable of analyzing tumor/cancer toxicity with higher in vivo predictability compared to conventional in vitro assays. In some embodiments, the tissue composition is used as a screening platform to predict tumor/cancer toxicity and/or gastrointestinal side effects.

In another aspect of the disclosure, a method is provided for screening for agents for their effect on cells of different tissues, including processes of cancer initiation and treatment. Tissue compositions cultured by the methods described herein are exposed to candidate agents. Agents of interest include pharmaceutical agents, e.g. small molecules, antibodies, peptides, etc., and genetic agents, e.g. antisense, RNAi, expressible coding sequences, and the like, e.g. expressible coding sequences for candidate tumor suppressors, candidate oncogenes, and the like. In some embodiments, the effect on stem cells is determined. In other embodiments the effect of transformation or growth of tumor cells is determined, for example where agents may include, without limitation, chemotherapy, monoclonal antibodies or other protein-based agents, radiation/radiation sensitizers, cDNA, siRNA, shRNA, small molecules, and the like. Agents active on tissue-specific stem cells are detected by change in growth of the tissue explants and by the presence of multilineage differentiation markers indicative of the tissue-specific stem cell. In addition, active agents are detected by analyzing tissue explants for long-term reconstitutive activity. In some embodiments, the methods find use in identifying new agents for the treatment of disease. In some embodiments, the methods find use in determining effective delivery of already existing agents.

In some embodiments, the graft forms a tumor in the tissue explant before the tissue explant is contacted with any agent described herein. In some embodiments, any agent described herein is contacted with the tissue composition 1, 2, 3, 4, 5, 6, 7, 8, 9, 10, 11, 12, 13, or 14 days after the graft is placed within the tissue explant as described herein. In some embodiments, anti-cancer drugs are contacted with the tissue composition 1, 2, 3, 4, 5, 6, 7, 8, 9, 10, 11, 12, 13, or 14 days after the graft is placed within the tissue explant as described herein. In some embodiments, grafts are cultured in the tissue explants 1, 2, 3, 4, 5, 6, 7, 8, 9, 10, 11, 12, 13, or 14 days before contact with any agent described herein. In some embodiments, grafts are cultured in the tissue explants 1, 2, 3, 4, 5, 6, 7, 8, 9, 10, 11, 12, 13, or 14 days before contact with an anti-cancer drug described herein.

In some embodiments, the effect of a test compound is determined by conducting a first assay, contacting the tissue composition with a compound of interest, waiting for a sufficient period of time, conducting a second assay on the tissue composition, and comparing the results of the first assay and the second assay, to determine the effect of the compound.

Examples of assays for measuring drug cytotoxicity include, but are not limited to, effect on a tissue (e.g., genetic modifications, change in protein or gene expression, change in tissue histology/morphology), cell death, and hormone secretion. Examples of assays analyzing cytotoxicity include, but are not limited to, Live/Dead assays, alamarBlue®, and RayBio® Bioluminescence Cytotoxicity Assay Kit. In some embodiments, more than one assay is conducted simultaneously.

The agents are added in solution or readily soluble form, to the culture medium. The agents may be added in a flow-through system, as a stream, intermittent, continuous, or alternatively adding a bolus of the compound, singly or incrementally, to an otherwise static solution. In a flow-through system, two fluids are used, where one is a physiologically neutral solution, and the other is the same solution with the compound of interest added. The first fluid is passed over the cells, followed by the second. In a single solution method, a bolus of the test compound is added to the volume of media surrounding the tissue composition. In some embodiments, the compound of interest is injected directly into the tissue composition.

In some embodiments, the method for determining the effect of a candidate drug (e.g., cytotoxicity) on a tissue graft comprises:
(i) obtaining a tissue explant described herein
(ii) placing a tissue graft described herein within the tissue explant, therein making a tissue composition
(iii) contacting the tissue composition with the candidate drug;
(iv) measuring cell response within the tissue graft after treatment with the candidate drug for a period of time, thereby determining the effect of the candidate drug on cells of the tissue graft.

In some embodiments, the effect being measured is a cytotoxic effect on cells. In some embodiments, the effect being measured is proliferation of cells. In some embodiments, the effect being measured is cell cycle arrest. In some embodiments, the effect being measured is inhibitor of the epithelial to mesenchymal transition (EMT). Methods for measuring cytotoxicity, proliferation, cell cycle arrest, and EMT are known to those of skill in the art.

In some embodiments, the disclosure provides methods for determining the cytotoxic effect of a candidate drug on cancer cells. In some embodiments, the method for determining the cytotoxic effect of a candidate drug on cancer cells comprises:
(i) obtaining a gastrointestinal tract tissue explant comprising intestinal epithelium from a large mammalian gastrointestinal tract
(ii) placing a tumorigenic tissue graft comprising cancer cells into the tissue explant, therein making a tissue composition
(iii) contacting the tissue composition with the candidate drug;
(iv) measuring cancer cell death within the tissue graft after treatment with the candidate drug for a period of time, thereby determining the cytotoxic effect of the candidate drug on cancer cells.

In some embodiments, the disclosure provides a system for use in a high throughput screening assay. In some embodiments, the system for a high throughput screening assay comprises:
(i) a substrate comprising a plurality of microwells,
(ii) a tissue explant described herein, and
(iii) a tissue graft described herein placed within the tissue explant;
thereby allowing measurement of an effect on the tissue graft through the tissue explant.

In some embodiments, the disclosure provides a system for use in a high throughput cytotoxicity screening assay. In some embodiments, the system for the high throughput cytotoxicity screening assay comprises
(i) a substrate comprising a plurality of microwells,
(ii) a tissue explant described herein, and
(iii) a tissue graft described herein placed within the tissue explant;
thereby allowing measurement of cytotoxicity toward the tissue graft through the tissue explant.

In some embodiments, the disclosure provides a system for use in a high throughput colorectal cancer cytotoxicity screening assay. In some embodiments, the system for the high throughput colorectal cancer cytotoxicity screening assay comprises
(i) a substrate comprising a plurality of microwells,
(ii) a tissue explant comprising epithelium from a large mammalian gastrointestinal tract, wherein the gastrointestinal tract epithelium comprises epithelial cells having a polarity in the tissue explant, and
(iii) a tumorigenic tissue graft placed within the tissue explant;
thereby allowing measurement of cytotoxicity toward the tumorigenic tissue graft through the tissue explant.

In some embodiments, a compound of interest is added to the tissue composition followed by detection of the compound at both the basolateral and luminal surfaces of the tissue explant as well as the tissue graft. Presence of the compound within the graft demonstrates penetration of the compound into tumor tissue. A person of ordinary skill in the art can readily determine the concentration of a compound using a variety of methods, for example, spectrophotometric analysis, high performance liquid chromatography with spectrophotometric detection or liquid chromatography-mass spectrometry. In some embodiments, the candidate agent is radiolabeled, allowing for detection in the receiver chamber and within the tissue.

In some embodiments, the tissue composition is treated with a compound of interest for at least 1 hour, at least 5 hours, at least 10 hours, at least 15 hours, at least 20 hours, at least 24 hours, at least 36 hours, or at least 48 hours. In some embodiments, the tissue composition is treated with a compound for 24 hours.

In some embodiments, the tissue graft expresses GFP. In some embodiments, cytotoxicity is measured by quantifying GFP positive cells in the graft. GFP positive cells are quantified using methods known in the art. Examples include but are not limited to flow cytometry analysis and immunohistochemistry.

In some embodiments, the IC₅₀ value of a drug is determined using the methods described herein.

### Predicting or Determining Drug Transport Mechanisms

In some aspects of the disclosure, the tissue composition described herein is useful for capturing the effect of drug transport on the efficacy of cancer-therapeutics. In further aspects of the disclosure, the tissue composition described herein is useful for determining drug transport mechanisms.

Overexpression of drug transporters in tumors contributes to drug resistance in cancer (Dalton, W. S. The tumor microenvironment: focus on myeloma. Cancer Treat Rev 29 Suppl 1, 11-19 (2003); Jean, C., Gravelle, P., Fournie, J. J. & Laurent, G. Influence of stress on extracellular matrix and integrin biology. Oncogene 30, 2697-2706 (2011)). P-gp inhibitors (e.g. drug transport inhibitors) are known for use in the clinic to improve tumor penetration. In some embodiments, the disclosure provides methods of screening cytotoxic drugs in combination with drug transport inhibitors to identify suitable combinations for therapy.

In some embodiments, the gastrointestinal toxicity of a compound or composition of interest is determined by: contacting the tissue composition described herein with the compound or composition; waiting a sufficient period of time; and conducting a toxicity assay. Methods for analyzing the toxicity of a compound or composition are known to those of skill in the art and further described herein. For example, in some embodiments, the toxicity assay is a resazurin-based viability assay. Resazurin is an oxidation-reduction indicator, wherein it is irreversibly reduced to the pink colored and highly red fluorescent resorufin in metabolizing cells. In some embodiments, the toxicity assay is a Live/Dead assay. In some embodiments, the toxicity assay is an alamarBlue® assay. In some embodiments, toxicity is determined by measuring the protein expression of apoptosis markers, such as cleaved caspase 3 and cleaved lamin A/C. In some embodiments, toxicity is determined by measuring the protein expression of DNA damage markers, such has histone H2A phosphorylation. Methods for analyzing protein expression are known to those of skilled in the art and described herein.

In some embodiments, the disclosure provides methods for determining the effect of a drug transporter on efficacy of a test cytotoxicity compound. In some embodiments, effect of a drug transporter is determined by modifying expression of a drug transporter in a tissue composition described herein, contacting the tissue composition with a cytotoxic compound of interest, determining absorption of the compound, and comparing absorption of the compound in a tissue composition with and without a modified drug transporter. Methods for modifying expression of the drug transporter are described infra.

In some embodiments, the disclosure provides methods for identifying adjuvants to improve drug penetration into the tumor microenvironment. In some embodiments, methods for identifying an adjuvant to improve drug penetration into the tumor microenvironment comprises:
(i) providing a tissue explant;
(ii) placing a tumorigenic tissue graft comprising cancer cells into the tissue explant, therein making a tissue composition;
(iii) contacting the tissue composition with a compound of interest formulated with or without an adjuvant of interest;
(iv) measuring cancer cell death within the tissue graft after treatment with compound of interest formulated with or without the adjuvant of interest for a period of time; and
(v) comparing the amount of cancer cell death between tissue compositions treated with the compound of interest formulated with or without the adjuvant
thereby determining whether the adjuvant improved drug penetration.

In some embodiments, the method for identifying an adjuvant to improve drug penetration comprises using a machine learning algorithm as described herein.

In some embodiments, methods for identifying an adjuvant to improve drug penetration into the tumor microenvironment comprises:
(i) providing a gastrointestinal tissue explant;
(ii) placing a tumorigenic tissue graft comprising cancer cells into the tissue explant, therein making a tissue composition;
(iii) contacting the tissue composition with a compound of interest formulated with or without an adjuvant of interest;
(iv) measuring cancer cell death within the tissue graft after treatment with compound of interest formulated with or without the adjuvant of interest for a period of time; and
(v) comparing the amount of cancer cell death between tissue compositions treated with the compound of interest formulated with or without the adjuvant thereby determining whether the adjuvant improved drug penetration.

In some embodiments, methods for identifying an adjuvant to improve drug penetration into the tumor microenvironment comprises:
(i) providing a gastrointestinal tissue explant;
(ii) placing a tumorigenic colorectal cancer organoid comprising cancer cells into the tissue explant, therein making a tissue composition;
(iii) contacting the tissue composition with a compound of interest formulated with or without an adjuvant of interest;
(iv) measuring cancer cell death within the organoid after treatment with compound of interest formulated with or without the adjuvant of interest for a period of time; and
(v) comparing the amount of cancer cell death between tissue compositions treated with the compound of interest formulated with or without the adjuvant
thereby determining whether the adjuvant improved drug penetration.

In some embodiments, the disclosure provides methods for high-throughput screening for analyzing drug-transport mechanisms. In some embodiments, the tissue composition is contacted with a substrate, wherein the substrate comprises a plurality of microwells, wherein the tissue composition is contacted with a formulation library comprising a compound of interest and an excipient, wherein drug-transport mechanisms are elucidated, and wherein drug-transport mechanisms are used to identify a formulation for modifying drug-transport therapies.

In some embodiments, the tissue composition comprises a gastrointestinal tissue explant, wherein the tissue composition is contacted with a substrate, wherein the substrate comprises a plurality of microwells, wherein the tissue composition is contacted with a formulation library comprising a compound of interest and an excipient, wherein drug-transport mechanisms are elucidated, and wherein drug-transport mechanisms are used to identify a formulation for modifying drug-transport therapies.

In some embodiments, the tissue composition comprises a gastrointestinal tissue explant and a tumorigenic graft placed within the explant, wherein the tissue composition is contacted with a substrate, wherein the substrate comprises a plurality of microwells, wherein the tissue composition is contacted with a formulation library comprising a compound of interest and an excipient, wherein drug-transport mechanisms are elucidated, and wherein drug-transport mechanisms are used to identify a formulation for modifying drug-transport therapies.

In some embodiments, the tissue composition comprises a gastrointestinal tissue explant and a tumorigenic colorectal cancer organoid placed within the explant, wherein the tissue composition is contacted with a substrate, wherein the substrate comprises a plurality of microwells, wherein the tissue composition is contacted with a formulation library comprising a compound of interest and an excipient, wherein drug-transport mechanisms are elucidated, and wherein drug-transport mechanisms are used to identify a formulation for modifying drug-transport therapies.

In some embodiments, the library comprises approved and/or experimental drugs. In some embodiments, the library comprises approved and/or experimental drugs conjugated to biologically active or inactive molecules. In some embodiments, a drug library is commercially available.

### Machine Learning Algorithm to Identify Drug-Transporter Interactions

In some embodiments, the methods and compositions provided herein comprise a machine learning algorithm to identify drug-transporter interactions. In some embodiments, the identification of drug-transporter interactions allows for identification of adjuvants to increase absorption of a drug. In some embodiments, the adjuvant improves penetration of a cytotoxic drug into a tumor microenvironment.

In some embodiments, a machine learning algorithm is generated by curating a training dataset mined from known databases. In some embodiments, the databases are DrugBank 5.0, Metrabase, and NIH screen NCI-60. In some embodiments, a random forest machine learning model is used to predict the substrate relationships within a dataset based on chemical and physiochemical features of the substrates and non-substrates.

In some embodiments, a machine learning algorithm is used to determine an interaction between a candidate drug and drug transporter. In some embodiments, the machine learning algorithm identifies whether a candidate drug is a substrate of a drug transporter. In some embodiments, a machine learning algorithm determines whether a candidate drug interacts with a group of drug transporters.

In some embodiments, the machine learning algorithm provides information on likely transporter-substrate relationships. In some embodiments, the machine learning algorithm predicts transporter-substrate relationships. In some embodiments, a machine learning algorithm identifies adjuvants for decreasing drug transporter activity through direct inhibition or substrate competition.

In some embodiments, the machine learning algorithm classifies investigational drugs into categories of substrates for individual drug transporters or combinations of drug transporters.

In some embodiments, a machine learning algorithm identifies drug-drug transporter interaction(s) which can then be validated using any of the systems, methods and compositions described herein.

In some embodiments, candidate drugs identified by machine learning are prioritized by commercial availability for validation using any of the systems, methods and compositions described herein. In some embodiments, candidate drugs identified by machine learning are prioritized by translational applicability for validation using any of the systems, methods and compositions described herein. In some embodiments, candidate drugs identified by machine learning are prioritized by commercial availability and translational applicability for validation using any of the systems, methods and compositions described herein.

In some embodiments, a machine learning algorithm identifies drug-transport inhibitors as effective adjuvants to improve cancer targeting. In some embodiments, a machine learning algorithm identifies drug-transport inhibitors as effective adjuvants to improve 5-fluorouracil (5-FU), irinotecan, oxaliplatin, regorafenib, or capecitabine cancer targeting. In some embodiments, a machine learning algorithm identifies drug-transport inhibitors as effective adjuvants to improve irinotecan cancer targeting.

### High-Throughput Screening

In some aspects of the disclosure, methods and culture systems are provided for screening candidate agents (e.g., cytotoxic drugs and/or adjuvants) in a high-throughput format. By “high-throughput” or “HT”, it is meant the screening of large numbers of candidate agents or candidate cells simultaneously for an activity of interest. By large numbers, it is meant screening 20 or more candidates at a time, e.g. 40 or more candidates, e.g. 100 or more candidates, 200 or more candidates, 500 or more candidates, or 1000 candidates or more.

In some embodiments, the high throughput screen is formatted based upon the numbers of wells of the tissue culture plates used, e.g. a 24-well format, in which 24 candidate agents (or less, plus controls) are assayed; a 48-well format, in which 48 candidate agents (or less, plus controls) are assayed; a 96-well format, in which 96 candidate agents (or less, plus controls) are assayed; a 384-well format, in which 384 candidate agents (or less, plus controls) are assayed; a 1536-well format, in which 1536 candidate agents (or less, plus controls) are assayed; or a 3456-well format, in which 3456 candidate agents (or less, plus controls) are assayed.

In some embodiments, the disclosure provides methods for high-throughput screening for analyzing drug transport mechanisms. In some embodiments, the disclosure provides methods for high-throughput screening for analyzing cytotoxicity of a candidate drug or drugs toward a tissue graft (e.g., tumor tissue). In some embodiments, the tissue composition (i.e. the tissue explant and graft) is contacted with a substrate, wherein the substrate comprises a plurality of microwells, wherein the tissue composition is contacted with a formulation library comprising a compound of interest and an excipient, wherein absorption of the compound of interest is determined, and wherein results of absorption are compared to identify a formulation for drug absorption. In some embodiments, the tissue composition is contacted with a substrate, wherein the substrate comprises a plurality of microwells, wherein the tissue composition is contacted with a formulation library comprising a compound of interest and an excipient, wherein cytotoxicity of the compound of interest toward the tumor tissue is determined, and wherein results of cytotoxicity are compared to identify a formulation for drug therapy.

In some embodiments, the formulation library is a library of GRAS-based excipients that are either known absorption enhancers or have an unknown effect on intestinal absorption.

### Compounds of Interest

Compounds of interest are biologically active agents that encompass numerous chemical classes, organic molecules, which may include organometallic molecules, inorganic molecules, genetic sequences, etc. One aspect of the disclosure is to evaluate the absorption of candidate drugs and identify optimum formulations for absorption. Another aspect of the disclosure is to analyze the local effect of an active pharmaceutical ingredient (A-PI) on the tissue. For example, the effect can include, but is not limited to, local tissue toxicity, genetic modification of tissue, temporary change of tissue permeability, drug transporter/metabolizing enzyme inhibition, modulation of mucus or microbiome, and modulation of hormone production and/or secretion. Another aspect of the disclosure is to evaluate the effect of combinations of APIs.

Compounds of interest comprise functional groups necessary for structural interaction with proteins, particularly hydrogen bonding, and typically include at least an amine, carbonyl, hydroxyl or carboxyl group, frequently at least two of the functional chemical groups. The compounds often comprise cyclical carbon or heterocyclic structures and/or aromatic or polyaromatic structures substituted with one or more of the above functional groups. Compounds of interest are also found among biomolecules, including peptides, polynucleotides, saccharides, fatty acids, steroids, purines, pyrimidines, derivatives, structural analogs or combinations thereof. Included are pharmacologically active drugs, genetically active molecules, etc. In some embodiments, compounds of interest include chemotherapeutic agents, anti-inflammatory agents, hormones or hormone antagonists, ion channel modifiers, and neuroactive agents.

Compounds, including candidate agents, are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. In some embodiments, the library comprises approved and/or experimental drugs. In some embodiments, the library comprises approved and/or experimental drugs conjugated to biologically active or inactive molecules. In some embodiments, a drug library is commercially available.

In some embodiments, candidate agents can also be genetic agents, such as polynucleotides and analogs thereof, which are tested in the screening assays described herein by addition of the genetic agent to the tissue composition. The introduction of the genetic agent can result in an alteration of the total genetic composition of the cells within the tissue composition. Genetic agents such as DNA can result in an experimentally introduced change in the genome of a cell, generally through the integration of the sequence into a chromosome. Genetic changes can also be transient, where the exogenous sequence is not integrated but is maintained as episomal agents. Genetic agents, such as antisense oligonucleotides, can also affect the expression of proteins without changing the cell's genotype, by interfering with the transcription or translation of mRNA. Genetic agents, such as short interfering RNA (siRNA) or short hairpin (shRNA), can effect expression of proteins without changing the cell's genotype by mediated the degradation of the mRNA it binds to. The effect of a genetic agent is to increase or decrease expression of one or more gene products in the cell.

The tissue composition described herein is useful for predicting the absorption, toxicity and/or endocrine stimulation of a variety of agent types. In some embodiments, candidate agents are small molecules (e.g., doxycycline). In some embodiments, candidate agents are small molecule drugs. In some embodiments, candidate agents are biologics, including peptide drugs (e.g., oxytocin) and protein drugs (e.g., insulin). In some embodiments, candidate agents are antisense oligonucleotides.

In some embodiments, candidate agents are known drugs classified by the FDA's Biopharmaceutics Classification System (BCS), which takes into account three major factors that govern the rate and extent of drug absorption from immediate release (IIR) solid oral dosage forms: dissolution, solubility and intestinal permeability. BCS Class I refers to high solubility and high permeability. BCS Class II refers to low solubility and high permeability. BCS Class II refers to high solubility and low permeability. BCS Class IV refers to low solubility and low permeability.

### Inhibitor of P-gp for Improving Drug Penetration

In some embodiments, the methods, compositions, and systems described herein identify P-gp inhibitors for improving drug penetration. P-glycoprotein 1(P-gp) is a plasma membrane protein that transports molecules (e.g. drugs) across the cell membrane. P-gp is the most predominant and well-characterized drug transporter known to-date and its overexpression is associated with both de novo and acquired resistance to chemotherapy. Accordingly, in some embodiments, the disclosure provides P-gp inhibitors for improving drug penetration. In some embodiments, the disclosure provides P-gp inhibitors for improving drug penetration to a tumor microenvironment. In some embodiments, the disclosure provides P-gp inhibitors for improving drug penetration of a chemotherapeutic agent to a tumor microenvironment. In some embodiments, a P-gp inhibitor improves drug-penetration. In some embodiments, a chemotherapeutic agent is formulated with a P-gp inhibitor. In some embodiments, the P-gp inhibitor is cinobufagin (CBF). In some embodiments, the P-gp inhibitor is lapatinib. In some embodiments, the P-gp inhibitor reduces the IC₅₀ of a chemotherapeutic during co-treatment.

In some embodiments, a tissue composition described herein is contacted with a P-gp inhibitor and a compound of interest simultaneously or sequentially. In some embodiments, a tissue composition described herein is contacted with a compound of interested formulated with a P-gp inhibitor to improve penetration of the compound of interest.

In some embodiments, methods for identifying P-gp inhibitors as functional adjuvants to improve drug penetration into the tumor microenvironment comprises:
(i) providing a tissue explant;
(ii) placing a tumorigenic graft comprising cancer cells into the tissue explant, therein making a tissue composition;
(iii) contacting the tissue composition with a compound of interest formulated with a known or predicted P-gp inhibitor;
(iv) measuring cancer cell death within the tissue graft after treatment with compound of interest formulated with the known or predicted P-gp inhibitor for a period of time; and
(v) comparing the amount of cancer cell death between tissue compositions treated with the compound of interest formulated with the known or predicted P-gp inhibitor,
thereby determining whether the known or predicted P-gp inhibitor improves drug penetration of the compound of interest.

In some embodiments, methods for identifying P-gp inhibitors as functional adjuvants to improve drug penetration into the tumor microenvironment comprises:
(i) providing a gastrointestinal tissue explant;
(ii) placing a gastrointestinal tumorigenic graft comprising cancer cells into the tissue explant, therein making a tissue composition;
(iii) contacting the tissue composition with a compound of interest formulated with a known or predicted P-gp inhibitor;
(iv) measuring cancer cell death within the tissue graft after treatment with compound of interest formulated with the known or predicted P-gp inhibitor for a period of time; and
(v) comparing the amount of cancer cell death between tissue compositions treated with the compound of interest formulated with the known or predicted P-gp inhibitor,
thereby determining whether the known or predicted P-gp inhibitor improves drug penetration of the compound of interest.

### Kits

In some aspects, the disclosure provides a kit comprising a tissue explant and graft as described herein. In some embodiments, a kit includes a tissue explant described herein, a graft, and optionally a substrate, and instructions for use. The kits may comprise, in a suitable container, a tissue explant described herein, and optionally a substrate, and various buffers, reagents, enzymes and other standard ingredients well known in the art. In some embodiments, the kit comprises a tissue explant and graft described herein, a substrate, and one or more formulations. In some embodiments, the formulation is a GRAS (Generally Recognized as Safe)-based excipient. In some embodiments, the kit comprises a library of formulations. In some embodiments, the substrate comprises plates for interfacing with the tissue explant and graft, and cover films to seal one of the plates.

In some embodiments, the kit comprises a tissue explant and graft described herein and a substrate comprising plates for interfacing with the tissue explant and cover films to seal one of the plates, wherein the substrate is compatible with a robotic arm. Such containers may include injection or blow-molded plastic containers into which the desired components are retained. Containers and/or kits can include labeling with instructions for use and/or warnings.

### Definitions

Terms used in the claims and specification are defined as set forth below unless otherwise specified.

It must be noted that, as used in the specification and the claims, the singular forms “a,” “an,” and “the” include plural referents unless the context clearly dictates otherwise.

As used herein, “about” will be understood by persons of ordinary skill and will vary to some extent depending on the context in which it is used. If there are uses of the term which are not clear to persons of ordinary skill given the context in which it is used, “about” will mean up to plus or minus 10% of the particular value.

As used herein, “architecture” refers to a tissue structure including the specific cell types within the tissue and the extracellular matrix surrounding the cells. In some embodiments, an ex vivo composition of the disclosure comprises a tissue explant comprising epithelium from a mammalian source tissue having an architecture and the tissue explant substantially maintains all or a substantial portion of the architecture (e.g., the in vivo architecture) of the tissue from which it was derived (e.g., small intestine). For example, when the source tissue comprises intestinal epithelium having an architecture comprising epithelial cells having a polarity, the tissue explant comprises the architecture of the source tissue from which it was derived (e.g., small intestine) in the ex vivo composition, and use thereof. In some embodiments, the tissue explant described herein mimics in vivo architecture. In some embodiments, the tissue explant described herein mimics in vivo architecture of a source tissue. In some embodiments, the tissue explant described herein mimics the in vivo architecture of the small intestine. In some embodiments, a tissue explant mimics in vivo architecture wherein it comprises one or more physical structures representative of the in vivo tissue from which it was derived. In some embodiments, tissue architecture is based on cell-cell interactions. In some embodiments, tissue architecture is based on cell-extracellular matrix interactions. For example, wherein the tissue explant is derived from the small intestine, it mimics the in vivo architecture of the small intestine by comprising at least one structure of the small intestine from the tissue from which it was derived, for example, by comprising intact crypts, intestine epithelium, circular muscular layer and/or villi, or any combination of the foregoing. In some embodiments, a tissue explant mimics in vivo architecture by comprising one or more or a majority of the structures of the tissue from which it was derived, for example by comprising intact crypts, intestine epithelium, circular muscular layer and/or villi, or any combination of the foregoing. In some embodiments the tissue explant comprises intact crypts, intestine epithelium, circular muscular layer, and villi from the tissue from which it was derived (e.g., a large, non-human, mammalian gastrointestinal tract or a human gastrointestinal tract). In some embodiments, determination of the architecture of the tissue explant and whether it mimics the in vivo architecture of the tissue from which it is derived can be determined by standard techniques known in the art, for example, by comparing the structure of the tissue explant in the ex vivo composition of the disclosure by methods described herein (e.g., histological staining) with images or information available to those of skill in the art (e.g., previously obtained images of the tissue from which the explant is derived). In some embodiments, comparisons are made between tissue explants cultured ex vivo and tissue explants freshly excised.

As used herein, “a basolateral surface” refers to the orientation of the tissue explant when contacted with a substrate, such that the tissue explant comprises apical/luminal-basolateral polarity. In some embodiments, the basolateral surface is opposite of the apical surface, i.e., the luminal surface.

As used herein, “contacting” refers to either placing a substrate on a tissue explant described herein (or causing a tissue explant to come in contact with a substrate), or placing a compound of interest on an ex vivo composition described herein (or causing a compound of interest to come in contact with an ex vivo composition).

As used herein, “detecting”, “detect” and “detection” refer to the identification and/or quantification of a compound of interest (e.g., drug, agent, etc.) in a sample. In some embodiments, detecting comprises determining the absence or presence of a compound of interest in a sample. In some embodiments, detecting comprises quantifying a compound of interest in a sample. In some embodiments, detecting comprises identifying and/or quantifying a compound of interest in a sample at different time points. In some embodiments, detecting comprises identifying and/or quantifying a compound of interest in a first sample and in a second sample.

As used herein, “drug absorption” or “drug perfusion” refers to the movement of drug into the bloodstream and through tissues following administration, as well as movement of drug through the tissue explant following contact of drug with the tissue explant. Drug absorption or perfusion is determined by the drug's physicochemical properties, formulation, and route of administration.

As used herein, “drug dissolution” refers to the rate a dosage form (e.g., tablet) of a drug dissolves in the fluids of the gastrointestinal tract prior to absorption into the systemic circulation.

As used herein, “drug transporter” refers to proteins that move drugs across the cell membrane. In general, drug transporters are divided into two major superfamilies: ATP-binding cassette (ABC) family and solute carrier (SLC) family. The ABC transporters are primary active transporters that utilize the energy from ATP hydrolysis to transport substrates (e.g., drugs) across the membrane. SLC transporters can either be facilitative transporters, which transport their substrates down the gradient across the membrane, or secondary active transporters, which transport their substrates against the gradient across the membrane by coupling a downhill transport of another substrate.

As used herein, “exogenous” refers to molecules or compositions originating or produced from outside an organism, tissue or cell.

As used herein, the “extracellular matrix” refers to a complex non-cellular three-dimensional macromolecular network composed of collagens, proteoglycans/glycosaminoglycans, elastin, fibronectin, laminins, and several other glycoproteins. These molecules are secreted locally by cells and remain closely associated with them to provide structural, adhesive and biochemical signaling support.

As used herein, “ex vivo” refers to a condition that takes place outside an organism. In some embodiments, ex vivo refers to experimentation or measurements done in or on a tissue from an organism in an external environment.

As used herein, “gastrointestinal tract” refers to the complete system of organs and regions that are involved with ingestion, digestion, and excretion of food and liquids. This system generally consists of, but is not limited to, the mouth, esophagus, stomach and or rumen, intestines (small and large), cecum (plural ceca), fermentation sacs, and the anus.

As used herein, the term “graft” refers to a population of cells or tissue derived from any in vivo or in vitro source. For example, in some embodiments, the graft is derived from tissue from a subject and comprises a population of cells from said tissue or a piece of said tissue. In some embodiments, the graft is derived from an immortalized cell line. In some embodiments, the graft comprises a population of cells (e.g., immortalized cells or cells derived from a subject) cultured on a biocompatible scaffold to provide a three-dimensional structure.

As used herein, “high-throughput” refers to the parallelization of experiments. Specifically, several experiments can be run simultaneously as opposed to single experiments carried out one after another. In some embodiments, high-throughput experiments are carried out using automated techniques.

As used herein, “intestinal cells” refers to cells that make up the mammalian intestinal epithelium. The mammalian intestinal epithelium of the gastrointestinal tract has a well-defined organizational structure. The epithelium can be divided into two regions, a functional region that houses differentiated cells (villi) and a proliferative region (crypts of Lieberkuhn) that represents the epithelium stem cell niche. Multipotent epithelium stem cells reside in the crypts and give rise to four principal epithelial lineages: absorptive enterocytes, mucin secreting goblet cells, peptide hormone secreting enteroendocrine cells, and Paneth cells.

As used herein, “intestine” refers to the mammalian small intestine and mammalian large intestine.

As used herein, “intestinal stem cells,” used interchangeably with “epithelial stem cells” refers to stem cells that have the potential to proliferate and differentiate into intestinal epithelial cells. Multipotent epithelial stem cells give rise to various epithelial lineages, and may give rise to all intestinal epithelial lineages, which include: absorptive enterocytes, mucin secreting goblet cells, peptide hormone secreting enteroendocrine cells, and Paneth cells.

As used herein, “in vitro” refers to processes performed or taking place outside of a living organism. In some embodiments, the processes are performed or take place in a culture dish.

As used herein, “in vivo” refers to processes that occur in a living organism.

As used herein, “lamina propria” refers to a thin layer of loose connective tissue, or dense irregular connective tissue, which lies beneath the epithelium and together with the epithelium constitutes the mucosa.

As used herein, “lamina muscularis,” “lamina muscularis mucosae” and “muscularis mucosae” refer to a thin layer of muscle of the gastrointestinal tract located outside the lamina propria and separating it from the submucosa.

As used herein, “large mammal” refers to a species in which normal mature adults of either sex may attain a body mass of at least one kilogram. In some embodiments, a large mammal is an ungulate (i.e., hoofed mammals such as pigs, cows, goats, sheep, horses, donkeys, deer, antelopes and the like). In some embodiments, a large mammal is livestock (i.e., mammals raised for agricultural purposes such as pigs, cows, goats, sheep, horses, rabbits, and the link, and/or as beasts of burden such as donkeys, horses, elephants, camels, llamas, and the like). In some embodiments, a large mammal is a human.

As used herein, “luminal surface” refers to the orientation of the tissue explant when contacted with a substrate, such that the tissue explant comprises apical/luminal-basolateral polarity. In some embodiments, the luminal surface is opposite of the basolateral surface.

As used herein, “maintained in culture” refers to the continued application of conditions that are required for the growth or survival of a specific cell type in an artificial environment. In some embodiments the artificial environment includes substrate or medium that supplies the essential nutrients (e.g., amino acids, carbohydrates, vitamins, minerals), growth factors, hormones, gases (e.g., O2, CO2), and physicochemical environment (e.g., pH, osmotic pressure, temperature). In some embodiments, the tissue explant described herein is maintained in culture for up to 1 week. In some embodiments, the tissue explant described herein is maintained in culture for up to 2 weeks. In some embodiments, the tissue explant described herein is maintained in culture for up to 3, 4, 5, 6, 7, 8, 9, 10, 11, 12, 13, 14, 15, 16 or 17 weeks. In some embodiments, the tissue explant described herein is maintained in culture for up to 18 weeks.

As used herein, “drug metabolizing enzyme”, “DME” and “metabolizing enzyme” refer to polypeptides responsible for metabolizing a vast array of xenobiotic chemicals, including drugs, carcinogens, pesticides, pollutants and food toxicants, as well as endogenous compounds, such as steroids, prostaglandins and bile acids. Metabolic biotransformation of chemicals by DMEs form more hydrophilic, polar entities, which enhance their elimination from the body and lead to compounds that are generally pharmacologically inactive and relatively nontoxic. In some embodiments, metabolic biotransformation can lead to the formation of metabolites with pharmacological activity. Xenobiotics are metabolized by four different reactions: oxidation, reduction, hydrolysis and conjugation. Oxidation, reduction and hydrolysis are referred to as Phase I reactions, and conjugation is referred to as a Phase II reaction. Oxidative Phase I DMEs include cytochrome P450s (CYPs or P450s), Flavin-containing monooxygenases (FMOs), monoamine oxidase (MAOs), and xanthine oxidase/aldehyde oxidase (XO/AO). Conjugative Phase II DMEs include uridine 5′-diphospho (UDP)-glucuronosyltransferases (UGTs), sulfotransferases (SULTs), glutathione S-transferases (GSTs), N-acetyltransferase (NATs), and methyl (N-methyl-, thiomethyl-, and thiopurinemethyl-) taransferases. Of the DMEs involves in the metabolism of drugs, the dominant players are P450 enzymes, followed by UGTs and esterases. Accordingly, in some embodiments, the tissue explant described herein comprises Phase I and Phase II metabolizing enzymes. In some embodiments, the tissue explant described herein comprises a cytochrome P450 enzyme and a UGT enzyme.

As used herein, “modulation of gene expression” refers to changes in the induction or repression of a gene. Mechanisms that are involved with the gene regulation include structural and chemical changes to the genetic material, binding of proteins to specific DNA elements to regulate transcription, and/or mechanisms that modulate translation of mRNA. In some embodiments, gene expression of the tissue explant described herein is modulated. In some embodiments, gene expression of at least one drug transporter present in the tissue explant described herein is modulated. In some embodiments, gene expression of at least one metabolizing enzyme present in the tissue explant described herein is modulated.

As used herein, “mucus” refers to a viscid secretion that is usually rich in mucins and is produced by mucous membranes which it moistens and protects. In some embodiments, the tissue explant described herein produces mucus.

As used herein, “muscularis externa” refers to the circular muscle layer and the longitudinal muscle layer, which separate the submucosa from the subserous layer. In some embodiments, the tissue explant described herein comprises an intact muscularis externa. In some embodiments, the tissue explant described herein comprises only the circular muscle layer.

The term “organoid” refers to a miniaturized version of an organ that is produced in vitro in three dimensions. An organoid shows microanatomy and cellular function resembling that of native tissues in vivo. Typically, an organoid comprises multiple organ-specific cell types, wherein said cell types are spatially organized in a defined manner, in the case of neural organoids typically in layers. Generally, said defined spatial organization of multiple cell types is a result of self-organization occurring during the formation of the organoid. Organoids comprise distinct cell types that interact spatially and/or functionally with each other, preferably in a self-organized matrix. The term “self-organized matrix” refers to the spatial arrangement of cells with different cellular function and identity such that they resemble in part or entirely the cellular arrangement found in native tissues in vivo. The term “organoid” includes colonoids and enteroids. Enteroids are three-dimensional culture constructs propagated from stem cells from intestinal crypts isolated from surgical specimens, biopsies, autopsy, or a combination thereof.

As used herein, “oral bioavailability” refers to the degree to which a drug or other substance becomes available to a target tissue after oral administration. Bioavailability is related to the physiochemical properties of a drug or other substance, e.g., dissolution, membrane transport, chemical stability, etc., as well as the interactions with the host, e.g., metabolic fate, distribution and clearance. In some embodiments, the tissue explant described herein predicts the oral bioavailability of a drug or other substance of interest.

As used herein, “Pearson product-moment correlation coefficient” or “Pearson correlation coefficient” refers to a measurement of the strength of a linear association between two variables and is denoted by “r”.

As used herein, “planar contact” refers to the placement of the tissue explant on a substrate, such that the tissue explant interacts with a two-dimensional surface of the substrate. Planar contact can be determined by methods known to those of skill in the art. For example, a method for analyzing planar contact comprises (i) contacting a tissue explant with a solution comprising a marker (e.g., dye) to stain the tissue and (ii) detecting the stain on the surface of the tissue by photographic inspection, spectrophotometrically or by laser scanner. The tissue explant is considered to be in planar contact with the substrate if there is no significant difference in variability of the marker within the area contacted with the substrate compared to an equivalent area of non-mounted tissue completely immersed in the solution comprising the marker. In another example, planar contact is determined by (i) coating the substrate with a marker that forms a uniform layer on the surface of the substrate; (ii) contacting the substrate with the tissue explant; and (iii) analyzing the resulting stain on the tissue explant once it is separated from the substrate by visual inspection. The tissue explant is considered to be in planar contact with the substrate if the tissue shows a regular pattern of markings across the entire tissue that correlate with the pattern of the substrate.

As used herein “polarity” refers to the organization of the cell membrane with associated proteins, along with the arrangement of the cytoskeleton and organelles within the cytoplasm. For example, epithelial cells are organized along a cellular axis that extends from the apical side facing an external lumen to the basal side facing either the extracellular matrix or adjacent cells. In addition to the apical-basal axis of polarity, epithelial cells are often oriented within the plane of the tissue along a proximal-distal axis, referred to as “tissue polarity” or “planar polarity. In some embodiments, the apical-basal axis of polarity of epithelial cells is maintained in the tissue explant following removal from the source tissue. In some embodiments, the apical-basal axis of polarity of epithelial cells is maintained in the tissue explant following contact with the substrate. In some embodiments, the apical-basal axis of polarity of epithelial cells is maintained in the ex vivo composition following use in the methods as described herein. In some embodiments, the proximal-distal axis of polarity is maintained in the tissue explant following removal from the source tissue. In some embodiments, the proximal-distal axis of polarity of epithelial cells is maintained in the tissue explant following contact with the substrate. In some embodiments, the proximal-distal axis of polarity of epithelial cells is maintained in the ex vivo composition following use in the methods as described herein. In some embodiments, both the apical-basal axis and proximal-distal axis of polarity are maintained in the tissue explant following contact with the substrate. In some embodiments, the apical-basal axis and the proximal-distal axis of polarity of epithelial cells is maintained in the tissue explant following contact with the substrate. In some embodiments, the apical-basal axis and proximal-distal axis of polarity of epithelial cells is maintained in the ex vivo composition following use in the methods as described herein. Methods of determining polarity are known to those of skill in the art. A review of such methods can be found in Chapter 7 of Cell Polarity and Morphogenesis (Academic Press, 2017, herein incorporated by reference in its entirety). In some embodiments, polarity of the tissue explant described herein is analyzed by visual (e.g., microscopic) inspection. For example, in some embodiments, the tissue explant described herein comprises two or more genetically distinct cell populations and polarity can be determined by expression of a labeled protein in only a subset of cells and subsequently visualized by microscopic techniques. In some embodiments, immunohistochemistry and live images of fluorescent reports are used to visualize proteins in their tissue context and evaluate their distribution. In some embodiments, cell polarization is quantified by analyzing protein localization in fluorescent images and calculating the ratio of fluorescence intensity between regions where the protein is present and regions where it is weakly localized or absent. The fluorescence ratio provides a quantitative measure of asymmetric protein distribution. See Marcinkevicius, E., et al. J. Biol. 2009, Vol. 8(12): 103, herein incorporated by reference in its entirety. In some embodiments, the fluorescence ratio is normalized by choosing appropriate analysis settings and incorporating internal controls, as described by Shimoni, R., et al. PLos ONE 2014, Vol. 9(6): e99885, herein incorporated by reference in its entirety.

As used herein, “reusable” refers to the ability of a tissue explant to be subjected to more than one experiment in succession.

As used herein, “responsive” refers to a reaction elicited by a stimulus. In some embodiments, the tissue explants described herein are responsive to a stimulus. In some embodiments, the tissue explant described herein is responsive to glucose. In some embodiments, increased GLP-1 activity (e.g., increased concentration of active GLP-1 7-36) indicates the tissue explant is responsive to glucose. In some embodiments, when the apical side of the tissue explant is contacted with glucose, GLP-1 activity is increased. In some embodiments, modulation of gut hormones and/or tissue behavior indicates the tissue explant is responsive to glucose. Methods for measuring gut hormones and tissue behavior are described herein.

As used herein, “substrate” refers to a surface or layer that underlies something, for example, a cell, cell culture, cell culture material, etc., or on which processes occur. In some embodiments, a substrate is a surface or material on which an organism lives, grows, and/or optionally obtains nourishment. The term “substrate” also refers to a surface or layer, e.g., a base surface or layer, on which another material is deposited. Exemplary substrates include, but are not limited to, glass, silicon, polymeric material, plastic (e.g., tissue culture plastic), etc. Substrates can be slides, chips, wells and the like.

As used herein, “tissue explant” refers to an isolated piece or pieces of tissue. In some embodiments, the tissue explant is isolated from the gastrointestinal tract.

### EXAMPLES

### Materials and Methods

Mice and Swine small intestine tissue harvest: Swine and B6(Cg)-Rag2^{tm1.1Cgn}/J mice (The Jackson Laboratory) were housed in the animal facility at the Koch Institute for Integrative Cancer Research at MIT. All animal studies described in this study were approved by the MIT Institutional Animal Care and Use Committee.

Intestinal organoid isolation and culture: All animal experiments were approved by and performed in accordance with the MIT Committee on Animal Care. Swine were seated with an intramuscular injection of Telazol (5 mg/kg), xylazine (2 mg/kg), and atropine (0.05 mg/kg) via a face mask. After swine were euthanized, 10 cm of the proximal small intestine was removed freshly from the pig and extensively and carefully washed with cold PBS to remove all the residuals. The tissue was stored in cold PBS to prevent degradation until usage. With the washed tissue, scissors were used to open the intestine longitudinally and cut into 1-2 cm small pieces. The tissue was emerged with 50 mL ice-cold PBS with 5 mM EDTA and shaken gently (100 rpm) at 4° C. for an hour. The PBS+EDTA was replaced with 30 mL fresh cold PBS and shaken vigorously for 2-3 minutes to release crypts. Supernatant was filtered with 100 uM cell strainer followed with centrifugation at 300 rpm 4° C. for 5 minutes. The organoid pellet was resuspended in 25% complete culture medium and 75% Matrigel (Corning). 25 uL organoid mixture was plated as a bubble to a 24-well plate. After polymerization, the Matrigel dome was covered with 500 uL complete culture medium, and incubated at 37° C. with 5% CO₂. The organoid was passaged with 1:4 ratio every 7 days with mechanical disruption with PBS.

Organoid Culture and nutrition selection: The complete culture medium (50% L-WRN) was prepared as described previously. Briefly, 50% L-WRN medium was enriched by mixing the supernatant of L-WRN cell (ATCC) medium with Advanced Dulbecco's Modified Eagle medium/F12 in 1:1 ratio. 10 uM Y27632 (Sigma) and 3 uM SB202190 (Sigma), 40 ng/mL EGF (PeproTech), and 100 ug/mL primocin (InvivoGene) were added to 50% L-WRN to obtain the complete culture medium. The basal medium was prepared with the following ingredients: Advanced Dulbecco's Modified Eagle medium/F12, 100 ug/mL penicillin/streptomycin, 10 mM HEPES, 2 mM GlutaMAX, 1xB27, 10 nM gastrin I, 500 nM A83-01, 10 uM Y27632 (Sigma) and 3 uM SB202190 (Sigma), and 1 mM N-acetylcysteine. For APC selection medium, the medium was prepared by adding 50 ng/mL mouse recombinant EGF, 100 ng/mL mouse recombinant noggin into basal medium; P53 selection medium was prepared with 25 uM nutlin-3 in complete culture medium; and for SMAD4 selection medium, the basal medium was supplied with 10 ng/mL human recombinant TGF-β, 100 ng/mL human recombinant BMP4, 100 ng/mL human recombinant Wnt-3A, and 10 uM human recombinant R-spondin-1.

Organoid virus transfection and plasmid transformation: Organoid in every well of 48-well plate was dissociated with physical force in pre-chilled PBS followed with Trypsin digestion at 32° C. for 2 minutes. After neutralization and centrifugation, a single cell was suspended in 450 μL of complete culture medium and plated in a well of 48-well plate. Meanwhile, a solution of 1.5 μg of plasmid was mixed with 4.5 μL of Lipofectamine 2000 or 10 L of lentivirus partical (107 pfu) with 0.5 μL of polybrebe stock solution (10 mg/mL) in Opti-MEM, mixed at room temperature and added into the single cell solution. The plate was centrifuged at 600 g, 32° C. for one hour and immediately put into incubation at a 37° C. CO₂ incubator for 4 hours. After this, the cell was collected by centrifuge and plated on a 48-well plate with matrigel. Organoid was allowed to grow in complete medium for 7 days and selected by nutrition (plasmid transduction) or 2 μg/mL of puromycin (lentivirus transformation) for 7 days.

CRISPR genome engineering: Gene-specific sgRNA oligos were designed from CRISPRko platform of the Broad Institute (https://portals.broadinstitute.org/gpp/public/analysis-tools/sgrna-design) and cloned into the PX458 expression vector (Addgene; SEQ ID NOs: 1-24). The editing efficacy was screened through a T7-endonuclease assay reported previously. Briefly, vector containing the sgRNA was transfected into PK15 cell line with lipofectamine 2000. 48 hours after transfection, the genome DNA was extracted with ZYMO Genomic DNA Extraction Kit. Target region was amplified with PCR and treated with T7 endonuclease followed with TBE electrophoresis analysis. For one well of 48-well organoid, a single cell suspension was obtained by Trypsin-L express digestion at 32° C. for 2 minutes. The reaction was quenched by adding complete culture medium. After aspirate supernatant, the cell pelleted was resuspended in 450 uL complete culture medium and a mixture of 1.5 ug plasmid with 4 uL of lipofectamine in 100 uL Opti-MEM or 100 uL Lenti-eGFP particle (10⁶ PFU/mL) in DMEM (30% FBS). The plate was centrifuged at 32° C. 600 rpm for an hour, then transferred into a 37° C. incubator for 4 hours. The transfected cells were pelleted by centrifugation and resuspended in 75% Matrigel containing complete culture medium and plated on a 48-well plate. After 7 days, the organoid was passaged and selected against the selection medium. The organoid DNA after extraction was identified by Sanger sequencing to identify the mutation for knock-out organoid.

Western-blot: The nutrition selected organoid was enriched and lysed by RIPA buffer containing protease inhibitor complex V (Roche). The total protein concentration was determined by BSA assay (Pierce) according to manufacture guidance. 50 ug of protein for each line of organoid was loaded into each lane of SDS-PAGE gel. Membrane was stained against rabbit anti-P53 (Novus Biologicals) with 1:500 dilution, rabbit anti-P catenin (abcam) with 1:1000 dilution, and rabbit anti-SMAD4 (abcam) with 1:500 dilution followed with HRP-conjugated secondary anti-rabbit (abcam) antibody with 1:2000 dilution. The signal was normalized with actin by 1:1000 dilution of mouse anti-actin (abcam) followed with 1:2000 HRP-conjugated anti-mouse secondary antibody (abcam). All western-blot images were processed by Bio-rad Image suit.

Orthogonal mucosal injection: Orthotropic transplantation experiment was performed on 12 weeks age B6(Cg)-Rag2^{tm1.1Cgn}/J mice (The Jackson Laboratory). Intestinal organoids were enriched in complete culture medium with 10% Matrigel in a concentration of 4×10⁴ organoids/mL. Orthogonal mucosal injection was performed under optical colonoscopy by using Image 1 H3-Z Spies HD Camera System (part TH100), Image 1 HUB CCU (parts TC200, TC300), 175 Watt D-Light Cold Light Source (part 20133701-1), AIDA HD capture system, and Hopkins Telescope (part 64301AA). For each recipient mouse, 50 uL of organoid solution was delivered to the colon mucosa by optical colonoscopy using a custom injection needle (Hamilton Inc., 33 gauge, small Hub RN NDL, 16 inches long, point 4, 45-degree bevel), a Hamilton syringe, and a Hamilton transfer needle as describe previously (Roper, J., et al. (2017) Nat Biotechnol 35, 569-576) under a colonoscope integrated with working channel (Richard Wolf 1.9 mm/9.5 French pediatric urethroscope) as described previously (Id.).

Histology and Immunohistology: Immunostaining was performed as standard protocol. Briefly, tissue was isolated freshly with a 4 um biopsy punch and immediately fixed with 4% paraformaldehyde followed with standard sectioning paraffin-embedded and H&E staining. For immunohistochemistry, the following primary antibodies were used: β-catenin (abcam) with 1:500 dilution and KRT20 (abcam) with 1:300 dilution. Whole-mount immunostaining was performed according to standard protocol, the organoid was co-stained either with anti-Mucin2 (Novus Biologicals, 1:100 dilution) and anti-chromogranin A (abcam, 1:100 dilution) or with phalliodin (ThermoFisher) and anti-integrin-α6 (Biolegend, 1:50). The nuclei were counterstained with DAPI. The images of organoids were taken by confocal microscopy (Nikon AiR) and bright-field microscopy.

Flow-cytometry: Tissue samples were isolated from each well of the 48-well customized culture plate by a 4 m biopsy puncture and incubated in DMEM medium supplied with 1 mg/mL collagenase type IV (ThermoFisher) and 20 μg/mL DNAseI (ThermoFisher) at 37° C. shaker (450 rpm) for 1 hour. After digestion single cell was obtained through a 70 m cell strainer followed with Cytofix/cytoperm (BD) cell fixation according to standard protocol. Sample was analyzed using a LSR II (BD). Data is analyzed using FlowJo.

Ex vivo tumor organoid mucosal injection and culture: The tissue culture device was manufactured according to previous report. Briefly, a magnet 48-well plate was prepared by laser-cutter. A 10-cm long freshly isolated swine small intestine (as describe above) was washed extensively with cold PBS and dissected longitudinally with surgical scissors. A sterilized 100 m nylon mesh was put in between the bottom plate and tissue in order to create an air-liquid layer. The tissue was put on top of the mesh with lumen side upward and covered with the top plate. Organoid in Matrigel was mechanically disrupted with cold PBS and resuspended in complete culture medium supplemented with 10% Matrigel in a concentration of 4×10⁴ organoid/mL. For each well of the 48-well plate, 50 μL (2000 organoid) of organoid solution was injected into the mucosal layer via 23G syringe needle. The system was maintained in Advance/F12 medium supplemented with 10% FBS and 5% Antibiotic-Antimycotic solution for 3 days for the differentiation of tumor organoids. Medium was changed every 12 hours. At day 4, anti-cancer drug was added to each well transplanted with tumor organoid. 24 hours after drug treatment, the tissue was isolated by biopsy punch followed with flow analysis as described above.

Cell cytotoxicity assay: Drug cytotoxicity against CaCo2, HT29, Colo320DM, and HCT15 cells was performed with CellTiter-Glo Luminescent cell viability assay (Promega) according to manufacture protocol. For engineered colorectal cancer organoids, five days after trypsinization, the matrigel was digested by 1 mg/mL of dispase II (Invitrogen) at 37° C. for 15 minutes. The organoids were further dissociated by physical force and filtrated through a 40 μM nylon strauber (VWR) followed with resuspension in a concentration of 2×10⁴ organoids/mL in complete growth medium containing 2% matrigel. 30 μL of organoid solution was plated on each well of a 384-well Matrigel-precoated plate. After incubation in an incubator for 3 hours, drug with various concentration was added into each well with DMSO final concentration <0.4% in all wells. The ATP level for each well was measured with the same CellTiter-Glo Luminescent cell viability assay to reflect the cytotoxicity of each compound against the CRC organoids. 5-fluorouracil (5-FU), Irinotecan, Oxaliplatin, Doxrubicine, Everolimus, and Lapatinib were purchased from Sigma. Cinobufagin, Regorafenib, Capecitabine and Leucovorin were purchased from MedChem Express.

Machine learning: Data for compounds that interact with P-gp was aggregated from two sources. Firstly, a large database of P-gp substrates curated from DrugBank, MetraBase, and the NIH as previously described (Y. S., et al. Decoding the intestinal transportome through machine learning and tissue engineering. Nat Med (under review)). was relied on. Secondly, data on P-gp inhibitors from the ChEMBL database as previously described (Reker, D., et al. Machine Learning Uncovers Food- and Excipient-Drug Interactions. Cell Rep 30, 3710-3716 e3714 (2020)) was utilized. This lead to a dataset of 1231 known P-gp substrates/inhibitors and 1504 compounds that are known to not interact with P-gp. This dataset was described using RDKit fingerprints (radius 2, 1024 bits) and physicochemical properties (RDKit descriptor calculation) in KNIME (version 3.6). The ability of a random forest model (100 trees based on information gain), a multilayer perceptron (3 hidden layers with 10 neurons per layer), a Naïve Bayes classifier, and Logistic Regression (stochastic average gradient) in stratified ten-fold cross validation was evaluated. The random forest model outperformed the other classifiers and was subsequently employed to predict the P-gp modulation propensity of approved drugs (DrugBank 5) and all FDA-approved inactive ingredients (FDA.gov). These libraries were filtered based on RDKit fingerprint Tanimoto similarity to ensure that predictions were made for compounds that are not part of the training data (T<0.8). Predictions were sorted by maximum confidence (number of trees resulting in positive classification) and the eight candidate adjuvants were chosen manually based on predictive confidence and commercial availability.

Statistical analysis: The screening results from ex vivo platform are representative of four independent experiments. Results from in vitro cell screening were calculated based on replicate experiments. The sigmoidal fitting was carried out using GraphPad Prism to calculate IC₅₀ and a Student's t test was applied to compare the significance for IC₅₀. Differences were considered to be significant at p<0.05. All the results are expressed as mean±standard error (S.D.)

### Example 1: Generation of Colorectal Cancer Organoids Derived from Healthy Intestinal Organoids

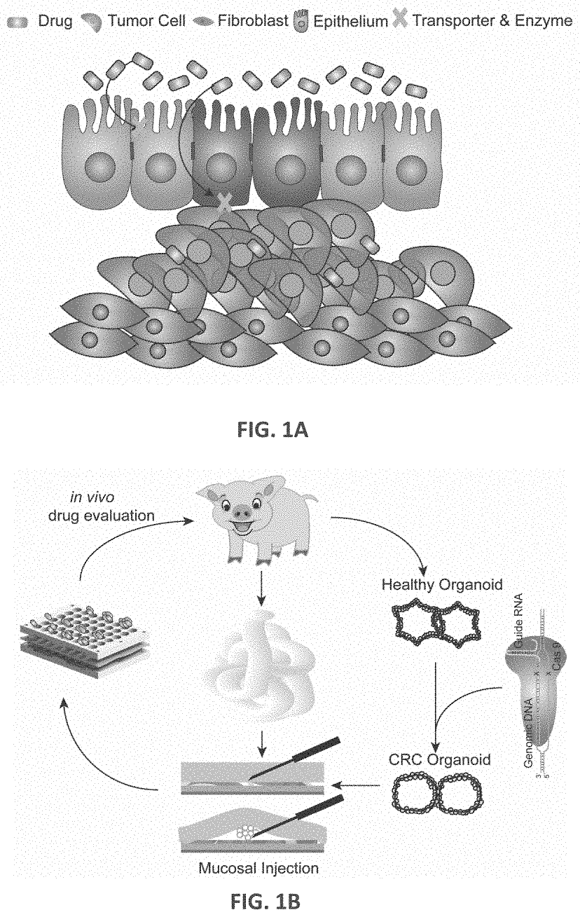

Colorectal tumors commonly evade treatment through changes in drug metabolism and transport. FIG. 1A shows the impact the microenvironment can have on the efficacy of anti-tumor drugs. Specifically, transporters and enzymes located in the epithelium an impact the ability of anti-tumor drugs to reach tumor cells. FIG. 1B shows the workflow chart for generating the colorectal cancer (CRC) platform described herein.

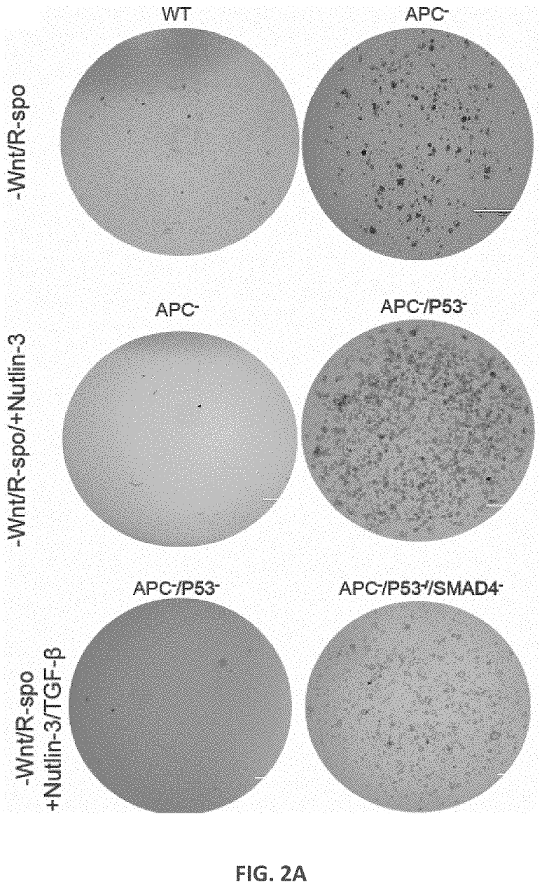

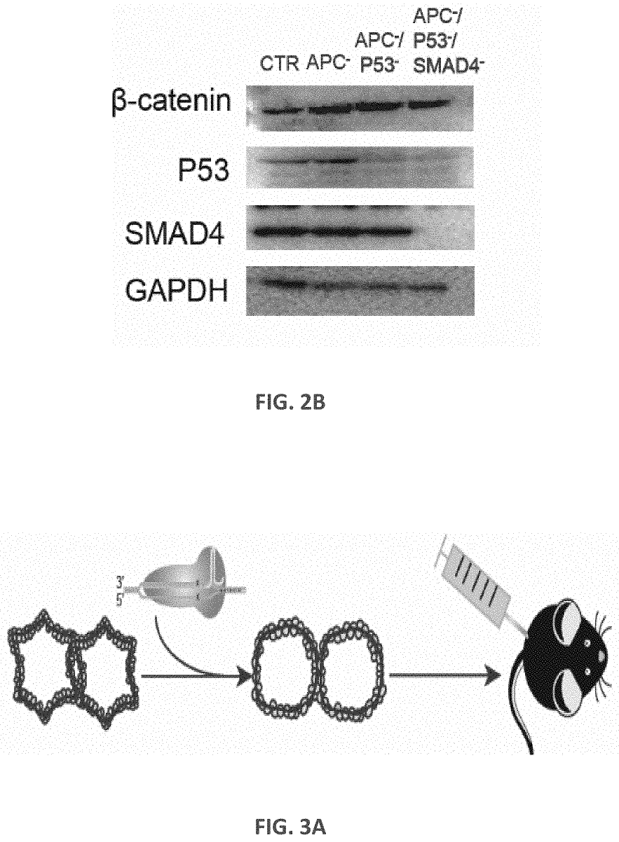

Organoids have been successfully applied in the drug screening process due to their physiological relevance heterogeneity compared to over-simplified cell lines. Therefore, instead of xenografting engineered cancer cell lines, the goal here was to incorporate CRISPR engineered colorectal cancer organoid into healthy swine gut to generate a more physiological relevant cancer screening platform. Driver genes and genetic pathways of colorectal cancer investigations have shown that disruption of key tumor suppressing genes (i.e., APC, TP53, SMAD4, and Kras) could result in development and aggregation of colorectal cancer (Kaz, A. M. & Brentnall, T. A. Genetic testing for colon cancer. Nat Clin Pract Gastroenterol Hepatol 3, 670-679 (2006); Schell, M. J., et al. A multigene mutation classification of 468 colorectal cancers reveals a prognostic role for APC. Nat Commun 7, 11743 (2016); Armaghany, T., Wilson, J. D., Chu, Q. & Mills, G. Genetic alterations in colorectal cancer. Gastrointest Cancer Res 5, 19-27 (2012)). Previous researchers have illustrated that sequential knock-out of the tumor suppression genes APC, TP53, and SMAD4 in healthy human intestinal organoids causes these organoids to become cancerous (i.e., CRC) after orthotropic transplantation in mice (Roper, J., et al. In vivo genome editing and organoid transplantation models of colorectal cancer and metastasis. Nat Biotechnol 35, 569-576 (2017); Matano, M., et al. Modeling colorectal cancer using CRISPR-Cas9-mediated engineering of human intestinal organoids. Nat Med 21, 256-+(2015)). Due to the high genetic and physiological similarity between the human and pig gastrointestinal tract and the ease of collecting these tissues from abattoirs, CRISPR/Cas9 was utilized to sequentially knock-out APC, TP53, and SMAD4 genes on swine intestinal organoid via transfection. The transfected organoids were then selected using media without Wnt3A and R-spondin for APC^{K/O} organoids, media containing nutlin-3 for TP53^{K/O} organoids, and media without murine EGF but supplied with BMP and TGF-β for SMAD^{K/O} organoids as previously reported (Id.). Organoids with successful knock-out of these three genes were able to survive the selection process (FIG. 2A). After expansion, disruption of the targeted loci was also further confirmed by Sanger sequencing (data not shown) and Western blot (FIG. 2B). Immunohistochemistry of the wildtype and knock-out organoids revealed that the CRC organoids exhibited a well-differentiated colonic epithelium feature, such as a complete reversal of apico-basal polarity, while wildtype organoids did not (data not shown).

### Example 2: Development of Colorectal Cancer In Vivo Via Engineered Colorectal Cancer Organoids

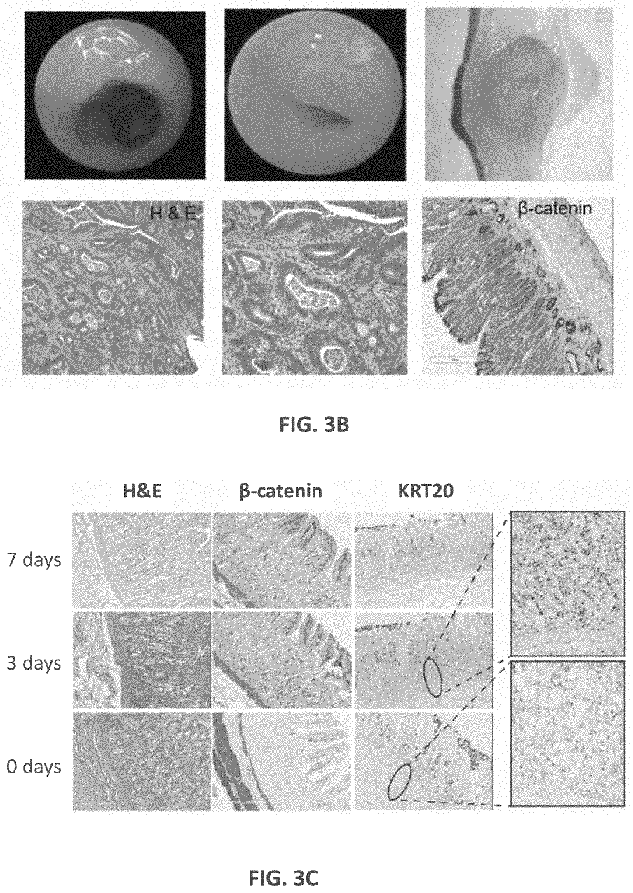

Next, the ability of the swine CRC organoids to develop colorectal cancer in vivo was validated. CRC organoids that have been generated in vitro have previously been shown to engraft and expand into tumors in immune-deficient mice (Roper, J. et al.). Here, an orthotropic mucosal injection of the organoids into Rag2-deficient mice was performed to confirm the tumorigenesis of these organoids in a physiological context. A schematic is shown in FIG. 3A As illustrated in FIG. 3B, 10 days after mucosal injection, the CRC organoids formed tumors that presented similarly to human CRC, indicating that the swine intestinal organoids are clinically relevant and capable of generating colorectal tumors after engraftment.

### Example 3: Engraftment of Colorectal Cancer Organoids in Ex Vivo Swine Tissue

To develop a high-throughput ex vivo CRC drug-screening platform, CRC organoids were integrated into the “intestine-on-a-chip” screening platform previously developed and optimized (von Erlach, T., et al. Robotically handled whole-tissue culture system for the screening of oral drug formulations. Nat Biomed Eng 4, 544-559 (2020)). As investigated extensively according to previous research, it is known that the existence of an air-liquid interface promotes the ex vivo cultural potential of small intestine and colon. Small intestine tissue was immobilized in a 48-well format, and CRC organoids were injected into the mucosal layer of the small intestine within each well. Tumor development was monitored over a 7-day period and the CRC organoid xenograft was excised and analyzed for morphology and protein expression on days 0, 3, and 7 after implantation. CRC organoids exhibited increased expression of carcinoma hall-marker proteins β-catenin, a direct downstream pathway protein of APC, as well as cytokeratin 20 (KRT20), a diagnostic and prognostic marker of CRC²⁶, from day 0 over time (FIG. 3C); notably, KRT20 expression was observed to be more epithelial in staining, which indicates carcinoma and tumor progression as described in previous studies (han, C. W., et al. Gastrointestinal differentiation marker Cytokeratin 20 is regulated by homeobox gene CDX1. Proc Natl Acad Sci USA 106, 1936-1941 (2009)). Compared to tissue analyzed on day 3, day 7 tissue exhibited a large degree of decomposition by H&E staining. Therefore, within the 3-day implantation period the intestine-on-a-chip platform could promote the growth of xenografted CRC organoids in the physiological context of the small intestines.

### Example 4: Development of Colorectal Cancer Platform Using Engineered Organoids and Ex Vivo Swine Tissue

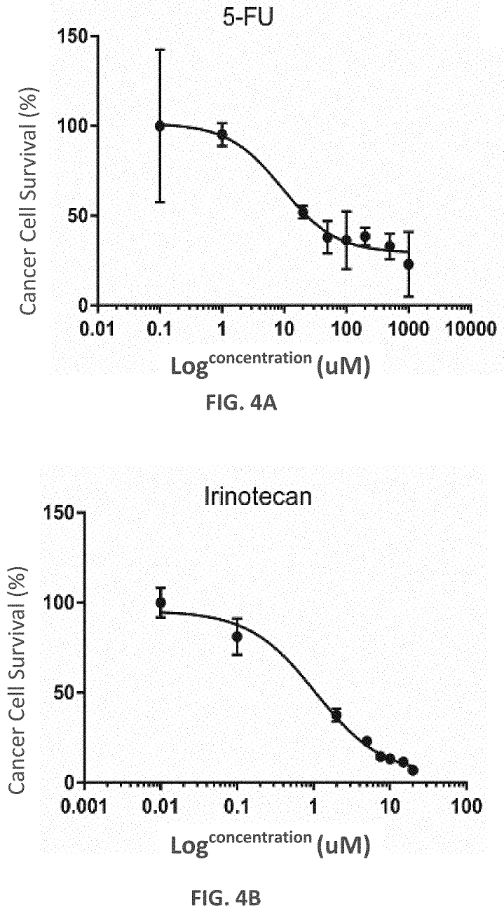

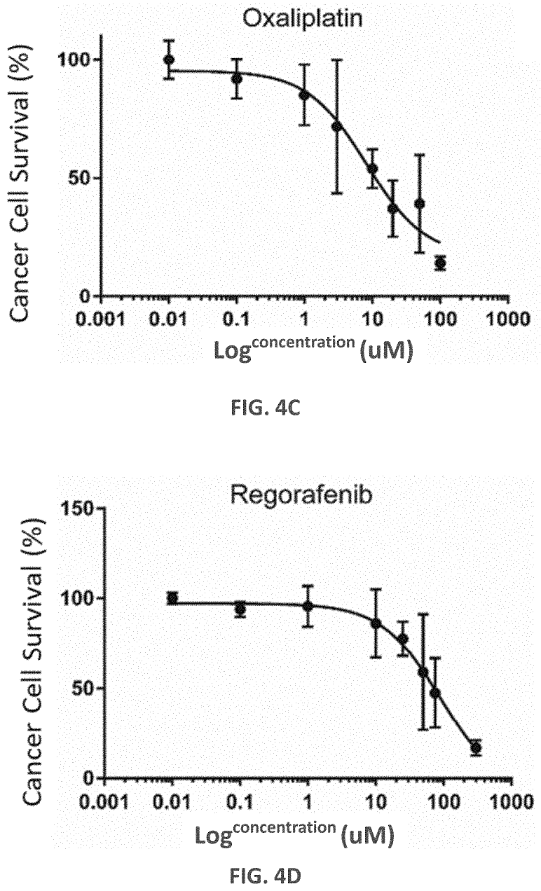

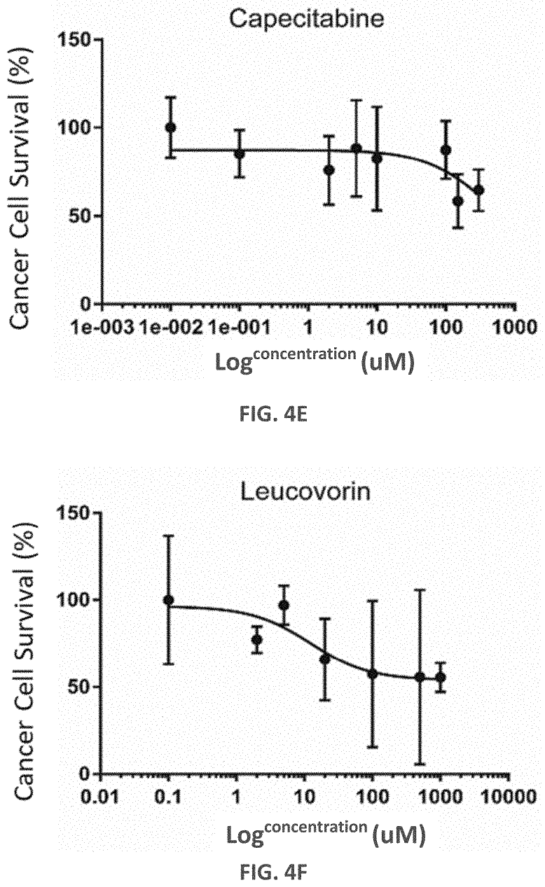

Having shown that the engineered CRC organoids can be engrafted successfully into the intestine-on-a-chip platform, next sensitivity of this platform to anti-cancer drugs and advanced formulations thereof for screening applications was explored. 6 FDA-approved CRC drugs were selected: five drugs that are directly toxic against CRC (5-fluorouracil (5-FU), irinotecan, oxaliplatin, and regorafenib) one pro-drug (capecitabine) whose activation requires a three-step metabolism in both liver and tumor sequentially, and one cytotoxicity modulator drug (leucovorin) which regulates the efficacy of cytotoxicity drugs though its tumor metabolite 5,10-CH₂-THF. To measure the direct drug cytotoxicity to CRCs in the ex vivo platform, CRC organoids were labeled with GFP through lentiviral transduction. CRC organoids were then allowed to grow on the ex vivo intestine platform for 3 days for tumor development. A concentration range for each individual drug was applied to the platform for 24 hours, which allows efficient drug absorption across the tissue. Cytotoxicity was measured by quantifying the decrease in GFP⁺ tumor cells compared with untreated wells via flow cytometry, and the corresponding IC₅₀ value for each drug was calculated through a concentration-dependent symmetrical sigmoidal fitting. As illustrated in FIGS. 4A-4F, all of the four cytotoxic model drugs inhibited tumor cell survival in a dose-dependent effect.

The same drugs were screened in a standard 2D in vitro cell-based assay and noticed that the determined IC₅₀ values differed Table 1. It is hypothesized that such differences could either be attributed to i) drug sensitivity difference between engineered cell line and tumor expansion from tumor organoid; or ii) bioavailability variance due to the absorption barrier and metabolism by tumor microenvironment. The two control drugs capecitabine and leucovoroin did not exhibit significant cytotoxicity, with CRC viability remaining >50% even at high (1 mM) concentrations. They require additional bioconversion or another therapeutic to enable cytotoxicity, so that their sole administration to the CRC platform does not warrant toxicity.

TABLE 1
Tumor cytotoxicity measurements of combinational drugs targeting efflux transporters in the pig ex vivo colorectal cancer platform.

| | | | Doxirubicine + | | Oxiliplatin + | | Lapatinib + |
|---|---|---|---|---|---|---|---|
| System | | Doxirubicine | CBF | Oxiliplatin | CBF | Lapatinib | Everolimus |
| Tissue | IC50 (uM) ± SD | 0.73924 ± 0.082 | 0.3362 ± 0.046 | 8.61 ± 3.375 | 1.14 ± 0.2409 | 0.4993 ± 0.087 | 0.1782 ± 0.0314 |
| | R² | 0.9763 | 0.9589 | 0.8279 | 0.9402 | 0.9457 | 0.9358 |
| | P value | | 0.000141 | | 0.00449 | | 0.00042 |
| Organoid | IC50 (uM) ± SD | 10.07 ± 2.33 | 14.41 ± 4.06 | 17.99 ± 4.72 | 29.28 ± 14.56 | 0.3903 ± 0.21 | 0.4805 ± 0.24 |
| | R² | 0.9676 | 0.9598 | 0.9436 | 0.8465 | 0.8777 | 0.8842 |
| | P value | | 0.6954 | | 0.7901 | | 0.9035 |
| Cell | IC50 (uM) ± SD | 2.854 ± 0.7124 | 2.802 ± 0.4734 | 11.03 ± 1.117 | 12.89 ± 1.799 | NA | NA |
| (Caco2) | R² | 0.9463 | 0.9746 | 0.9905 | 0.9824 | | |
| | P value | | 0.95705 | | 0.47239 | | |
| Cell | IC50 (uM) ± SD | 3.883 ± 2.126 | 6.259 ± 5.906 | NA | NA | NA | NA |
| (HT29) | R² | 0.8598 | 0.6 | | | | |
| | P value | | 0.74144 | | NA | | |
| Cell | IC50 (uM) ± SD | 6.987 ± 1.232 | 9.595 ± 2.004 | 10.49 ± 1.711 | 8.941 ± 1.357 | NA | NA |
| (Colo320DM) | R² | 0.9782 | 0.9737 | 0.9755 | 0.99783 | | |
| | P value | | 0.38305 | | 0.55167 | | |
| Cell | IC50 (uM) | 1.51 ± 0.2256 | 1.84 ± 0.4563 | 15.5 ± 4.988 | 14.72 ± 4.799 | NA | NA |
| (HCT15) | R² | 0.9871 | 0.9651 | 09151 | 0.9124 | | |
| | P value | | 0.58328 | | 0.97002 | | |

One of the major challenges in anti-tumor therapy is the development of drug resistance due to regulation of signaling molecules such as epidermal growth factor receptor (EGFR) and irregulating the expression and activity of drug transporters, which is often omitted in traditional cell screening methods. For example, the combination therapy of irinotecan and cetuximab are largely investigated in clinics and shown promising result in both suppressing progression and reducing liver injury (Paule, B., et al. MDR1 polymorphism role in patients treated with cetuximab and irinotecan in irinotecan refractory colorectal cancer. Med Oncol 27, 1066-1072 (2010)), however, traditional 2D cell culture and organoids would have been unable to detect this useful combination. Next, it was sought to evaluate whether the platform can capture the effect of drug transport on the efficacy of cancer-therapeutics to capture a major mechanism of drug resistance. Previous work has demonstrated that overexpression of drug transporters in tumors contribute to multi-drug resistance due to modification of tumor ECM mediated with drug transporters (Dalton, W. S. The tumor microenvironment: focus on myeloma. Cancer Treat Rev 29 Suppl 1, 11-19 (2003); Jean, C., Gravelle, P., Fournie, J. J. & Laurent, G. Influence of stress on extracellular matrix and integrin biology. Oncogene 30, 2697-2706 (2011)). In CRC, drug efflux pumps have also been investigated for their role in driving tumor immunity and chemoresistance (Robinson, K. & Tiriveedhi, V. Perplexing Role of P-Glycoprotein in Tumor Microenvironment. Front Oncol 10, 265 (2020)).

While multiple distinct proteins compose the efflux system, P-gp is the most predominant and well-characterized drug transporter known to-date and its overexpression is associated with both de novo and acquired resistance to chemotherapy in CRC (Sekine, I., Shimizu, C., Nishio, K., Saijo, N. & Tamura, T. A literature review of molecular markers predictive of clinical response to cytotoxic chemotherapy in patients with breast cancer. Int J Clin Oncol 14, 112-119 (2009)). Cytotoxic drugs to treat various types of tumors are commonly prescribed together with P-gp inhibitors to increase tumor penetration. For example, valspodar has been combined with daunorubicin and etoposide for treating acute myeloid leukemia; tariquidar has been co-administrated with docetaxel and tariquidar for the treatment of solid tumors (Kelly, R. J., et al. A pharmacodynamic study of docetaxel in combination with the P-glycoprotein antagonist tariquidar (XR9576) in patients with lung, ovarian, and cervical cancer. Clin Cancer Res 17, 569-580 (2011)). It was hypothesized that the CRC screening platform could provide an economical pathway to identify suitable combinations of cytotoxic drug and P-gp inhibitors in CRC therapy.

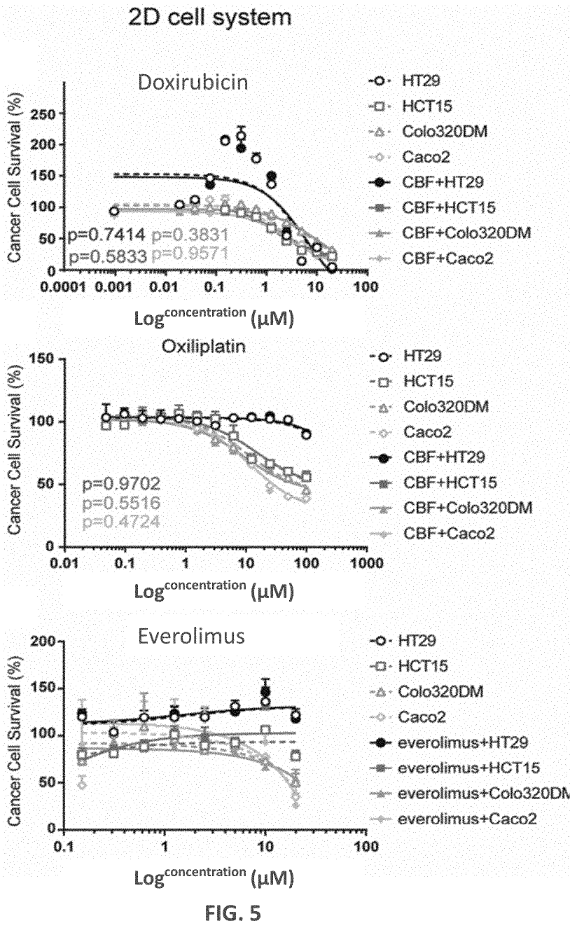

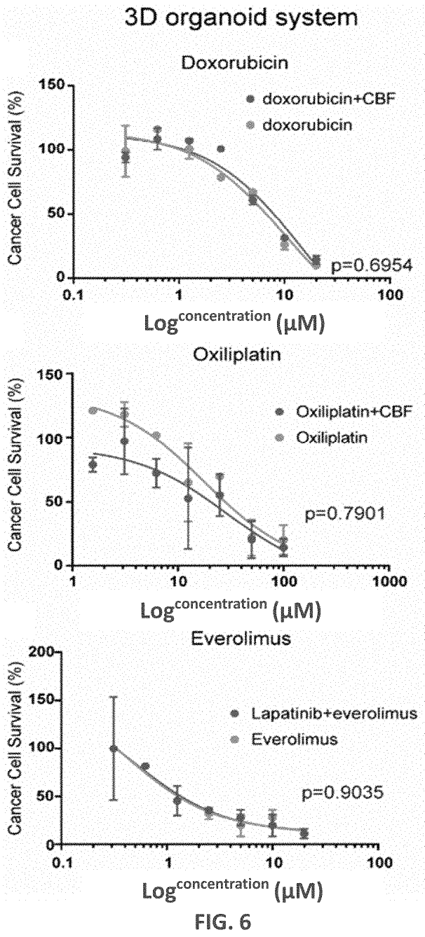

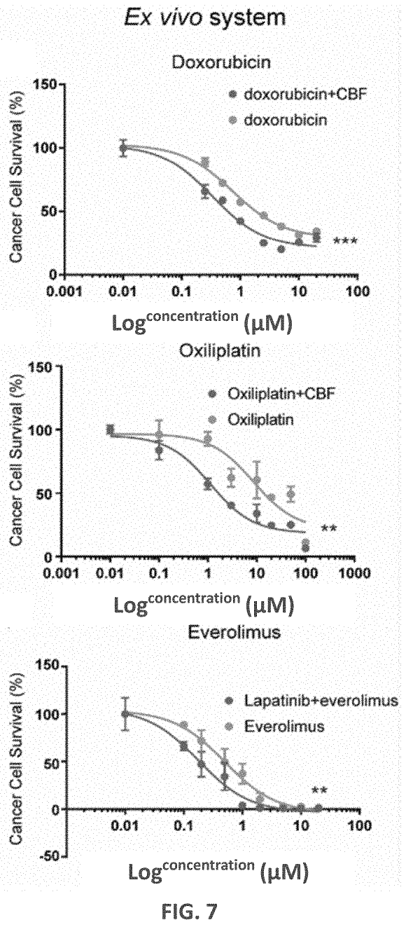

The efficacy of three CRC drugs that are known P-gp substrates (doxorubicin, oxiliplatin, and lapatinib) were evaluated in the presence and absence of two known P-gp inhibitors (cinobufagin (CBF) and everolimus). The ex vivo CRC organoid-in-intestine platform revealed significant improvements in IC₅₀ for doxorubicin, oxaliplatin and everolimous with the P-gp inhibitors (CBF and lapatinib), respectively (p<0.05 (FIG. 7). As a control experiment, the same combinations were evaluated in 4 gastrointestinal cancer cell lines in monoculture (HT29, Caco2, Colo320DM, and HCT15) (FIG. 5) and our CRISPR-engineered CRC organoids (APC⁻/TP53⁻/SMAD4⁻ triple knockout) without transplantation into the native tissue environment (FIG. 6). Neither of these two control models captured a benefit of including P-gp inhibitors to improve tumor targeting. These results showed that due to the fact that the CRC organoid-in-intestine platform leverages the advantages of whole tissue culture that maintains the integrity of various cellular and non-cellular components of the small intestine and spatial arrangement, this platform enables a more holistic method of screening for combination therapies.

### Example 5: Screening of Compounds to Improve Drug Penetration into the Tumor Microenvironment Using Colorectal Cancer Platform

Having established the utility of the CRC organoid-in-intestine screening platform to capture therapeutic benefits of P-gp inhibitor co-administration, the next aim was to prospectively identify novel P-gp inhibitors as functional adjuvants to improve drug penetration into the tumor microenvironment and thereby increase chemotherapeutic efficacy. In previous work, machine learning algorithms were developed to identify P-gp transporter substrates (Y. S., et al. Nat Med (under review)) and inhibitors (Reker, D. Cell Rep (2020)). Here, both datasets were coalesced to train a machine learning model that broadly identifies adjuvants that would decrease P-gp activity through inhibition or substrate competition. In spite of the broad scope of both substrates and inhibitors, a random forest model based on this data exhibits a promising ability to correctly identify modulators from non-modulators, with a retrospective accuracy of 76% in ten-fold cross validations and outcompeting other state-of-the-art machine learning models such as a deep multilayer perceptron, a Naïve Bayes model, or classification based on logistic regression.

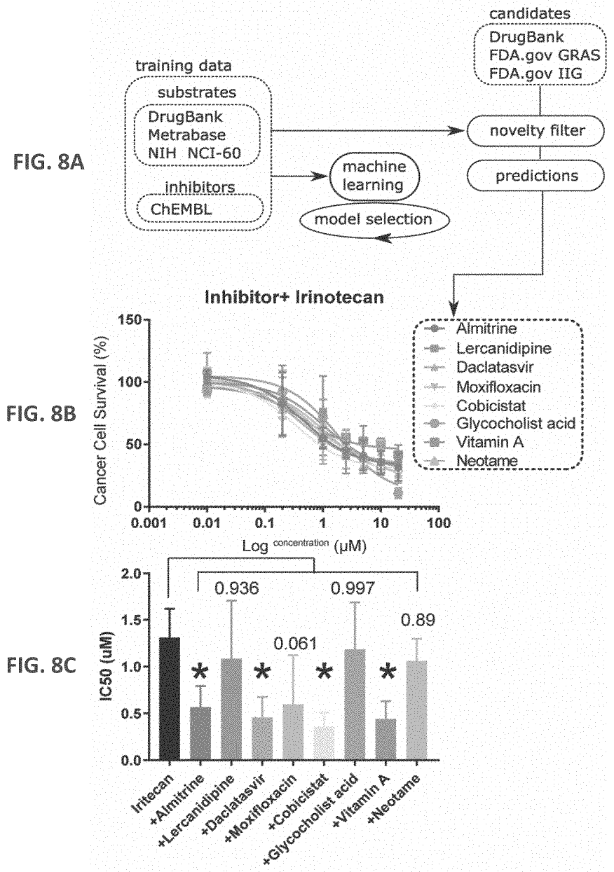

This algorithm was then used to predict the P-gp modulation propensity of FDA-approved drugs, inactive ingredients, and compounds that are Generally Recognized As Safe (GRAS). Individual compounds from the 2656 compound database were filtered to ensure that none of the predicted compounds closely resembled the training database, leading to 2205 structurally distinct compounds. These were then ranked by predictive confidence score. FIG. 8A is a schematic showing the development of this algorithm. Among these top-scored compounds, candidates were selected from each category according to commercial availability and translational interest. These candidates were then combined with irinotecan given its widespread use in clinical practice but strong potential to suffer from developed drug resistance. Four out of the eight predicted P-gp modulators significantly increased the potency irinotecan (FIGS. 8B-8C). Among them, vitamin A is of particular interest because previous work has demonstrated the ability of vitamin A to inhibit P-gp and increase drug uptake (Y. S., et al. Nat Med (under review); Reker, D., et al. Cell Rep (2020)). These findings indicate that the CRC organoid-in-intestine platform has great utility and potential in screening the effect of new combinations of drug in a high-throughput manner.

SEQUENCE LISTING

| | | SEQ |
|---|---|---|
| | | ID |
| Name | Sequence | NO |
| APC fwd-1 | caccTCCACCAGAGCACTACGTTC | 1 |
| APC rev-1 | aaacGAACGTAGTGCTCTGGTGGA | 2 |
| APC fwd-2 | caccGTCTCCTGAACGTAGTGCTC | 3 |
| APC rev-2 | aaacGAGCACTACGTTCAGGAGAC | 4 |
| APC fwd-3 | caccCTAATGTGCTGTGGCACTGT | 5 |
| APC rev-3 | aaacACAGTGCCACAGCACATTAG | 6 |
| APC fwd-4 | caccGGGCTCACTCTGAACGGAGC | 7 |
| APC rev-4 | aaacGCTCCGTTCAGAGTGAGCCC | 8 |
| TP53 fwd-1 | caccTCATCCAGCCAGTTCGTGAC | 9 |
| TP53 rev-1 | aaacGTCACGAACTGGCTGGATGA | 10 |
| TP53 fwd-2 | caccGCAGCTATGATTTCCGTCTA | 11 |
| TP53 rev-2 | aaacTAGACGGAAATCATAGCTGC | 12 |
| TP53 fwd-3 | caccGTCCCCAGTCACGAACTGGC | 13 |
| TP53 rev-3 | aaacGCCAGTTCGTGACTGGGGAC | 14 |
| TP53 fwd-4 | caccTGCTGTCCCCAGTCACGAAC | 15 |
| TP53 rev-4 | aaacGTTCGTGACTGGGGACAGCA | 16 |
| SMAD fwd-1 | caccTGTCCTATTGTTACTGTCGA | 17 |
| SMAD rev-1 | aaacTCGACAGTAACAATAGGACA | 18 |
| SMAD fwd-2 | caccTCAAGTAGGAGAGACGTTTA | 19 |
| SMAD rev-2 | aaacTAAACGTCTCTCCTACTTGA | 20 |
| SMAD fwd-3 | caccGCAATGGAACACCAATACTC | 21 |
| SMAD rev-3 | aaacGAGTATTGGTGTTCCATTGC | 22 |
| SMAD fwd-4 | caccTATCCATCGACAGTAACAAT | 23 |
| SMAD rev-4 | aaacATTGTTACTGTCGATGGATA | 24 |

1. An ex vivo tissue composition comprising (i) a tissue explant from a mammalian source tissue in planar contact with a substrate; and (ii) a population of cells or tissue within the tissue explant, wherein the population of cells or tissue provides one or more biological functions of normal or diseased cells or tissue, or one or more markers of said biological functions.

2. The tissue composition of claim 1, wherein the source tissue is selected from gastrointestinal tissue, liver tissue, heart tissue, skin tissue, pancreas tissue, and kidney tissue.

3. An ex vivo tissue composition comprising (i) a tissue explant in planar contact with a substrate thereby providing a luminal and a basolateral surface, wherein the tissue explant comprises epithelium from a mammalian gastrointestinal tract comprising an architecture, wherein said tissue explant comprises said architecture; and (ii) a population of cells or tissue within the tissue explant.

4. The tissue composition of claim 1, wherein the population of cells or tissue is a xenograft, an allograft, an autograft, a biopsy from a subject, and/or derived from a primary tissue.

5.-7. (canceled)

8. The tissue composition of claim 4, wherein the subject is a human.

9. The tissue composition of claim 1, wherein the population of cells or tissue is an organoid.

10. The tissue composition of claim 9, wherein the organoid comprises cells of an immortalized cell line, primary cells, stem cells, cells from a normal tissue or cells from a diseased tissue.

11.-14. (canceled)

15. The tissue composition of claim 10, wherein diseased tissue is a cancerous tissue or a tissue comprising a population of cells comprising at least one genetic mutation.

16.-19. (canceled)

20. The tissue composition of claim 15, wherein the genetic mutation is in at least one of the APC, p53, or SMAD4 genes.

21.-25. (canceled)

26. The tissue composition of claim 9, wherein the organoids form tumors after placement within the tissue explant.

27. The tissue composition of claim 1, wherein the substrate comprises a plurality of microwells, and wherein the population of cells or tissue is placed within the tissue explant in a location that corresponds to a microwell.

28. The tissue composition of claim 1, wherein the population of cells or tissue is derived from tissue of the gastrointestinal tract, liver, pancreas, kidney, spleen, lung, skin or heart.

29. (canceled)

30. The tissue composition of claim 3, wherein the architecture comprises epithelial cells having a polarity, small intestine epithelium, circular muscular layer, mucosa layer, submucosa layer, and/or intestinal villi.

31. The tissue composition of claim 1, wherein the tissue explant comprises a fully intact extracellular matrix, wherein the fully intact extracellular matrix comprises lamina propria, lamina muscularis, or lamina propria and lamina muscularis.

32.-35. (canceled)

36. The tissue composition of claim 3, wherein the tissue explant comprises an intestinal mucosal layer and a submucosal layer.

37. The tissue composition of claim 36, wherein the population of cells or tissue is between the mucosa layer and the submucosa layer.

38. The tissue composition of claim 1, wherein the composition is maintained in culture for 24 hours, 2 days, 3 days, 1 week, 2 weeks, 3 weeks, 4 weeks, or more.

39. The tissue composition of claim 1, wherein the composition does not require an exogenous growth factor to be maintained in culture.

40.-41. (canceled)

42. The tissue composition of claim 1, wherein the population of cells or tissue is injected into the tissue explant.

43.-56. (canceled)

57. An ex vivo tissue composition comprising: (i) a tissue explant in planar contact with a substrate thereby providing a luminal and a basolateral surface, wherein the tissue explant comprises intestinal epithelium from a source tissue, wherein said source tissue comprises intestinal epithelium from a large mammalian gastrointestinal tract, wherein said source tissue comprises an architecture comprising epithelial cells having a polarity, wherein the tissue explant comprises said architecture; and (ii) a tumorigenic intestinal organoid within the tissue explant.

58.-91. (canceled)

92. The tissue composition of claim 1, wherein the substrate comprises a first plate comprising the plurality of microwells and a second plate, wherein the tissue explant is between the first and second plates.

93.-97. (canceled)

98. A method for determining the cytotoxic effect of a candidate drug on cancer cells, comprising:
(i) providing the tissue composition of claim 1;
(ii) contacting the tissue composition with the candidate drug; and
(iii) measuring cancer cell death within the population of cells, tissue or tumorigenic organoid after treatment with the candidate drug for a period of time, thereby determining the cytotoxic effect of the candidate drug on cancer cells.

99. (canceled)

100. A system for use in a high-throughput colorectal cancer cytotoxicity screening assay, wherein the system comprises:
(i) a substrate comprising a plurality of microwells;
(ii) a tissue explant from a source tissue comprising epithelium from a large mammalian gastrointestinal tract, wherein the gastrointestinal tract epithelium comprises epithelial cells having a polarity in the tissue explant; and
(iii) a tumorigenic colon organoid injected within the tissue explant;
thereby allowing measurement of cytotoxicity toward the tumorigenic organoid through the tissue explant.
